(19) United States
(12) Patent Application Publication
(10) Pub. No.: US 2026/0061053 A1
(43) Pub. Date: Mar. 5, 2026

(54) CD19-DIRECTED CHIMERIC ANTIGEN RECEPTOR CELL THERAPY FOR TREATING AUTOIMMUNE AND NEUROLOGICAL DISEASES

(71) Applicant: Juno Therapeutics, Inc., Seattle, WA (US)

(72) Inventors: Ashley KOEGEL, San Francisco, CA (US); Timothy CAMPBELL, San Francisco, CA (US); Tyler Jordan KOOP, Pennington, NJ (US); Renee Ann Sehee RANDAZZO, Millstone Township, NJ (US); Alexis MELTON, Belmont, CA (US); Burhan Zafar CHAUDHRY, Wayne, PA (US); Nivedita GULATI, Media, PA (US)

(73) Assignee: Juno Therapeutics, Inc., Seattle, WA (US)

(21) Appl. No.: 19/312,063

(22) Filed: Aug. 27, 2025

Related U.S. Application Data
(60) Provisional application No. 63/688,226, filed on Aug. 28, 2024.

(51) Int. Cl. A61K 40/11; A61K 40/31; A61K 40/41; A61P 37/06; C07K 14/725; C07K 16/28

(52) U.S. Cl. CPC A61K 40/11; A61K 40/31; A61K 40/416; A61P 37/06; C07K 14/7051; C07K 16/2803

(57) ABSTRACT

Provided herein are adoptive cell therapy methods and uses involving the administration of a dose of T cells expressing a CD19-directed chimeric antigen receptor for treating subjects with autoimmune and neurological disease and disorders and related methods, compositions, uses and articles of manufacture.

### CROSS-REFERENCE TO RELATED APPLICATIONS

This application claims priority from U.S. provisional application No. 63/688,226 filed Aug. 28, 2024, entitled “CD19-DIRECTED CHIMERIC ANTIGEN RECEPTOR CELL THERAPY FOR TREATING AUTOIMMUNE AND NEUROLOGICAL DISEASES”, the contents of which are incorporated by reference in its entirety.

### INCORPORATION BY REFERENCE OF SEQUENCE LISTING

The present application is being filed with a Sequence Listing in electronic format. The Sequence Listing is provided as a file entitled 735042029800SeqList.xml, created on Aug. 22, 2025, which is 132,871 bytes in size. The information in the electronic format of the Sequence Listing is incorporated by reference in its entirety.

### FIELD

The present disclosure relates in some aspects to adoptive cell therapy involving the administration of a dose of T cells expressing a CD19-directed chimeric antigen receptor for treating subjects with autoimmune and neurological diseases and disorders and related methods, compositions, uses and articles of manufacture.

### BACKGROUND

Autoimmune disease relates to a wide range of diseases and disorders, including neurological disorders, characterized by dysregulation of the immune system, including in many cases B cell involvement. Many patients eventually relapse or become refractory to available therapies, and second-line, third-line, and particularly fourth-line treatments are limited. In some cases, no approved therapies are available. Effective therapies for patients with systemic autoimmune diseases or neurological disorders, particularly those who have failed one or more prior therapy, are needed. Provided are methods and uses that meet such needs.

### SUMMARY

Provided herein is a method of treating a subject having Myasthenia gravis (MG), the method comprising administering a dose of CD19-directed genetically modified T cells to a subject having or suspected of having Myasthenia gravis (MG), wherein the T cells of the dose are positive for expression of a chimeric antigen receptor (CAR) that binds CD19 and the dose is from 1×10⁶ to 50×10⁶ CAR-positive, optionally viable, T cells, wherein the subject has severe disease that is refractory to three or more prior therapies for treating MG.

Also provided herein is a method for reducing Myasthenia gravis (MG) disease activity, the method comprising administering a dose of CD19-directed genetically modified T cells to a subject having or suspected of having Myasthenia gravis (MG), wherein the T cells of the dose are positive for expression of a chimeric antigen receptor (CAR) that binds CD19 and the dose is from 1×10⁶ to 50×10⁶ CAR-positive, optionally viable, T cells, wherein the subject has severe disease that is refractory to three or more prior therapies for treating MG.

In some embodiments, the prior therapy is eculizumab, efgartigimod, or Rystiggo. In some embodiments, the MG is anti-AChR antibody positive. In some embodiments, the MG is anti-MuSK antibody positive. In some embodiments, the MG is anti-LRP4 positive.

Also provided herein is a method of treating a subject having primary Sjogren's Disease (SjD), the method comprising administering a dose of CD19-directed genetically modified T cells to a subject having or suspected of having primary Sjogren's Disease (SjD), wherein the T cells of the dose are positive for expression of a chimeric antigen receptor (CAR) that binds CD19 and the dose is from 1×10⁶ to 50×10⁶ CAR-positive, optionally viable, T cells, wherein the subject is characterized with refractory disease that is refractory to at least one prior therapy for treating SjD and/or with extraglandular disease.

Also provided herein is a method for reducing primary Sjogren's Disease (SjD) disease activity, the method comprising administering a dose of CD19-directed genetically modified T cells to a subject having or suspected of having primary Sjogren's Disease (SjD), wherein the T cells of the dose are positive for expression of a chimeric antigen receptor (CAR) that binds CD19 and the dose is from 1×10⁶ to 50×10⁶ CAR-positive, optionally viable, T cells, wherein the subject is characterized with refractory disease that is refractory to at least one prior therapy for treating SjD and/or with extraglandular disease.

In some embodiments, the subject has refractory disease to at least one prior therapy. In certain embodiments, the disease is a glandular disease. In certain embodiments, the disease is an extraglandular disease. In some embodiments, at least one prior therapy is a hydroxychloroquine, oral glucocorticoid, immunosuppressive agent or an anti-CD20 antibody, optionally wherein the anti-CD20 antibody is Rituximab. In some embodiments, the subject has extraglandular disease. In certain embodiments, the subject has severe extraglandular disease is characterized by one or more of cutaneous vasculitis, renal involvement, peripheral nervous system (PNS) involvement, interstitial lung disease (ILD), or cryoglobulinemia. In some embodiments, the subject has anti-Sjogren's syndrome A (SSA) antibodies or anti-Sjogren's syndrome B (SSB) antibodies. In some embodiments, the subject has high disease activity as determined by the European League Against Rheumatism (EULAR) Sjagren Syndrome Disease Activity Index (ESSDAI) score of 14 or higher, optionally characterized by multiple organ involvement.

Also provided herein is a method of treating a subject having ANCA-associated vasculitis (AAV), the method comprising administering a dose of CD19-directed genetically modified T cells to a subject having or suspected of having ANCA-associated vasculitis (AAV), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing ANCA-associated vasculitis (AAV), the method comprising administering a dose of CD19-directed genetically modified T cells to a subject having or suspected of having ANCA-associated vasculitis (AAV), wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the subject has a Birmingham Vasculitis Activity Score (BVAS) greater than 16. In some embodiments, the subject has relapsed following remission after treatment with a prior therapy, optionally wherein the relapse is within 5 years of receiving the prior therapy. In certain embodiments, the prior therapy is a corticosteroid, optionally a high-dose glucocorticoid, an anti-CD20 antibody, optionally rituximab, a complement inhibitor or an intravenous immunoglobulin. In some embodiments, the subject is treatment refractory, optionally wherein the subject fails treatment with an immunosuppressant or disease-modifying antirheumatic drug (DMARDs) and intravenous immunoglobulin. In some embodiments, the subject is cytoplasmic-ANCA or proteinase-3-ANCA positive. In some embodiments, the subject is characterized by severe disease with organ involvement or organ damage, optionally wherein the organ is renal or pulmonary.

Also provided herein is a method for post-induction maintenance therapy for treating ANCA-associated vasculitis (AAV), the method comprising administering a dose of CD19-directed genetically modified T cells to a subject having received an induction therapy for treating ANCA-associated vasculitis (AAV), wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the induction therapy comprises one or more of cyclophosphamide, an anti-CD20 antibody (e.g., rituximab), avacophan, methotrexate or mycophenolate mofetil. In some embodiments, the induction therapy comprises an anti-CD20 antibody, optionally rituximab, in combination with a corticosteroid, optionally a glucocorticoid.

Also provided herein is a method of treating a subject having Rheumatoid Arthritis (RA), the method comprising administering a dose of CD19-directed genetically modified T cells to a subject having or suspected of having Rheumatoid Arthritis (RA), wherein the T cells of the dose are positive for expression of a chimeric antigen receptor (CAR) that binds CD19 and the dose is from 1×10⁶ to 50×10⁶ CAR-positive, optionally viable, T cells, wherein the subject has severe disease that is refractory to three or more prior therapies for treating RA.

Also provided herein is a method for reducing Rheumatoid Arthritis (RA) disease activity, the method comprising administering a dose of CD19-directed genetically modified T cells to a subject having or suspected of having Rheumatoid Arthritis (RA), wherein the T cells of the dose are positive for expression of a chimeric antigen receptor (CAR) that binds CD19 and the dose is from 1×10⁶ to 50×10⁶ CAR-positive, optionally viable, T cells, wherein the subject has severe disease that is refractory to three or more prior therapies for treating RA.

In some embodiments, the at least two of the three or more prior therapies are at least two previous treatments with a disease-modifying anti-rheumatic drug (DMARD). In some embodiments, wherein the DMARD is methotrexate, sulfasalazine, hydroxychloroquine, and leflunomide; a TNF antagonist (e.g., adalimumab, etanercept or infliximab); or a targeted synthetic DMARD (e.g., JAK inhibitor, such as baricitinib and tofacitinib). In some embodiments, the subject has severe extra-articular disease, optionally characterized by one or more of renal involvement, peripheral nervous system (PNS) involvement, interstitial lung disease (ILD). In some embodiments, the subject has a high DAS28 score, optionally a score of greater than 5.1. In some embodiments, the subject is positive for rheumatoid factor (RF) and anti-cyclic citrullinated peptide antibody (ACPA). In some embodiments, the subject has disease characterized by severe organ involvement that includes renal involvement, peripheral nervous system (PNS) involvement, or interstitial lung disease (ILD).

Also provided herein is a method of treating a subject having Autoimmune Encephalitis (AE), the method comprising administering a dose of CD19-directed genetically modified T cells to a subject having or suspected of having Autoimmune Encephalitis (AE), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Autoimmune Encephalitis (AE) disease activity, the method comprising administering a dose of CD19-directed genetically modified T cells to a subject having or suspected of having Autoimmune Encephalitis (AE), wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the subject has severe disease that is refractory to one or more prior therapies. In certain embodiments, the one or more prior therapy is two or more prior therapies. In some embodiments, the one or more prior therapy is one or more of a high-dose steroid, intravenous immunoglobulins (IVIG), intravenous methylprednisolone (IVMP), plasma exchange (PLEX) or immunosuppressant. In some embodiments, the subject has relapsed or is refractory to two prior lines of therapy, wherein the first line of therapy is a high-dose steroid, intravenous immunoglobulins (IVIG), intravenous methylprednisolone (IVMP), or a plasma exchange (PLEX) and the second line of therapy is an immunosuppressant. In some embodiments, the immunosuppressant is an anti-CD20 antibody, optionally Rituximab; rituximab and tocilizumab; cyclophosphamide; mycophenolate mofetil; or azathioprine.

Also provided herein is a method for post-induction maintenance therapy for treating Autoimmune Encephalitis (AE), the method comprising administering a dose of CD19-directed genetically modified T cells to a subject having received an induction therapy for treating Autoimmune Encephalitis (AE), wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the induction therapy comprises an anti-CD20 antibody, optionally rituximab. In some embodiments, the induction therapy comprises an anti-CD20 antibody, optionally rituximab, in combination with a corticosteroid, optionally a glucocorticoid. In some embodiments, the subject has anti-NMDAR antibodies or anti-LGI1 antibodies.

Also provided herein is a method of treating a subject having Pemphigus, the method comprising administering a dose of CD19-directed genetically modified T cells to a subject having or suspected of having Pemphigus, wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Pemphigus disease activity, the method comprising administering a dose of CD19-directed genetically modified T cells to a subject having or suspected of having Pemphigus, wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the subject has disease that has relapsed or is refractory to one or more prior therapies for treating Pemphigus. In certain embodiments, the one or more prior therapy is a corticosteroid, azathioprine, methotrexate, an anti-CD20 antibody, optionally rituximab, or efgartigimod.

Also provided herein is a method of treating a subject having Membranous Nephropathy (MN), the method comprising administering a dose of CD19-directed genetically modified T cells to a subject having or suspected of having Membranous Nephropathy (MN), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Membranous Nephropathy (MN) disease activity, the method comprising administering a dose of CD19-directed genetically modified T cells to a subject having or suspected of having Membranous Nephropathy (MN), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Immunoglobulin G4-related disease (IgG4-RD), the method comprising administering a dose of CD19-directed genetically modified T cells to a subject having or suspected of having Immunoglobulin G4-related disease (IgG4-RD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Immunoglobulin G4-related disease (IgG4-RD) disease activity, the method comprising administering a dose of CD19-directed genetically modified T cells to a subject having or suspected of having Immunoglobulin G4-related disease (IgG4-RD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Neuromyelitis optica spectrum disorder (NMOSD), the method comprising administering a dose of CD19-directed genetically modified T cells to a subject having or suspected of having Neuromyelitis optica spectrum disorder (NMOSD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Neuromyelitis optica spectrum disorder (NMOSD) disease activity, the method comprising administering a dose of CD19-directed genetically modified T cells to a subject having or suspected of having Neuromyelitis optica spectrum disorder (NMOSD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Stiff-person syndrome (SPS), the method comprising administering a dose of CD19-directed genetically modified T cells to a subject having or suspected of having Stiff-person syndrome (SPS), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Stiff-person syndrome (SPS) disease activity, the method comprising administering a dose of CD19-directed genetically modified T cells to a subject having or suspected of having Stiff-person syndrome (SPS), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Irritable bowel disease (IBD), the method comprising administering a dose of CD19-directed genetically modified T cells to a subject having or suspected of having Irritable bowel disease (IBD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Irritable bowel disease (IBD) disease activity, the method comprising administering a dose of CD19-directed genetically modified T cells to a subject having or suspected of having Irritable bowel disease (IBD), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Thrombotic Thrombocytopenia Purpura (TTP), the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Thrombotic Thrombocytopenia Purpura (TTP) disease activity, the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Autoimmune hemolytic anemia (AIHA), the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Autoimmune hemolytic anemia (AIHA) disease activity, the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having Immune thrombocytopenia (ITP), the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing Immune thrombocytopenia (ITP) disease activity, the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×10⁶ to 50×10⁶ CAR-positive, optionally viable, T cells. In some embodiments, the ITP is chronic ITP.

Also provided herein is a method of treating a subject having chronic Immune thrombocytopenia (cITP), the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing chronic Immune thrombocytopenia (cITP) disease activity, the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having IgA nephropathy, the method comprising administering a dose of CD19-directed genetically modified T cells to a subject having or suspected of having IgA nephropathy, wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing IgA nephropathy disease activity, the method comprising administering a dose of CD19-directed genetically modified T cells to a subject having or suspected of having IgA nephropathy, wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having bullous pemphigoid (BP), the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is method for reducing bullous pemphigoid (BP) disease activity, the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method of treating a subject having ulcerative colitis (UC), the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×10⁶ to 50×10⁶ CAR-positive, optionally viable, T cells.

Also provided herein is a method for reducing ulcerative colitis (UC) disease activity, the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×10⁶ to 50×10⁶ CAR-positive, optionally viable, T cells.

In some embodiments, the systemic autoimmune disease is a relapsed or refractory disease to one or more prior therapies for treating the disease.

In some embodiments, the one or more prior therapies are two or more prior therapies for treating the disease.

In some embodiments, at least one of the one or more prior therapies is an anti-CD20 antibody, optionally rituximab.

In some embodiments, the systemic autoimmune disease is a severe disease.

In some embodiments, the dose is at or about 1×10⁶ to 40×10⁶ CAR-positive, optionally viable, T cells.

In some embodiments, the dose is at or about 1×10⁶ to 25×10⁶ CAR-positive, optionally viable, T cells.

In some embodiments, the dose is at or about 5×10⁶ CAR-positive, optionally viable, T cells.

In some embodiments, the dose is at or about 10×10⁶ CAR-positive, optionally viable, T cells.

In some embodiments, the dose is at or about 25×10⁶ CAR-positive, optionally viable, T cells.

In some embodiments, the dose is at or about 50×10⁶ CAR-positive, optionally viable, T cells.

In some embodiments, the T cells are autologous to the subject.

In some embodiments, the method further comprises obtaining a leukapheresis sample from the subject for manufacturing the composition comprising engineered T cells.

In some embodiments, prior to the administration, the subject has been preconditioned with a lymphodepleting therapy.

In some embodiments, the method further comprises, immediately prior to the administration of the dose of CD19-directed genetically modified T cells, administering a lymphodepleting therapy to the subject, wherein the lymphodepleting therapy comprises the administration of fludarabine and/or cyclophosphamide.

In some embodiments, the administration of the dose of CD19-directed genetically modified T cells and/or the lymphodepleting therapy is carried out via outpatient delivery.

In some embodiments, the lymphodepleting therapy comprises the administration of fludarabine at 30 mg/m² body surface area of the subject, daily, and cyclophosphamide at 300 mg/m² body surface area of the subject, daily, each for 3 days.

In some embodiments, the dose of CD19-directed genetically modified T cells is administered between at or about 48 hours and at or about 9 days, inclusive, after completion of the lymphodepleting therapy.

In some embodiments, the dose of CD19-directed genetically modified T cells is administered to the subject by intravenous infusion.

In some embodiments, the CAR comprises an extracellular antigen-binding domain that binds CD19, a transmembrane domain, and an intracellular signaling domain. In some embodiments, the CAR comprises a hinge spacer between the extracellular antigen-binding domain and the transmembrane domain, optionally wherein the hinge spacer is an immunoglobulin hinge or a CD8a hinge. In some embodiments, the extracellular antigen-binding domain is an FMC63 monoclonal antibody-derived single chain variable fragment (scFv). In some embodiments, the extracellular antigen-binding domain comprises a variable heavy chain set forth in SEQ ID NO:10 and a variable light chain set forth in SEQ ID NO:11. In some embodiments, the scFv is set forth as SEQ ID NO: 14. In some embodiments, the extracellular antigen-binding domain is a Hu19 single chain variable fragment (scFv). In some embodiments, the extracellular antigen-binding domain comprises a variable heavy chain set forth in SEQ ID NO:50 and a variable light chain set forth in SEQ ID NO:48. In some embodiments, the extracellular antigen-binding domain comprises in order a variable light chain set forth in SEQ ID NO: 48, a linker peptide set forth in SEQ ID NO: 49, and a variable heavy chain set forth in SEQ ID NO: 50.

In some embodiments, the CAR is a monospecific CAR directed to CD19.

In some embodiments, the CAR is a tandem bispecific CAR directed against CD19 and at least one other antigen expressed on B cells. In some embodiments, the other antigen expressed on B cells is selected from the group consisting of CD20, CD19, CD22, ROR1, BCMA, CD45, CD21, CD5, CD33, Igkappa, Iglambda, CD79a, CD79b or CD30. In some embodiments, the other antigen expressed on B cells is CD20. In some embodiments, the extracellular antigen-binding domain comprises a variable heavy chain and a variable light chain derived from a CD20 antibody selected from the group consisting of Leu16, C2B8, 11B8, 8G6-5, 2.1.2 and GA101. In some embodiments, the transmembrane domain is a CD28 transmembrane domain. In some embodiments, the transmembrane domain is a transmembrane domain from CD28, optionally a transmembrane domain that comprises the sequence of amino acids set forth in SEQ ID NO: 34 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:34. In some embodiments, the intracellular signaling domain comprises a 4-1BB costimulatory domain and a CD3zeta activation domain.

In some embodiments, the CAR comprises, in order from N- to C-terminus, an FMC63 monoclonal antibody-derived single chain variable fragment (scFv), IgG4 hinge region, a CD28 transmembrane domain, a 4-1BB (CD137) costimulatory domain, and a CD3 zeta signaling domain. In some embodiments, the 4-1BB costimulatory domain is or comprises the sequence set forth in SEQ ID NO: 42 or a variant thereof having at least 85%, 86%, 87%, 88%, 89%, 90%, 91%, 92%, 93%, 94%, 95%, 96%, 97%, 98%, 99% or more sequence identity to SEQ ID NO:42. In some embodiments, the CD3zeta signaling domain is or comprises the sequence set forth in SEQ ID NO: 37, 38 or 39 or a sequence having at least 85%, 86%, 87%, 88%, 89%, 90%, 91%, 92%, 93%, 94%, 95%, 96%, 97%, 98%, 99% or more sequence identity thereto.

In some embodiments, the CAR contains in order from N-terminus to C-terminus: an extracellular antigen-binding domain that is the scFv set forth in SEQ ID NO: 14, the spacer set forth in SEQ ID NO:29, the transmembrane domain set forth in SEQ ID NO:34, the 4-1BB costimulatory signaling domain set forth in SEQ ID NO:42, and the signaling domain of a CD3-zeta (CD3( ) chain set forth in SEQ ID NO:37.

In some embodiments, the CAR comprises the amino acid sequence set forth in SEQ ID NO:43 or a sequence having at least 85%, 86%, 87%, 88%, 89%, 90%, 91%, 92%, 93%, 94%, 95%, 96%, 97%, 98%, 99% or more sequence identity to SEQ ID NO: 43.

In some embodiments, the dose of T cells comprises CD4⁺ T cells expressing the CAR and CD8⁺ T cells expressing the CAR.

In some embodiments, the dose of T cells comprises CD4⁺ T cells expressing the CAR and CD8⁺ T cells expressing the CAR at a ratio between about 1:5 and about 5:1, optionally at a ratio between about 1:3 and about 3:1.

In some embodiments, at least or at least about 90% of the cells in the composition are CD3+ cells.

In some embodiments, at least or at least about 91%, at least or at least about 92%, at least or at least about 93%, at least or at least about 94%, at least or at least about 95%, or at least or at least about 96% of the cells in the composition are CD3⁺ cells.

In some embodiments, at least 25% of the T cells in the composition are CAR+ T cells.

In some embodiments, at least 30%, at least 35%, at least 40%, at least 45% or at least 50% of the T cells in the composition are CAR+ T cells.

In some embodiments, at least 80% of the T cells in the composition are, optionally viable, T cells, optionally wherein viability is determined by staining for acridine orange (AO) and propidium iodide (PI).

In some embodiments, the subject does not receive administration of immunosuppressant for treating the disease after administering the dose of CD19-directed genetically modified T cells.

In some embodiments, the subject is human.

### BRIEF DESCRIPTION OF THE DRAWINGS

FIGS. 1A-1F depict the CAR transgene levels (FIG. 1A), serum IgG (FIG. 1B), serum IgA (FIG. 1C), the number of neutrophils (FIG. 1D), the number of total lymphocytes (FIG. 1E), and the number of platelets (FIG. 1F) in human patients after treatment with 10×10⁶ or 25×10⁶ anti-CD19 CAR T cells.

### DETAILED DESCRIPTION

Provided herein are methods and uses of engineered cells (e.g., T cells) and/or compositions thereof, for the treatment of subjects having a disease or condition, which generally is or includes severe or moderate systemic autoimmune diseases. In some embodiments, the autoimmune disease is characterized by B cell involvement that is associated with and/or specific to cells expressing CD19. In particular embodiments of any of the provided methods and uses, the T cells are engineered with a chimeric antigen receptor (CAR) that is directed wholly or partially against cluster of differentiation 19 (CD19). Among such diseases for treatment with a CD19-directed CAR cell therapy are systemic autoimmune diseases including myasthenia gravis, Sjogren's syndrome or Primary Sjogren's disease, ANCA-associated vasculitis (AAV), rheumatoid arthritis, autoimmune encephalitis, pemphigus vulgaris, membranous nephropathy, IgG4-related diseases, neuromyelitis optica spectrum disorder (NMOSD), stiff-person syndrome (SPS), irritable bowel disease (IBD), thrombotic thrombocytopenia purpura (TTP), autoimmune hemolytic anemia (AIHA), immune thrombocytopenia (ITP), chronic immune thrombocytopenia (cITP), IgA nephropathy, bullous pemphigoid, progressive systemic sclerosis (i.e., scleroderma), idiopathic inflammatory myositis (IIM, including dermatomyositis, polymyositis and necrotizing myositis), mixed connective tissue disorder (MCTD), ulcerative colitis (UC), and relapsing-remitting multiple sclerosis.

CD19 is a member of the immunoglobulin superfamily and a component of the B-cell surface signal transduction complex that positively regulates signal transduction through the B-cell receptor. It is expressed by many B-cell malignancies from early development until differentiation into plasma cells (Stamenkovic et al., J Exp Med. 1988; 168(3):1205-10). CD19 is an attractive therapeutic target as CAR-T therapy has unique potential to provide transformational treatment for systemic autoimmune diseases with B cell involvement.

In particular, results herein demonstrate the advantageous effect that CD19-directed CAR T cells are able to induce an immune reset following targeted cytotoxic killing of CD19-expressing B cells. In some embodiments, as demonstrated in Example 2 in the context of relapsed or refractory (R/R) non-Hodgkin's lymphoma (NHL), compositions comprising the anti-CD19 CAR T cells are able to suppress B cell overactivation, resulting in an immune reset and the restoration of homeostatic immune system function. These results thus support use of CD19-directed CAR-expressing T cells to achieve the same effect to reset the immune system in autoimmune diseases by removal of the overactive B cells and to allow for reducing autoimmune disease activity and achieving clinical remission. Although other treatments such as use of HSCT or antibody therapies against B cell surface proteins have sought to deplete B cells or reset the immune system (e.g., Tyndall et al. Ann Rheum Dis 2001, 60:702-707; Sullivan et al. N Engl J Med 2018, 378:35-47; Wise and Stohl, Front. Med., 2020, &:303), none have been successful to efficiently decrease circulating B cells for reducing disease activity as observed herein by cytotoxic activity of CD19-directed CAR-expressing T cells and/or to do so while also minimizing toxicity to the subject from the therapy.

In embodiments of the provided methods, the therapeutic T cell compositions containing the engineered cells are administered to a subject having a severe or moderate autoimmune disease, e.g., via adoptive cell therapy, such as adoptive T cell therapy. In some aspects, the disease or condition is systemic autoimmune disease.

In particular embodiments, the subjects to be treated are a difficult to treat or high-risk group of subjects, including subjects that have relapsed or are refractory to one or more available prior therapies and/or who have severe disease. In some embodiments, the provided methods involve treating a specific group or subset of subjects, e.g., subjects identified as having high-risk disease, e.g., systemic autoimmune disease, such as severe systemic autoimmune or neurological disease. In some embodiments, subjects to be treated for the systemic autoimmune disease, such as any described herein, have relapsed or are refractory (R/R) to standard therapy for treating the systemic autoimmune or neurological disease and/or have a poor prognosis. In some aspects, the methods treat subjects having a severe disease that has relapsed or is refractory (R/R) to standard therapy or for which no approved therapy is available.

The genetically engineered T cells are generally administered in a composition formulated for administration; the methods generally involve administering one or more doses of the cells to the subject, which dose(s) may include a particular number or relative number of cells or of the engineered cells. In some cases, the CD19-directed CAR+engineered cells in the composition include a defined ratio or compositions of two or more sub-types within the composition, such as CD4+ vs. CD8+ T cells.

In some aspects, the methods and uses provide for or achieve improved response and/or more durable responses or efficacy and/or a reduced risk of toxicity or other side effects, e.g., in particular groups of subjects treated, as compared to certain alternative methods. In some aspects, the provided methods, compositions, uses and articles of manufacture achieve improved and superior responses to available therapies. In some embodiments, the improved or superior responses are to current standard of care (SOC). The CD19 CAR T cell therapy by the provided methods offers transformational efficacy and favorable safety profile in subjects with systemic autoimmune diseases or neurological diseases, particularly those with severe disease or who have relapsed/refractory disease.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods and Uses of Cd19-Targeted Cell Therapy in Autoimmune And Neurological Diseases

Provided herein are methods and use of CD19-directed CAR engineered cells (e.g., T cells) and/or compositions thereof, including methods for the treatment of subjects with systemic autoimmune diseases, including severe or moderate systemic autoimmune or neurological diseases that have failed one or more prior therapies, such as at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In particular embodiments, the method includes administering to the subject a dose of T cells that includes CD4+ and CD8+ T cells, wherein the T cells comprise a chimeric antigen receptor (CAR) that specifically binds to CD19 or specifically binds to CD19 and another antigen (e.g., BCMA, CD20, CD22, GPRC5D, ROR1). Cells engineered with such CARs or cell composition containing the same, are described in Section II.

In some embodiment, the methods provided herein are used to treat autoimmune diseases caused by, associated with and/or specific to cells expressing CD19, such as, systemic autoimmune and/or neurological diseases including myasthenia gravis, Sjogren's syndrome or Primary Sjogren's disease, ANCA-associated vasculitis (AAV), rheumatoid arthritis, autoimmune encephalitis, pemphigus vulgaris, membranous nephropathy, IgG4-related diseases, neuromyelitis optica spectrum disorder (NMOSD), stiff-person syndrome (SPS), irritable bowel disease (IBD), thrombotic thrombocytopenia purpura (TTP), autoimmune hemolytic anemia (AIHA), immune thrombocytopenia (ITP), chronic immune thrombocytopenia (cITP), IgA nephropathy, bullous pemphigoid, progressive systemic sclerosis (i.e., scleroderma), idiopathic inflammatory myositis (IIM, including dermatomyositis, polymyositis and necrotizing myositis), mixed connective tissue disorder (MCTD), ulcerative colitis (UC), and relapsing-remitting multiple sclerosis (MS).

In some embodiments, the methods provided herein are used to treat myasthenia gravis. In some embodiments, the systemic autoimmune disease is myasthenia gravis.

In some embodiments, the methods provided herein are used to treat Sjogren's syndrome or Primary Sjogren's disease. In some embodiments, the systemic autoimmune disease is Sjogren's syndrome or Primary Sjogren's disease.

In some embodiments, the methods provided herein are used to treat ANCA-associated vasculitis (AAV). In some embodiments, the systemic autoimmune disease is ANCA-associated vasculitis (AAV).

In some embodiments, the methods provided herein are used to treat rheumatoid arthritis. In some embodiments, the systemic autoimmune disease is rheumatoid arthritis.

In some embodiments, the methods provided herein are used to treat autoimmune encephalitis. In some embodiments, the systemic autoimmune disease is autoimmune encephalitis.

In some embodiments, the methods provided herein are used to treat pemphigus vulgaris. In some embodiments, the systemic autoimmune disease is pemphigus vulgaris.

In some embodiments, the methods provided herein are used to treat membranous nephropathy. In some embodiments, the systemic autoimmune disease is membranous nephropathy.

In some embodiments, the methods provided herein are used to treat IgG4-related diseases. In some embodiments, the systemic autoimmune disease is IgG4-related diseases.

In some embodiments, the methods provided herein are used to treat neuromyelitis optica spectrum disorder (NMOSD). In some embodiments, the systemic autoimmune disease is neuromyelitis optica spectrum disorder (NMOSD).

In some embodiments, the methods provided herein are used to treat stiff-person syndrome (SPS). In some embodiments, the systemic autoimmune disease is stiff-person syndrome (SPS).

In some embodiments, the methods provided herein are used to treat irritable bowel disease (IBD). In some embodiments, the systemic autoimmune disease is irritable bowel disease (IBD).

In some embodiments, the methods provided herein are used to treat thrombotic thrombocytopenia purpura (TTP). In some embodiments, the systemic autoimmune disease is thrombotic thrombocytopenia purpura (TTP).

In some embodiments, the methods provided herein are used to treat autoimmune hemolytic anemia (AIHA). In some embodiments, the systemic autoimmune disease is autoimmune hemolytic anemia (AIHA).

In some embodiments, the methods provided herein are used to treat immune thrombocytopenia (ITP). In some embodiments, the systemic autoimmune disease is immune thrombocytopenia (ITP).

In some embodiments, the methods provided herein are used to treat chronic immune thrombocytopenia (ITP). In some embodiments, the systemic autoimmune disease is chronic immune thrombocytopenia (ITP).

In some embodiments, the methods provided herein are used to treat IgA nephropathy. In some embodiments, the systemic autoimmune disease is IgA nephropathy.

In some embodiments, the methods provided herein are used to treat bullous pemphigoid. In some embodiments, the systemic autoimmune disease is bullous pemphigoid.

In some embodiments, the methods provided herein are used to treat ulcerative colitis (UC). In some embodiments, the systemic autoimmune disease is ulcerative colitis.

In some embodiments, the methods and uses include administering to the subject cells expressing genetically engineered (recombinant) cell surface receptors in adoptive cell therapy, which are chimeric antigen receptors (CARs) recognizing CD19. The cells are generally administered in a composition formulated for administration. In some embodiments, cells are collected from the subject prior to treatment for the purpose of engineering the cells with the CD19-directed recombinant receptor (e.g., CAR).

In some embodiments, the subject has received one or more prior therapies, such as two or more prior therapies, for treating the autoimmune disease or neurological disorder. In some embodiments, the subject has received 1 prior therapy for treating the systemic autoimmune disease or neurological disorder. In some embodiments, the subject has received 2 prior therapies for treating the systemic autoimmune disease or neurological disorder. In some embodiments, the subject has received 3 prior therapies for treating the systemic autoimmune disease or neurological disorder.

In some embodiments, the systemic autoimmune disease or neurological disorder is a refractory disease. In some embodiments, the refractory disease is characterized by an absence of response to one or more prior therapy, such as one or more standard therapy. In some embodiments, the refractory disease is characterized by an absence of a complete response to one or more prior therapies, such as to one or more standard therapy. In some embodiments, the subject is refractory to treatment with one or more prior therapy for treating the systemic autoimmune disease or neurological disorder. In some embodiments, the subject is refractory to treatment with two or more prior therapies for treating the systemic autoimmune disease or neurological disorder.

In some embodiments, the systemic autoimmune disease is a severe autoimmune or neurological disease. In some embodiments, the severe autoimmune or neurological disease is one in which the subject has achieved a response to a standard therapy, but the response is inadequate or partial. In some embodiments, the severe autoimmune or neurological disease is one in which a response in the subject is only achievable in the subject with a combination of standard therapy drugs.

In some embodiments, the one or more prior therapies, such as two or more prior therapies, is a standard therapy for treating the autoimmune or neurological disease. In some embodiments, the standard therapy is an anti-inflammatory drug, a steroid, such as a corticosteroid, a pain-killing medication (e.g., paracetamol or codeine), or an immunosuppressant drug, or combinations thereof.

In some embodiments, the subject has not previously received CAR T cell therapy prior to administration of the CD19-directed engineered CAR T cells in accord with the provided methods. In some embodiments, the subject has not received genetically-modified T cell therapy. In some embodiments, the subject has not received CD19-targeted therapy. Exemplary CD19-targeted therapies include, but are not limited to, anti-CD19 monoclonal antibodies or anti-CD19 bispecific antibodies. In some embodiments, the subject does not have hypersensitivity to fludarabine and/or cyclophosphamide.

In particular embodiments, prior to administration of the dose of CD19-directed engineered CAR T cells, the subject is administered or has received a lymphodepleting chemotherapy. Lymphodepletion may improve the engraftment and activity of CAR T cells through homeostatic cytokines, reduction of CD4+ CD25+regulatory T cells, increase of SDF-1 within bone marrow microenvironment, and stimulatory effects on antigen presenting cells (Grossman et al., Nat Rev Immunol. 2004; 4(5):387-395; Stachel et al., Pediatr Blood Cancer 2004; 43(6):644-50; Pinthus et al., J Clin Invest 2004; 114(12):1774-81; Turk et al., J Exp Med 2004; 200(6):771-82). In addition, LD chemotherapy may further lower the risk and severity of cytokine release syndrome (CRS).

Thus, in some embodiments, the methods include administering a preconditioning agent, such as a lymphodepleting or chemotherapeutic agent, such as cyclophosphamide, fludarabine, or combinations thereof, to a subject prior to the administration of engineered cells. For example, the subject may be administered a preconditioning agent at least 2 days prior, such as at least 3, 4, 5, 6, 7, 8, or 9 days prior, to the administration of engineered cells. In some embodiments, the subject is administered a preconditioning agent no more than 9 days prior, such as no more than 8, 7, 6, 5, 4, 3, or 2 days prior, to the administration of engineered cells.

In some embodiments, the subject is preconditioned with cyclophosphamide at a dose between or between about 20 mg/kg and 100 mg/kg body weight of the subject, such as between or between about 40 mg/kg and 80 mg/kg. In some aspects, the subject is preconditioned or administered with or with about 60 mg/kg of cyclophosphamide. In some embodiments, the cyclophosphamide may be administered in a single dose or may be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m² body surface area of the subject, such as between or between about 200 mg/m² and 400 mg/m², or 250 mg/m² and 350 mg/m², inclusive. In some instances, the subject is administered about 100 mg/m² of cyclophosphamide. In some instances, the subject is administered about 150 mg/m² of cyclophosphamide. In some instances, the subject is administered about 200 mg/m² of cyclophosphamide. In some instances, the subject is administered about 250 mg/m² of cyclophosphamide. In some instances, the subject is administered about 300 mg/m² of cyclophosphamide. In some embodiments, the cyclophosphamide may be administered in a single dose or may be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² body surface area of the subject, of cyclophosphamide, daily for 3 days, prior to initiation of the cell therapy. In some embodiments, the subject is administered a total of at or about 300 mg/m², 400 mg/m², 500 mg/m², 600 mg/m², 700 mg/m², 800 mg/m², 900 mg/m², 1000 mg/m², 1200 mg/m², 1500 mg/m², 1800 mg/m², 2000 mg/m², 2500 mg/m², 2700 mg/m², 3000 mg/m², 3300 mg/m², 3600 mg/m², 4000 mg/m² or 5000 mg/m² cyclophosphamide, or a range defined by any of the foregoing, prior to initiation of the cell therapy.

In some embodiments, where the lymphodepleting agent comprises fludarabine, the subject is administered fludarabine at a dose between at or about 1 mg/m² and at or 100 mg/m², such as between at or about 10 mg/m² and at or about 75 mg/m², at or about 15 mg/m² and at or about 50 mg/m², at or about 20 mg/m² and at or about 40 mg/m², at or about or 24 mg/m² and at or about 35 mg/m², inclusive. In some instances, the subject is administered at or at or about 10 mg/m² of fludarabine. In some instances, the subject is administered at or about 15 mg/m² of fludarabine. In some instances, the subject is administered at or about 20 mg/m² of fludarabine. In some instances, the subject is administered at or about 25 mg/m² of fludarabine. In some instances, the subject is administered at or about 30 mg/m² of fludarabine. In some embodiments, the fludarabine may be administered in a single dose or may be administered in a plurality of doses, such as given daily, every other day or every three days. In some embodiments, fludarabine is administered daily, such as for 1-5 days, for example, for 3 to 5 days. In some instances, the subject is administered at or about 30 mg/m² body surface area of the subject, of fludarabine, daily for 3 days, prior to initiation of the cell therapy. In some embodiments, the subject is administered a total of at or about 10 mg/m², 20 mg/m², 25 mg/m², 30 mg/m², 40 mg/m², 50 mg/m², 60 mg/m², 70 mg/m², 80 mg/m², 90 mg/m², 100 mg/m², 120 mg/m², 150 mg/m², 180 mg/m², 200 mg/m², 250 mg/m², 270 mg/m², 300 mg/m², 330 mg/m², 360 mg/m², 400 mg/m² or 500 mg/m² cyclophosphamide, or a range defined by any of the foregoing, prior to initiation of the cell therapy.

In some embodiments, the lymphodepleting agent comprises a single agent, such as cyclophosphamide or fludarabine. In some embodiments, the subject is administered cyclophosphamide only, without fludarabine or other lymphodepleting agents. In some embodiments, prior to the administration, the subject has received a lymphodepleting therapy comprising the administration of cyclophosphamide at or about 200-400 mg/m² body surface area of the subject, optionally at or about 300 mg/m², daily, for 2-4 days. In some embodiments, the subject is administered fludarabine only, for example, without cyclophosphamide or other lymphodepleting agents. In some embodiments, prior to the administration, the subject has received a lymphodepleting therapy comprising the administration of fludarabine at or about 20-40 mg/m² body surface area of the subject, optionally at or about 30 mg/m², daily, for 2-4 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered at or about 60 mg/kg (˜2 g/m²) of cyclophosphamide and 3 to 5 doses of 25 mg/m² fludarabine prior to the first or subsequent dose. In some aspects, the subject is administered fludarabine (30 mg/m²/day for 3 days) and cyclophosphamide (300 mg/m²/day for 3 days) (flu/cy) concurrently, intravenously, prior to administration of the cells. In some embodiments, the subject is administered a reduced, delayed or eliminated dose of one or more doses of the lymphodepleting agent(s).

In some embodiments, the subjects are premedicated, e.g., to minimize the risk of infusion reaction. In some aspects, the premedication includes administering pain reliever and/or an antihistamine. In some embodiments, the premedication includes administering an acetaminophen and/or a diphenhydramine, or another H1-antihistamine. In some embodiments, the patient with acetaminophen (e.g., 650 mg orally) and diphenhydramine (e.g., 25-50 mg, IV or orally), or another H1-antihistamine, at or about 30 to 60 minutes prior to treatment with the cell therapy.

In embodiments of any of the provided methods, the subject is a human subject.

### A. Exemplary Diseases

### 1. Myasthenia Gravis (MG)

Myasthenia gravis (MG) is an antibody-mediated autoimmune disorder affecting the neuromuscular junction (NMJ). MG is caused by antibodies against the acetylcholine receptor (AChR), muscle-specific kinase (MuSK), or other AChR-related proteins in the postsynaptic muscle membrane. Localized or general muscle weakness is the predominant symptom and is induced by the antibodies. Patients are grouped according to the presence of antibodies, symptoms, age at onset, and thymus pathology. Myasthenia gravis may cause a significant number of complications. These include myasthenic crisis, an acute respiratory paralysis that requires intensive care, as well as adverse events due to long term medication treatment like opportunistic infections and lymphoproliferative malignancies. See, e.g., Gilhus, N. E., Tzartos, S., Evoli, A. et al. Myasthenia gravis. Nat Rev Dis Primers 5, 30 (2019) and Beloor Suresh A, Asuncion R M D. Myasthenia Gravis. [Updated 2023 Aug 8]. In: StatPearls. Treasure Island (FL): StatPearls Publishing; 2024 Jan.

The pathophysiologic mechanisms in MG are dependent on the type of antibodies present. In n-AChR MG, the antibodies are of the IgG1 and IgG3 subtype. They bind to the n-ACh receptor present in the postsynaptic membrane of the skeletal muscles and activate the complement system leading to the formation of the membrane attack complex (MAC). MAC brings about the final degradation of the receptors. They may also act by functionally blocking the binding of ACh to its receptor or by enhancing the endocytosis of the antibody-bound n-ACh receptor. In MusK MG and LPR4 MG, the antibodies are of the IgG4 subtype and do not have the complement activating property. They bind to the Agrin-LRP4-MuSK protein complex in the NMJ, whose primary function is the maintenance of the NMJ, including the n-ACh receptor distribution and clustering. The inhibition of the complex leads to a reduced number of n-ACh receptors. The ACh released at the nerve terminal, in turn, is unable to generate the postsynaptic potential required to generate an action potential in muscle due to a reduced number of n-ACh receptors leading to the symptoms of muscle weakness. The weakness is more pronounced with the repeated use of a muscle group since it causes depletion of the ACh store in the NMJ.

Clinical features at the onset and during the evolution of MG include but are not limited to fluctuating muscle weakness that varies in severity, worsens with physical activity, and improves with rest, extraocular muscle weakness, diplopia or ptosis or both, bulbar muscle weakness, limb weakness, myasthenic crisis, and widespread muscle weakness, including respiratory insufficiency producing respiratory failure. It has been noted that MuSK MG is more common in females, relatively spares extraocular muscles, and commonly involves bulbar, facial, and neck muscles. Myasthenic crisis is also frequent in the MuSK MG. Further, despite resolution of manifest weakness with treatment, many patients complain of general fatigue, as assessed by patient-reported outcome measures and patient survey. See, e.g., Kaminski, Henry J et al. “Myasthenia gravis: the future is here.” The Journal of clinical investigation vol. 134,12 e179742. 17 Jun. 2024.

The Myasthenia Gravis Foundation of America (MGFA) clinical classification divides MG into 5 main classes based on the clinical features and disease severity. Class I: involves any ocular muscle weakness, including weakness of eye closure and all other muscle groups are normal. Class II: involves mild weakness of muscles other than ocular muscles, and ocular muscle weakness of any severity may be present. Class IIa: involves predominant weakness of the limb, axial muscles, or both, and it may also involve the oropharyngeal muscles to a lesser extent. Class IIb: involves oropharyngeal, respiratory muscles, or both, and it may have the involvement of limb, axial muscles, or both to a lesser extent. Class III: involves muscles other than ocular muscles moderately, and ocular muscle weakness of any severity may be present. Class IIIa: involves the limb, axial muscles, or both predominantly, and oropharyngeal muscles may be involved to a lesser degree. Class IIIb: involves oropharyngeal, respiratory muscles, or both predominantly, and the limb, axial muscles, or both may have a lesser or equal involvement. Class IV: involves severe weakness of affected muscles, and ocular muscle weakness of any severity may be present. Class IVa: involves limb, axial muscles, or both predominantly, and oropharyngeal muscles may be involved to a lesser degree. Class IVb: involves oropharyngeal, respiratory muscles, or both predominantly, and the limb, axial muscles, or both may have lesser or equal involvement, and also includes patients requiring feeding tubes without intubation. Class V: involves intubation with or without mechanical ventilation, except when employed during routine postoperative management.

Patient-reported outcome measures (PROMs) may be used to evaluate MG and other chronic inflammatory diseases. MG-specific PROMs, such as MG activities of daily living (MG-ADL), more readily reflect the disease activity over time compared to point-in-time evaluations, which is particularly important in a disease like MG, given its known fluctuation during the day. A cross-sectional prevalence cohort study reported almost half of the population (47%) reported MG-ADL>3p, corresponding to an unsatisfactory symptom state, indicating the need for improved treatment options. See, e.g., Petersson, Malin et al. “Patient-Reported Symptom Severity in a Nationwide Myasthenia Gravis Cohort: Cross-sectional Analysis of the Swedish GEMG Study.” Neurology vol. 97,14 e1382-e1391. 4 Oct. 2021.

MG may be diagnosed using clinical assessments including serologic anti-AChR Ab test, repetitive nerve stimulation (RNS) test and single-fiber electromyography (SFEMG) to assess conduction delays in the NMJ, edrophonium (Tensilon) test that increases the availability of ACh in the NMJ, ice-pack test, imaging (e.g., Chest computed tomography (CT) or magnetic resonance imaging (MRI)), and other laboratory tests, as myasthenia gravis commonly coexists with other autoimmune disorders, and testing for anti-nuclear (ANA) antibodies, rheumatoid factor (RF), and baseline thyroid functions is recommended.

Treatment is based on MG subgroup and includes symptomatic treatment using acetylcholinesterase inhibitors, thymectomy, immunosuppressive drugs, and immunotherapy. Intravenous immunoglobulin and plasma exchange are fast-acting treatments used for disease exacerbations, and intensive care is necessary during exacerbations with respiratory failure. Comorbidity is frequent, particularly in elderly patients.

Surgical therapy comprises thymectomy for patients with early-onset AChR antibody-positive MG, however, up to one-quarter of patients respond poorly and continue to require doses of prednisone and immunosuppressives. The choice of treatment of pharmacological therapy may be influenced by several factors, including severity of disease, the patient's individual characteristics, and the presence of comorbid conditions. Furthermore, although prednisone is still consistently effective drug for MG, its side effect burden and that of immunosuppressive as well as the poor response in a large minority has motivated development of new therapies.

Treatments also include inhibition of the neonatal Fc receptor (FcRn), as antibodies in circulation bind to FcRn and are internalized, ultimately entering lysosomes, but are normally recycled back into circulation. FcRn inhibitors disrupt this binding within the lysosome, leading to the proteolytic removal of antibodies generally and including the subset of disease-causing antibodies. This results in significant reductions in circulating antibodies within days of the initial treatment. Efgartigimod and rozanolixizumab are approved for AChR antibody-positive MG, with the latter further approved for MuSK antibody-positive MG. Trials of efgartigimod and rozanolixizumab in MG reported a subset of patients who responded poorly despite a reduction in circulating antibodies.

Furthermore, treatments also include inhibition of complement activation, which focus on targeting the C5 convertase enzyme, for example, eculizumab is a humanized chimeric monoclonal antibody designed to block cleavage of C5, and zilucoplan is a small macrocyclic peptide that binds to C5 to inhibit its cleavage. There remain upward of thirty percent of patients who do not benefit from complement inhibition, which demonstrates the importance of other mechanisms of autoantibody action. An important concern with all complement inhibitors is enhanced risk of meningococcal and encapsulated bacterial infections.

B-cell targeting is another area of therapeutic development for MG. A phase II study of rituximab, a chimeric antibody directed toward CD20 on B cells, in treatment-resistant MG did not achieve its primary outcome, while a phase III trial using rituximab within one year of disease onset improved clinical status. CD38 is expressed on plasma, NK, and T cells and was targeted by TAK-079 in a phase II trial that showed promising safety results (clinical trial: NCT04159805).

All of the therapies currently used for the treatment of MG have well known adverse effect profiles and there is a medical need to identify new targeted therapies, particularly agents that may reduce the requirement for corticosteroids and non-specific cytotoxic agents. First-line therapies include pyridostigmine, prednisone, and thymectomy. Second-line therapies include azathioprine, cyclosporine, and intravenous immunoglobulin. Third-line therapies include methotrexate, mycophenolate mofetil, and plasmapheresis. Fourth-line therapies include Rituximab, which is a chimeric anti-CD20 monoclonal antibody. Rituximab is an effective treatment in a number of autoimmune diseases, including rheumatoid arthritis and ANCA vasculitis. Fifth-line therapies include eculizumab and cyclophosphamide. See, e.g., Farmakidis, Constantine et al. “Treatment of Myasthenia Gravis.” Neurologic clinics vol. 36,2 (2018): 311-337.

Many subjects with MG, including severe MG, exhibit an insufficient response or are refractory to existing treatments, such as treatments with any two or more of the following: pyridostigmine, prednisone, thymectomy, azathioprine, cyclosporine, intravenous immunoglobulin, methotrexate, mycophenolate mofetil, plasmapheresis, Rituximab, eculizumab and cyclophosphamide. In some embodiments, the subject is refractory to treatment with two or more prior treatments. In some embodiments, the two or more prior treatments (e.g., 2, 3, 4, 5 or more prior treatments) are selected from any two or more of the following: pyridostigmine, prednisone, thymectomy, azathioprine, cyclosporine, intravenous immunoglobulin, methotrexate, mycophenolate mofetil, plasmapheresis, Rituximab, eculizumab and cyclophosphamide. It is estimated that of all patients with MG, a fraction (estimated at 10%) has MG disease that is refractory to treatment with conventional agents such as cholinesterase inhibitors and immunosuppressive agents (including corticosteroids, azathioprine, and cyclosporine). There is an unmet need for an MG therapy with a better efficacy and safety profile than currently available therapies, particularly in subjects with severe, refractory MG. See, e.g., Mantegazza, Renato, and Carlo Antozzi. “When myasthenia gravis is deemed refractory: clinical signposts and treatment strategies.” Therapeutic advances in neurological disorders vol. 11 1756285617749134. 18 Jan. 2018.

In some embodiments, methods include selection of subjects with symptoms indicative of class I, class II, class III, class IV, or class V MG. In some embodiments, methods include selection of subjects with symptoms indicative of class IV or class V MG. In some embodiments, methods include selection of subjects with symptoms indicative of class V MG. In some embodiments, methods include selection of subjects with symptoms indicative of treatment-refractory MG. While there is no broadly accepted consensus-based definition of ‘treatment-refractory MG’, the following attributes may be used to define treatment-refractory MG: failure to respond adequately to conventional therapies, inability to reduce immunosuppressive therapy without clinical relapse or a need for ongoing rescue therapy such as intravenous immunoglobulin G (IVIg) or plasma exchange (PE), severe or intolerable adverse effects from immunosuppressive therapy, comorbid conditions that restrict the use of conventional therapies, and/or frequent myasthenic crises even while on therapy. In some embodiments, methods include selection of subjects with symptoms indicative of treatment-refractory MG, including having one or more of the following attributes, anti-AChR positive, anti-MuSK positive, and thymectomy.

In some embodiments, the subject has an insufficient response to two prior treatments. In some embodiments, the subject has an insufficient response to three prior treatments. In some embodiments, the subject has an insufficient response to four or more prior treatments. In some embodiments, the subject has failed to attain clinical remission (e.g., after three months of a given treatment) after having been treated with any two or more prior treatments for MG. In any embodiments, the subject is identified or selected as having an insufficient response to a prior treatment at a time prior to leukapheresis in connection with engineering the CD19-directed CAR T cell composition. Insufficient response to treatments is defined as a lack of response, insufficient response, or a lack of sustained response to appropriate doses. Intolerance is not considered an insufficient response.

In some embodiments, the systemic autoimmune disease is MG, such as a moderate class III or severe class IV/V MG. Among provided methods are methods of treatment that involve administering engineered cells or compositions comprising engineered cells, such as engineered T cells to subjects with MG, including severe MG. Also provided are methods and uses of provided CD19-directed CAR engineered cells (e.g., T cells) and/or compositions thereof, including methods for the treatment of subjects having MG, including severe MG, that involves administration of the engineered cells and/or compositions thereof. In certain embodiments, the subject has severe MG. In some embodiments, the subject is selected for or identified as having severe MG, such as by the presence of certain features or clinical manifestations that indicate the presence of severe MG. In some embodiments, the subject has a high MG-ADL score. In some embodiments, the subject is anti-ChR, anti-LRP4, or anti-MuSK positive. In some embodiments, the subject has failed to achieve complete remission after receiving one or more prior therapy. In some embodiments, the subject is refractory to one or two prior lines of therapy. In some embodiments, the subject has an insufficient response to four or more prior lines of therapy. In some embodiments, the subject has an insufficient response to four or more prior lines of therapy and has severe refractory disease. Exemplary selection criteria are further described herein. In some embodiments, the methods and use of provided CD19-directed CAR engineered cells (e.g., T cells) and/or compositions thereof, include methods for the treatment of subjects with severe MG that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. a. Response and Efficacy

In some embodiments, the provided methods and uses involving administration of an anti-CD19 CAR T cell therapy reduce MG disease activity in the subject. In some embodiments, the anti-CD19 CAR T cell therapy reduces MG symptoms and may be useful in reducing or eliminating the need for prolonged corticosteroid therapy. In some embodiments, the primary efficacy endpoint is a change in the baseline Myasthenia Gravis Activities of Daily Living (MG-ADL) total score, which may be compared using a worst-rank score analysis. In some embodiments, the efficacy of anti-CD19 CAR T cell therapy is measured using Quantitative Myasthenia Gravis (QMG), which is a scoring system is considered to be an objective evaluation of muscle strength based on quantitative testing of sentinel muscle groups. The MGFA task force has recommended that the QMG score be used in prospective studies of therapy for MG. The QMG scoring system consists of 13 items. Each item is graded 0 to 3, with 3 being the most severe. The range of total QMG score is 0-39. In some embodiments, the efficacy endpoint is the percentage of patients with a 3-point reduction from baseline in the QMG Total Score for Disease Severity. In some embodiments, the treatment results in the subject achieving a 3-point reduction from baseline in the QMG Total Score for Disease Severity. In some embodiments, the treatment results in the subject having significant change in mean change from baseline in QMG total score.

In some embodiments, the treatment results in the subject achieving a negative QMG item score. The QMG scoring system is considered to be an objective evaluation of muscle strength based on quantitative testing of sentinel muscle groups. All individual QMG items are scored 0 to 3, with 3 being the most severe. Negative values imply an improvement in QMG Item Score.

In some embodiments, the treatment results in the subject achieving improvement in respiratory function tests to characterize the degree of involvement of respiratory muscles. In some embodiments, the treatment results in the subject achieving improvement in Forced Vital Capacity. In some embodiments, the treatment results in the subject achieving improvement in Negative Inspiratory Force (NIF). NIF is a measurement of respiratory muscle strength and ventilator reserve. NIF is represented by centimeters of water pressure (cmH2O). A normal NIF measurement is negative 60 cmH2O, or as 100% predicted value.

In some embodiments, the treatment results in the subject achieving improved MGFA post-intervention status (PIS). In some embodiments, the treatment results in the subject achieving unchanged in MGFA post-intervention status (PIS). Change in status categories of improved, unchanged, worse, exacerbation, and died of MG may be assessed and recorded after treatment of selected subjects with anti-CD19 CAR T cell therapy.

The MG-ADL is an 8-point questionnaire that focuses on relevant symptoms and functional performance of activities of daily living (ADL) in MG patients. The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). In some embodiments, the treatment results in the subject having significant change in the MG-ADL grade. In some embodiments, the treatment results in the subject having normal MG-ADL status. In some embodiments, the treatment results in the subject having a positive change from baseline in the MG-Activity of Daily Living profile (MG-ADL) status.

The SF-36 is a multi-purpose, short-form health survey with 36 questions. It yields an 8-scale profile of functional health and well-being scores (physical functioning, role-physical, bodily pain, general health, mental health, role-emotional, social functioning and vitality) as well as psychometrically-based physical and mental health summary measures. It is a generic measure, as opposed to one that targets a specific age, disease or treatment group. The lower the score the more disability; the higher the score the less disability. In some embodiments, the treatment results in the subject having a significant improvement in SF-36 measure.

In some embodiments, the treatment in accord with the provided methods results in clinical remission of MG in the subject that is maintained for greater than 3 months. In some embodiments, the treatment in accord with the provided methods results in clinical remission of MG in the subject that is maintained for greater than 4 months, 5 months, 6 months, 7 months, 8 months, 9 months, 10 months, 11 months, 12 months, 18 months, 24 months, 3 years, 4 years, 5 years or more. In some embodiments, the treatment in accord with the provided methods results in clinical remission of MG in the subject that is maintained for greater than 6 months. In some embodiments, the treatment in accord with the provided methods results in clinical remission of MG in the subject that is maintained for greater than 12 months. In some embodiments, the treatment in accord with the provided methods results in clinical remission of MG in the subject that is maintained for greater than 24 months. In some embodiments, the treatment in accord with the provided methods results in clinical remission of MG in the subject that is maintained for greater than 3 years. In some embodiments, the treatment in accord with the provided methods results in clinical remission of MG in the subject that is maintained for greater than 4 years. In some embodiments, the treatment in accord with the provided methods results in clinical remission of MG in the subject that is maintained for greater than 5 years.

In some embodiments, the treatment in accord with the provided methods results in prolonged remission. In some embodiments, prolonged remission is defined as a 5-year consecutive period of no disease activity and without treatment (e.g., corticosteroids, antimalarials, or immunosuppressants).

### 2. Primary Sjogren's Disease (SjD)

Primary Sjogren's Disease (SjD), also known as Primary Sjogren syndrome or Sjogren's syndrome, is a systemic autoimmune disorder commonly presenting with sicca symptoms, which refers to dryness involving the eyes and mouth due to inflammation and resultant pathology of the lacrimal and salivary glands. Up to one-half of affected individuals also develop extra-glandular involvement in organs such as the joints, skin, lungs, gastrointestinal (GI) tract, nervous system, and kidneys. SjD may also cause systemic manifestations such as joint pain, swelling, and fatigue, and may lead to dental and corneal complications (e.g., oral candidiasis). SjD patients can also develop arthritis and arthralgia. SjD may fluctuate in severity and organ involvement. Patients often experience a variable course with periods of increased symptoms, commonly referred to as flare. Extra-glandular involvement significantly worsens prognosis. Further, studies show African Americans may be disproportionately afflicted with moderate-severe disease.

Different clinical associations have been described for each of the diverse autoantibodies found in SjD patients. Antibodies directed against the Ro/La ribonucleoprotein complexes have been correlated with younger age, more severe dysfunction of the exocrine glands and a higher prevalence of extraglandular manifestations. The disorder occurs either in a primary form or in association with other autoimmune conditions, more commonly rheumatoid arthritis (RA), systemic lupus erythematosus (SLE) and systemic sclerosis (SSc). SjD subjects may also develop fatal comorbidities, including Non-Hodgkin Lymphoma, severe neurological impairment (e.g., neuropathy, trigeminal neuralgia, and mononeuritis), and severe respiratory issues (e.g., interstitial lung disease). For example, strong predictors of B-cell lymphoma include: salivary gland enlargement, lymphadenopathy, Raynaud's, anti-Ro/SSA or/and anti-La/SSB, RF positivity, monoclonal gammopathy, and C4 hypocomplementemia. See, e.g., Bournia, Vasiliki-Kalliopi, and Panayiotis G Vlachoyiannopoulos “Subgroups of Sjogren syndrome patients according to serological profiles,” Journal of autoimmunity vol. 39,1-2 (2012): 15-26.

The characteristic lesion of Sjogren's syndrome is focal lymphocytic sialadenitis (FLS). FLS is a lesion of exocrine glands. Foci of lymphocyte-rich mononuclear cells infiltrate exocrine glandular tissue adjacent to blood vessels and excretory ducts. The foci are comprised predominantly of T lymphocytes. However, B lymphocytes, plasma cells, and other cell types are seen. With more severe disease the foci may become confluent. The infiltrating mononuclear cells, humoral factors such as antibodies and cytokines, or both are hypothesized to cause exocrine gland dysfunction resulting in diminished tear production by the lacrimal glands and diminished saliva production by salivary glands. See, e.g., Carsons S E, Patel B C. Sjogren Syndrome. [Updated 2023 Jul 31]. In: StatPearls. Treasure Island (FL): StatPearls Publishing; 2024 Jan.

In some embodiments, subjects display extraglandular manifestations of SjD. In some embodiments, these extraglandular manifestations may include one or more of the following manifestations, skin dryness, hair loss (telo-effluvium), and scarring alopecia, maculopapular rashes, leukocytoclastic vasculitis, urticaria and urticarial vasculitis, Raynaud's phenomena, digital ulceration, and acrocyanosis, infectious (including Herpes zoster), embolic and thrombotic lesions, arthralgia/arthritis including overlap syndromes with rheumatoid arthritis, SLE, Jaccoud's arthritis, osteoarthritis, erosive osteoarthritis, and seronegative spondyloarthropathies, myalgias and myositis including overlap with SLE, polymyositis, inclusion body myositis, metabolic myopathies, and neuropathic myopathies; overlaps with myositis, fibromyalgia, thyroiditis, diabetes, adrenal insufficiency including autoimmune and catastrophic cardiolipin syndrome, androgen/estrogen replacement, autonomic neuropathy, interstitial pneumonitis, pleurisy and pleural effusions including lymphomatous, pulmonary hypertension and occult pulmonary emboli, lymphoproliferative manifestations (BALT (bronchial MALT lymphoma)), laryngotracheal reflux and motility disorders, aspiration pneumonia in the SS patient with dysphagia, autoimmune hepatitis and biliary cirrhosis, and pancreatitis. See, e.g., Fox, Robert I. “Extraglandular Manifestations of Sjogren's Syndrome (SS): Dermatologic, Arthritic, Endocrine, Pulmonary, Cardiovascular, Gastroenterology, Renal, Urology, and Gynecologic Manifestations.” Sjogren's Syndrome: Practical Guidelines to Diagnosis and Therapy 285-316. 12 Apr. 2011.

The mechanism underlying the development of SjD is the destruction of the epithelium of the exocrine glands, as a consequence of abnormal B cell and T cell responses to the autoantigens Ro/SSA and La/SSB, among others. Diagnostic criteria for SjD include the detection of autoantibodies in patient serum and histological analysis of biopsied salivary gland tissue. Sjagren syndrome is characterized by a variety of autoantibodies, both organ specific and non-specific. The anti-SSA/Ro and anti-SSB/La antibodies are considered typical among them and have been included in the American-European Consensus Group classification criteria. To evaluate for SjD, tests performed may include a Schirmer test, slit-lamp exam with vital dye staining, salivary flow rate, and/or nuclear scintigraphic evaluation of the salivary glandular function. Assessment of autoantibodies (ANA, RF, SS-A, and SS-B) is also performed. While dual seropositivity (e.g., SS-A and SS-B) is less common, it is widely thought to be an accurate predictor of severe disease.

In some embodiments, methods include selection of subjects with symptoms indicative of SjD. In some embodiments, methods include selection of subjects using ACR-EULAR Classification Criteria for primary Sjogren's syndrome. In some embodiments, methods include selection of subjects with the inclusion criteria using ACR-EULAR. In some embodiments, methods include selection of subjects using ocular symptoms, oral symptoms, ocular signs, histopathology, oral signs, and/or the presence of autoantibodies to make the determination the subject has primary Sjogren's disease. In some embodiments, the ocular signs comprise at least an abnormal Schirmer's test, (without anesthesia; ≤5 mm/5 minutes) and/or a positive vital dye staining of the eye surface. In some embodiments, histopathology shows a lip biopsy showing focal lymphocytic sialadenitis (focus score≥1 per 4 mm2). In some embodiments, the ocular signs comprise at least unstimulated whole salivary flow (≤1.5 mL in 15 minutes), an abnormal parotid sialography, and/or an abnormal salivary scintigraphy. Imaging tests of salivary glands may also be performed for diagnosis. In some embodiments, the subjects are positive for anti-SSA (Ro), anti-SSB (La), or both. In some embodiments, methods include selection of subjects with any four of the six criteria. In some embodiments, methods include selection of subjects that must include histopathology or the determination of presence of autoantibodies. See, e.g., Shiboski, Caroline H et al. “2016 American College of Rheumatology/European League Against Rheumatism Classification Criteria for Primary Sjagren's Syndrome: A Consensus and Data-Driven Methodology Involving Three International Patient Cohorts.” Arthritis & rheumatology (Hoboken, N.J.) vol. 69,1 (2017): 35-45.

SjD symptoms may further include dry eyes (xerophthalmia) leading to irritation, a gritty sensation, or burning, dry mouth (xerostomia) causing difficulty in swallowing, speaking, and tasting, persistent dry cough, joint pain and swelling resembling rheumatoid arthritis (RA), swelling of salivary glands, fatigue and chronic tiredness, skin, nose, and vaginal dryness.

Therapeutic approaches for SjD include both topical and systemic treatments to manage the sicca and systemic symptoms of disease. SjD is a serious disease with excess mortality, mainly related to the systemic involvement of disease and the development of lymphomas in some patients. Knowledge of SjD has progressed substantially, but this disease is still characterized by sicca symptoms, the systemic involvement of disease, lymphocytic infiltration to exocrine glands, the presence of anti-Ro/SSA and anti-La/SSB autoantibodies and the increased risk of lymphoma in patients with SjD. Current treatment options are largely ineffective, as these options lack targeted therapies and borrow therapies from adjacent indications. As symptoms deeply affect quality of life there is an unmet need for SjD therapeutics. While treatment is mostly symptomatic in glandular onset, the manifestation in other organs often hinders patient's quality of life (e.g., difficult to breath, arthralgia, Raynaud's, interstitial lung disease, decreased cognitive dysfunction as implication of neurological onset, etc.).

In some embodiments, methods include treatment of subjects with refractory SjD. Current treatment programs for refractory SjD do not comprise disease modifying treatments and include long-term use of biologics and corticosteroids to control flares. There is an unmet need in the treatment program for highly symptomatic SjD patients. These subjects experience an initial onset of mucosal dryness, progression of disease in several organs (e.g., breathing difficulty, joint stiffness/swelling, blindness, impacted lymph nodes, kidney disease, and liver disease), and 3-10% of these patients progress to Non-Hodgkin Lymphoma. In some embodiments, subjects with refractory SjD have a high ESSDAI score, are SSA and SSB positive, have severe extraglandular disease, and have received three or more prior lines of therapy. In some embodiments, the subject has an insufficient response to three or more prior lines of therapy and has severe refractory disease.

Current treatments comprise eye drops, eye ointments, artificial saliva, anti-fungal medications, disease-modifying anti-rheumatic drugs (DMARDs) and anti-malarial drugs. The first approach to extra-glandular (systemic) major organ-system disease is oral/parenteral corticosteroids. Corticosteroids may also be used for moderate to severe cases. Immunosuppressive drugs may be used to treat systemic symptoms, and hydroxychloroquine may be used for musculoskeletal pain. Biologics targeting B cells and the BLyS/BAFF pathway may also be used to treat SjD. Rituximab is an option for patients with keratoconjunctivitis sicca, vasculitis, xerostomia and severe parotid gland swelling. All patients on Rituximab must be closely monitored for tumor lysis syndrome (when patients have lymphoma), cytopenia, infusion reactions, hepatitis B reactivation and serious fungal, viral and bacterial infections. With currently no FDA approved therapies for SjD, variable success with Rituximab, and very few options available for refractory patients there is a significant unmet need.

In some embodiments, the subjects are diagnosed with the systemic autoimmune disease SjD. In some embodiments, the subjects with SjD have extraglandular manifestations. In some embodiments, the subjects with SjD have severe extraglandular manifestations, including interstitial lung disease (ILD), peripheral nervous system (PNS) manifestations, and renal disease. In some embodiments, methods include selection of subjects with symptoms indicative of SjD with extraglandular manifestations. In some embodiments, methods include selection of subjects with symptoms indicative of SjD with glandular manifestations. In some embodiments, methods include selection of subjects with symptoms indicative of refractory or relapsed SjD. In some embodiments, the subjects are positive for anti-SSA (Ro), anti-SSB (La), or both. In some embodiments, the subjects have a high EULAR SS Disease Activity Index (ESSDAI) score. In some embodiments, the subjects have an ESSDAI score greater than or equal to 14. In some embodiments, the subjects are refractory to three or more lines of treatment and exhibit extraglandular disease manifestations. For example, these subjects may have failed multiple immunotherapies and have developed ILD or severe renal or PNS involvement. In some embodiments, the subjects are refractory to three or more lines of treatment, have a high ESSDAI score, and are SS-A and/or SS-B positive. For example, the subject may have failed three or more immunotherapies, is SSA+ and/or SSB+, and has high disease severity. In some embodiments, the subjects exhibit extraglandular disease manifestations. For example, the subject has received a limited number of therapies, but presents with extraglandular involvement requiring aggressive treatment quickly. Among provided methods are methods of treatment that involve administering engineered cells or compositions comprising engineered cells, such as engineered T cells to subjects with SjD, including severe SjD. Also provided are methods and uses of provided CD19-directed CAR engineered cells (e.g., T cells) and/or compositions thereof, including methods for the treatment of subjects having SjD, including severe SjD, that involves administration of the engineered cells and/or compositions thereof. In certain embodiments, the subject has severe SjD. In some embodiments, the subject is selected for or identified as having severe SjD, such as by the presence of certain features or clinical manifestations that indicate the presence of severe SjD. Exemplary selection criteria are further described herein. In some embodiments, the methods and use of provided CD19-directed CAR engineered cells (e.g., T cells) and/or compositions thereof, include methods for the treatment of subjects with severe SjD that have failed at least one or more prior therapies. In particular embodiments, the method includes administering to the subject a dose of T cells that includes CD4+ and CD8+ T cells, wherein the T cells comprise a chimeric antigen receptor (CAR) that specifically binds to CD19. a. Response and Efficacy

In some embodiments, a subject who has been treated in accord with the provided methods is evaluated or monitored after treatment for a period of time to determine whether a complete or partial remission has occurred. In some embodiments, the subject is evaluated or monitored to assess whether the remission achieved according to the measurement is being maintained.

In some embodiments, the provided methods and uses involving administration of an anti-CD19 CAR T cell therapy reduce SjD disease activity in the subject.

Disease activity indices for SjD endpoints have recently been developed and validated by the EULAR SjOgren's Task Force: EULAR SS Patient Reported Index (ESSPRI) and EULAR SS Disease Activity Index (ESSDAI). ESSPRI is a patient-administered questionnaire assessing patient's subjective symptoms, and ESSDAI is a clinical index that measures systemic disease activity. ESSPRI questionnaire includes three different 10-point Likert scales and patients' answers may range from 0 (absence of symptom) to 10 (maximum imaginable intensity). The ESSDAI includes 12 domains that represent the manifestations across organs (constitutional, lymphadenopathy, glandular, articular, cutaneous, pulmonary, renal, muscular, peripheral nervous system, central nervous system, hematological and biological), divided into 3-4 levels of activity, and the score range is 0-123. Higher scores on the EULAR Sjogren's Syndrome Disease Activity Index (ESSDAI) scale are associated with poorer health states. High disease activity has score≥14 and is characterized by multiple organ involvement. A negative change from baseline indicates improvement in disease status.

See, e.g., Shiboski, Caroline H et al. “2016 American College of Rheumatology/European League Against Rheumatism Classification Criteria for Primary Sjagren's Syndrome: A Consensus and Data-Driven Methodology Involving Three International Patient Cohorts.” Arthritis & rheumatology (Hoboken, N.J.) vol. 69,1 (2017): 35-45.

In some embodiments, the treatment is effective to reduce SjD disease activity. In some embodiments, the disease activity is measured by a disease activity score selected from the group consisting of ESSDAI, the Sjogren's Syndrome Symptom Diary (SSSD), and ESSPRI. In some embodiments, the subject's score(s) may be measured before and after administration of the CD19-targeted cell therapy. In some embodiments, patient reported outcomes (PROs) are measured in the subject before and after administration of the CD19-targeted cell therapy.

In some embodiments, the treatment results in the subject achieving a change from baseline in ESSDAI score as compared to placebo. In some embodiments, the treatment results in the subject achieving≥3 points reduction from baseline in ESSDAI score. In some embodiments, the treatment results in the subject achieving a ESSDAI≤5. In some embodiments, the treatment results in the subject achieving meaningful improvement in the SSSD. In some embodiments, the treatment results in the subject achieving a change from baseline in stimulated whole salivary flow rate. In some embodiments, the treatment results in the subject achieving≥1 point or 15% reduction from baseline in ESSPRI.

In some embodiments, the treatment in accord with the provided methods results in clinical remission of SjD in the subject that is maintained for greater than 3 months. In some embodiments, the treatment in accord with the provided methods results in clinical remission of SjD in the subject that is maintained for greater than 4 months, 5 months, 6 months, 7 months, 8 months, 9 months, 10 months, 11 months, 12 months, 18 months, 24 months, 3 years, 4 years, 5 years or more. In some embodiments, the treatment in accord with the provided methods results in clinical remission of SjD in the subject that is maintained for greater than 6 months. In some embodiments, the treatment in accord with the provided methods results in clinical remission of SjD in the subject that is maintained for greater than 12 months. In some embodiments, the treatment in accord with the provided methods results in clinical remission of SjD in the subject that is maintained for greater than 24 months. In some embodiments, the treatment in accord with the provided methods results in clinical remission of SjD in the subject that is maintained for greater than 3 years. In some embodiments, the treatment in accord with the provided methods results in clinical remission of SjD in the subject that is maintained for greater than 4 years. In some embodiments, the treatment in accord with the provided methods results in clinical remission of SjD in the subject that is maintained for greater than 5 years.

In some embodiments, the treatment in accord with the provided methods results in prolonged remission. In some embodiments, prolonged remission is defined as a 5-year consecutive period of no disease activity and without treatment (e.g., corticosteroids, antimalarials, or immunosuppressants).

In some embodiments, the number of CAR+ T cells in a biological sample obtained from the patient, e.g., blood, may be determined at a period of time after administration of the cell therapy, e.g., to determine the pharmacokinetics of the cells. In some embodiments, number of CAR+ T cells, optionally CAR+ CD8+ T cells and/or CAR+ CD4+ T cells, detectable in the blood of the subject, or in a majority of subjects so treated by the method, is greater than 1 cells per μL, greater than 5 cells per μL or greater than 10 cells per μL.

### 3. ANCA-associated Vasculitis (AA V)

The anti-neutrophil cytoplasmic antibody (ANCA)-associated vasculitis (AAVs) are a group of disorders involving severe, systemic, small-vessel vasculitis and are characterized by the development of autoantibodies to the neutrophil proteins leukocyte proteinase 3 (PR3-ANCA) or myeloperoxidase (MPO-ANCA). AAVs cause inflammation to small and medium blood vessels. The three AAV subgroups, namely granulomatosis with polyangiitis (GPA), microscopic polyangiitis (MPA), and eosinophilic GPA (EGPA), are defined according to clinical features. The link between these clinical syndromes (GPA, EGPA, and MPA) and antineutrophilic cytoplasmic antibody (ANCA) was established in 1988 when the sera of patients having crescentic glomerulonephritis were found to bind to neutrophils in two recognizable patterns: perinuclear or diffusely throughout the cytoplasm. See, e.g., Qasim A, Patel J B. ANCA Positive Vasculitis. [Updated 2023 May 22]. In: StatPearls. Treasure Island (FL): StatPearls Publishing; 2024 Jan. and Kitching, A Richard et al. “ANCA-associated vasculitis.” Nature reviews. Disease primers vol. 6,1 71. 27 Aug. 2020.

In some embodiments, a biopsy from lung tissue shows evidence of vasculitis and necrotizing granuloma formation in subjects with GPA, however, a kidney biopsy does not show granulomas. In some embodiments, a biopsy shows capillaritis without granuloma formation in addition to leukocytoclastic changes and crescentic glomerulonephritis in kidneys in subjects with MPA. In some embodiments, a biopsy shows necrotizing vasculitis with eosinophilic infiltrates and eosinophilic granulomas in subjects with EGPA. In some embodiments, a blood test for ANCA may support a suspected diagnosis. In some embodiments, a urine test to evaluate blood and protein levels and assess kidney impact may be used. Imaging tests to help identify abnormalities may also be useful in AAV diagnosis.

In some embodiments, the systemic autoimmune disease is AAV, such as GPA, MPA, or EGPA. AAV is associated with various clinical syndromes, such as fatigue, fever, cough, hemoptysis, abdominal pain, blood in urine, weakness, numbness in hands and feet, and weight loss. Subjects with GPA typically present symptoms of the upper respiratory tract, which include bloody nasal discharge, nasal ulceration, sinusitis, and chronic otitis media and symptoms of the lower respiratory tract secondary to lung nodules and alveolar hemorrhage sometimes may be severe and fatal. Subjects with MPA typically present symptoms of acute renal failure. Subjects with EGPA typically present symptoms of eosinophilic granulomatous lesions involving the skin, cardiac and gastrointestinal tract, and involvement of the peripheral nervous system is also common.

Subjects with AAV may be treated with immunosuppressant, including cyclophosphamide, and azathioprine. In some embodiments, AAV treatment may be steroids, immunosuppressive drugs, disease-modifying antirheumatic drugs (DMARDs), complement inhibitors, and/or intravenous immunoglobulin. Rituximab therapy has also been evaluated in AAV subjects. In some embodiments, rituximab therapy is used for induction therapy, causing induction of remission. About 30-50% of patients are considered treatment refractory if they fail immunosuppressant/DMARD therapy, IVIG, and/or have a disease that remains a significant functional burden despite treatment. Further, a high risk of relapse remains for patients with PR3-ANCA and those who remain PR3-ANCA positive at the end of induction therapy. Relapses reflect further episodes of inflammation and contribute to irreversible tissue damage, end-stage kidney failure, treatment-related toxicity, chronic morbidity, increased mortality and high health-related costs. More-effective strategies to prevent relapse in AAV are needed. See, e.g., Smets, I, and M J Titulaer. “Antibody Therapies in Autoimmune Encephalitis.” Neurotherapeutics: the journal of the American Society for Experimental NeuroTherapeutics vol. 19,3 (2022): 823-831.

Morbidity varies significantly but may include a range of debilitating symptoms, and AAV may be fatal, especially in refractory patients. There is lack of treatments for refractory AAV patients, as there is high morbidity associated with this patient population. There are currently no curative treatments on the market. In some embodiments, the refractory patients have high BVAS and did not fully respond to the initial induction treatment. In some embodiments, the subject to be treated has high BVAS, refractory to three or more lines of treatment, and has severe organ involvement (e.g., renal and pulmonary).

Among provided methods are methods of treatment that involve administering engineered cells or compositions comprising engineered cells, such as engineered T cells to subjects with AAV, including GPA, MPA, and EGPA. Also provided are methods and uses of provided CD19-directed CAR engineered cells (e.g., T cells) and/or compositions thereof, including methods for the treatment of subjects having AAV, including severe AAV, that involves administration of the engineered cells and/or compositions thereof. In some embodiments, CD19-directed CAR T cells are used as a post-induction treatment for maintenance of remission for a variable time period to prevent relapse. In certain embodiments, the subject has been diagnosed with the systemic autoimmune disease AAV. In some embodiments, the subject is selected for or identified as having severe AAV, such as by the presence of certain features or clinical manifestations that indicate the presence of severe AAV. Exemplary selection criteria are further described herein. In some embodiments, the subjects with AAV relapse after treatment with corticosteroids, cyclophosphamide, and/or rituximab. In some embodiments, the subjects with relapsed AAV have a Proteinase 3 (PR3)-ANCA serotype. In some embodiments, the subjects with relapsed AAV have lung involvement and upper respiratory tract (ENT) disease. In some embodiments, the subjects with relapsed AAV have the persistence of PR3-ANCA positivity at the end of induction treatment. In some embodiments, the subjects with relapsed AAV have a history of prior relapse. See, e.g., Woerner, K., & Nachman, P. H. (2021). What Is the Best Maintenance Therapy for ANCA Vasculitis?. Clinical journal of the American Society of Nephrology: CJASN, 16(12), 1906-1908.

In some embodiments, the methods and use of provided CD19-directed CAR engineered cells (e.g., T cells) and/or compositions thereof, include methods for the treatment of subjects with AAV that have failed at least one or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In some embodiments, methods include selection of subjects with relapsed AAV. In some embodiments, methods include selection of subjects with symptoms indicative of AAV, including GPA, MPA, and EGPA.

In some embodiments, the subjects have severe AAV. In some embodiments, patients with severe AAV have a high Birmingham Vasculitis Activity Score (BVAS) (e.g., greater than 16). In some embodiments, patients with severe AAV have relapsed. In some embodiments, patients are classified as relapsed when they had more than 30 days between AAV diagnosis and ICU admission and were admitted to ICU with new vasculitis involvement. In some embodiments, patients are classified as relapsed when they have a new or worsening disease activity that requires a change in treatment. Clinically, AAV involves many organ systems including the lungs, kidneys, skin, and nervous system. In some embodiments, relapsed patients have severe organ involvement. See, e.g., Ozdemir, Ugur et al. “Value of prognostic scores in antineutrophil cytoplasmic antibody (ANCA) associated vasculitis patients in intensive care unit: a multicenter retrospective cohort study from Turkey.” Turkish journal of medical sciences vol. 50,5 1223-1230. 26 Aug. 2020, and King, Catherine et al. “Predicting relapse in anti-neutrophil cytoplasmic antibody-associated vasculitis: a Systematic review and meta-analysis.” Rheumatology advances in practice vol. 5,3 rkab018. 9 Mar. 2021.

In some embodiments, the subjects have EGPA, GPA, or MPA. In some embodiments, the subjects have GPA or MPA. In some embodiments, the subjects have high BVAS (e.g., severe disease) and have failed to achieve remission with induction or the second line of treatment. In some embodiments, the subjects have relapsed and failed to achieve remission with induction or the second line of treatment. In some embodiments, the subjects have severe organ involvement and have failed to achieve remission with induction or the second line of treatment. In some embodiments, the subjects have recovered from AAV after induction therapy but still require treatment. In some embodiments, the recovered subjects require treatment to remain in remission.

In some embodiments, the subjects to be treated has life or organ threatening AAV disease. In some embodiments, the subjects receive induction therapy. In some embodiments, the induction therapy consists of a therapy selected from the group of cyclophosphamide, rituximab, and avacopan. In some embodiments, during the maintenance phase, the subjects receive rituximab, methotrexate, mycophenolate mofetil, azathioprine, and/or leflunomide. In some embodiments, refractory subjects receive intravenous immunoglobulin (IVIG). In some embodiments, refractory subjects receive an administration of a composition comprising engineered T cells.

In some embodiments, the subjects to be treated have a non-life or organ threatening AAV disease. In some embodiments, the subjects receive induction therapy. In some embodiments, the induction therapy consists of methotrexate and/or mycophenolate mofetil. In some embodiments, during the maintenance phase, the subjects receive rituximab, methotrexate, mycophenolate mofetil, azathioprine, and/or leflunomide. In some embodiments, refractory subjects receive intravenous immunoglobulin (IVIG). In some embodiments, refractory subjects receive an administration of a composition comprising engineered T cells. In some embodiments, the composition comprising engineered T cells is a post-induction therapy.

In any of the embodiments herein, at or immediately prior to the time of the administration of the composition comprising engineered T cells, the subject has relapsed following remission after treatment with or become refractory to one or more prior therapies for AAV.

In any of the embodiments herein, at or immediately prior to the time of the administration of the composition comprising engineered T cells, the subject has relapsed following remission after treatment with, or become refractory to, one or more prior therapies for AAV. In any of the embodiments herein, at or immediately prior to the time of the administration of the composition comprising engineered T cells, the subject has relapsed following treatment with, or become refractory to, one or more prior therapies for AAV. In any of the embodiments herein, the one or more prior therapies for AAV does not comprise another dose of cells expressing the CAR. In any of the embodiments herein, the one or more prior therapies include two prior disease modifying therapies (DMT) with one of the prior therapies constituting an anti-CD20 antibody. In some embodiments, the subjects have received induction therapy (e.g., anti-CD20 antibody) and are post-induction. In some embodiments, the subjects are administered the composition comprising engineered T cells as a post-induction therapy. In any of the embodiments herein, the one or more prior therapies for AAV may comprise immunosuppressant, including cyclophosphamide, and azathioprine, and rituximab therapy. In any of the embodiments herein, CD19-directed CAR engineered cells (e.g., T cells) and/or compositions thereof are used to treat patients with AAV that are refractory to prior therapies.

a. Response and Efficacy

In some embodiments, the provided methods and uses involving administration of an anti-CD19 CAR T cell therapy reducing AAV disease activity in the subject.

In some embodiments, a subject who has been treated in accord with the provided methods is evaluated or monitored after treatment for a period of time to determine whether a complete or partial remission has occurred. In some embodiments, the subject is evaluated or monitored to assess whether the remission achieved according to the measurement is being maintained.

Birmingham Vasculitis Activity Score (BVAS) has been used for assessing the activity and severity of AAV, and it was reportedly associated with the poor prognosis of AAV. Five-factor score (FFS) was also used to predict the poor prognosis. In some embodiments, the treatment results in the subject having achieved remission, defined as a Birmingham Vasculitis Activity Score for Wegener's granulomatosis (BVAS/WG)≤1. In some embodiments, the treatment results in the subject having achieved remission, defined as a Birmingham Vasculitis Activity Score for Wegener's granulomatosis (BVAS/WG)≤1 and prednisone/prednisolone dose≤10 mg/day. In some embodiments, the treatment results in the subject having achieved remission, defined as a decrease or negative percent change from baseline in Vasculitis Damage Index (VDI). In some embodiments, the treatment results in the subject having improvement in quality of life as assessed by AAV patient-reported outcomes (PRO).

In some embodiments, the treatment in accord with the provided methods results in clinical remission of AAV in the subject that is maintained for greater than 3 months. In some embodiments, the treatment in accord with the provided methods results in clinical remission of AAV in the subject that is maintained for greater than 4 months, 5 months, 6 months, 7 months, 8 months, 9 months, 10 months, 11 months, 12 months, 18 months, 24 months, 3 years, 4 years, 5 years or more. In some embodiments, the treatment in accord with the provided methods results in clinical remission of AAV in the subject that is maintained for greater than 6 months. In some embodiments, the treatment in accord with the provided methods results in clinical remission of AAV in the subject that is maintained for greater than 12 months. In some embodiments, the treatment in accord with the provided methods results in clinical remission of AAV in the subject that is maintained for greater than 24 months. In some embodiments, the treatment in accord with the provided methods results in clinical remission of AAV in the subject that is maintained for greater than 3 years. In some embodiments, the treatment in accord with the provided methods results in clinical remission of AAV in the subject that is maintained for greater than 4 years. In some embodiments, the treatment in accord with the provided methods results in clinical remission of AAV in the subject that is maintained for greater than 5 years.

In some embodiments, the treatment in accord with the provided methods results in prolonged remission. In some embodiments, prolonged remission is defined as a 5-year consecutive period of no disease activity and without treatment (e.g., corticosteroids, antimalarials, or immunosuppressants).

See, e.g., Smith, Rona M et al. “Rituximab versus azathioprine for maintenance of remission for patients with ANCA-associated vasculitis and relapsing disease: an international randomised controlled trial.” Annals of the rheumatic diseases vol. 82,7 (2023): 937-944, Kim, Minyoung Kevin et al. “Multivariable index for assessing the activity and predicting all-cause mortality in antineutrophil cytoplasmic antibody-associated vasculitis.” Journal of clinical laboratory analysis vol. 34,1 (2020): e23022, and Robson, Joanna C et al. “Validation of the ANCA-associated vasculitis patient-reported outcomes (AAV-PRO) questionnaire.” Annals of the rheumatic diseases vol. 77, 8 (2018): 1157-1164.

### 4. Rheumatoid Arthritis (RA)

In some embodiments, the systemic autoimmune disease is Rheumatoid Arthritis (RA). RA is a chronic autoimmune inflammatory disease characterized by a destructive inflammation of the joints, which may lead to progressive disability and a reduced life expectancy. RA may result in joint damage, pain, stiffness, inflammation, and may also induce pulmonary and/or cardiac inflammation. RA is a chronic condition, however the severity and/or duration of symptoms may vary and people with RA experience intermittent bouts of intense disease activity, called flares. For some patients, the disease is continuously active and may worsen over time. With treatment, there may be periods of remission, where there is no disease activity or symptoms.

The synovial membrane in RA is infiltrated by activated immune cells, most abundantly macrophages and T cells, resulting in the chronic production of proinflammatory cytokines and matrix metalloproteinases, leading to inflammation and cartilage and bone degradation (Choy E H and Panayi G S, N Engl J Med. 2001; 344:907-916).

The American College of Rheumatology (ACR) proposed a set of criteria for classifying RA. The commonly used criteria are the ACR 1987 revised criteria (Arnett et al. Arthritis Rheum. 31:315-324 1988). Diagnosis of RA according to the ACR criteria requires a patient to satisfy a minimum number of listed criteria, such as tender or swollen joint counts, stiffness, pain, radiographic indications and measurement of serum rheumatoid factor. ACR 20, ACR 50 and ACR 70 are commonly used measures to express efficacy of RA therapy, particularly in clinical trials. ACR 20 represents a 20% improvement in the measured ACR criteria. Analogously, ACR 50 represents a 50% improvement in the measured ACR criteria, and ACR 70 represents a represents a 70% improvement in the measured ACR criteria. An individual, patient-reported measure of disability in RA patients is the Health Assessment Questionnaire Disability Index (HAQ-DI). HAQ-DI scores represent physical function in terms of the patient's reported ability to perform everyday tasks, including the level of difficulty they experience in carrying out the activity. By recording patients' ability to perform everyday activities, the HAQ-DI score may be used as one measure of their quality of life.

A patient to be treated may have RA as determined according to the 1987 ACR criteria. The patient may test positive for rheumatoid factor (RF) and/or anti-cyclic citrullinated peptide (CCP) IgG antibodies prior to treatment. RF positive and anti-CCP antibody positive statuses confirm diagnosis of RA. X-rays, ultrasounds, and MRIs may also diagnose the presence and severity of joint inflammation and damage. The patient may have had RA for a duration of at least 5 years or at least 7 years, for example between 5 and 10 years.

Serological status may also be used to classify RA patients. Patients may be rheumatoid factor (RF) positive, Anti-Citrullinated Protein Antibodies (ACPAs) positive, RF-positive and ACPA-positive, or seronegative. In some embodiments, RF-positive and/or ACPA-positive subjects are more likely to experience more severe disease progression.

RA is associated with joint pain, stiffness, swelling, and with loss of mobility in severe cases. Extra-articular involvement may lead to systemic symptoms such as fatigue, fever, and weight loss. Common extra-articular manifestations are rheumatoid nodules, firm, non-tender lumps that typically form over pressure points or areas prone to injury, such as the elbows and fingers. In some embodiments, extra-articular involvement comprises cardiac involvement, interstitial lung disease (ILD), and neuropathy. Extra-articular progressions are highly heterogeneous in quality-of-life impact (e.g., may impact skin, eye, heart, lung, renal, nervous, and gastrointestinal systems). Other disease associations include heart disease and diabetes from reduced mobility.

RA treatment is generally lifelong, and may include rest, strengthening exercises, pharmacologic treatment, and surgery. Pharmacologic agents used include disease modifying anti-rheumatic drugs (DMARDs, e.g. methotrexate), anti-inflammatories, anti-TNFs, corticosteroids, and other drugs.

DMARDs typically used in treating RA include methotrexate, hydroxychloroquine, sulfasalazine, and leflunomide. Anti-TNF-alpha inhibitors (e.g., IL6, CD80, CD86 inhibitors) include etanercept, infliximab, adalimumab, golimumab, and certolizumab pegol. Other biologic DMARDs include interleukin (IL) 6 inhibitors such as tocilizumab and sarilumab, T-cell costimulation inhibitors such as abatacept (CTLA4-Ig), and the anti-CD20 B-cell depleting monoclonal antibody such as rituximab. Targeted synthetic DMARDs include Janus kinases (JAK) inhibitors such as tofacitinib, baricitinib, and upadacitinib. See, e.g., Chauhan K, Jandu J S, Brent L H, et al. Rheumatoid Arthritis. [Updated 2023 May 25]. In: StatPearls. Treasure Island (FL): StatPearls Publishing; 2024 Jan.

Nonbiologic DMARDs may also be used. This category includes methotrexate, hydroxychloroquine (HCQ), azathioprine (AZA), sulfasalazine, leflunomide, and cyclosporine. Methotrexate is the initial drug of choice for patients with RA. NSAIDs do not have any disease-modifying effects but are commonly used to relieve symptoms related to joint inflammation and pain. Corticosteroids are also commonly used in patients with RA.

Many of the currently available treatments are associated with serious toxicity concerns, including infection due to immunosuppression and risk of malignancies. While these treatments are effective in some patients, physicians do not view their population-wide response rate as justifying their serious safety concerns. Additionally, while achieving remission is essential for preventing joint destruction and extra articular disease, it may be challenging to maintain. The primary treatment goal in RA is to treat aggressively in early lines to induce disease remission and prevent joint deterioration or progression to extra glandular disease However, responsive patients usually require treatment cycling every 3-5 years while unresponsive patients move quickly through high toxicity treatments with less than complete responses. Early treatment options are limited, decreasing likelihood of achieving remission with one line of treatment.

In some embodiments, the methods and use of provided CD19-directed CAR engineered cells (e.g., T cells) and/or compositions thereof, include methods for the treatment of subjects with RA that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory after four or more prior therapies for RA. In some embodiments, the subject is refractory to three or more prior therapies for RA, has high disease activity, and severe extra-articular disease. In some embodiments, the refractory subject comprises a high disease activity score (DAS28), RF-positive and ACPA-positive, and severe organ involvement (e.g., interstitial lung disease, peripheral nervous system disease, renal disease).

In some embodiments, methods include selection of subjects with relapsed RA. In some embodiments, methods include selection of subjects with symptoms indicative of RA.

In some embodiments, the subject has an insufficient response to two prior treatments. In some embodiments, the subject has an insufficient response to three prior treatments. In some embodiments, the subject has an insufficient response to four or more prior treatments. In some embodiments, the subject has failed to attain clinical remission (e.g., after three months of a given treatment) after having been treated with any two or more prior treatments for RA. In any embodiments, the subject is identified or selected as having an insufficient response to a prior treatment at a time prior to leukapheresis in connection with engineering the CD19-directed CAR T cell composition. Insufficient response to treatments is defined as a lack of response, insufficient response, or a lack of sustained response to appropriate doses. Intolerance is not considered an insufficient response.

In any of the embodiments herein, at or immediately prior to the time of the administration of the composition comprising engineered T cells, the subject has relapsed following remission after treatment with, or become refractory to, one or more prior therapies for RA. In any of the embodiments herein, at or immediately prior to the time of the administration of the composition comprising engineered T cells, the subject has relapsed following treatment with, or become refractory to, one or more prior therapies for the RA. In any of the embodiments herein, the one or more prior therapies for the RA does not comprise another dose of cells expressing the CAR. In any of the embodiments herein, the one or more prior therapies include two prior disease modifying therapies (DMT) with one of the prior therapies constituting an anti-CD20 antibody. In some embodiments, the subjects have received induction therapy (e.g., anti-CD20 antibody) and are post-induction. In some embodiments, the subjects are administered the composition comprising engineered T cells as a post-induction therapy.

In some embodiments, the subject to be treated is: a subject that is refractory to four or more prior therapies, which includes subjects who are among the most difficult to treat. In some embodiments, at least two of the previous treatments are with a biologic or targeted synthetic DMARDs. In some embodiments, the subject has a high disease activity, measured using Clinical Disease Activity Index (CDAI). In some embodiments, the subject has severe extra-articular disease (e.g., ILD, PNS/CNS, renal disease). In some embodiments, the subject to be treated is refractory to three or more prior therapies and has severe organ involvement. For example, the subject has failed multiple immunotherapies and has ILD or PNS/renal involvement. In some embodiments, the subject to be treated is refractory to three or more prior therapies, has high DAS28, and dual serological positivity. For example, the subject has failed three or more immunotherapies, has a high disease severity score, and is RF-positive and ACPA-positive. In some embodiments, the subject to be treated is refractory to six or more prior therapies. For example, the subject has failed six or more immunotherapies. In some embodiments, the provided methods and uses involving administration of an anti-CD19 CAR T cell therapy reduce RA disease activity in the subject. In some embodiments, a reduction in RA disease activity is evident where there is a clinical benefit to the subject after administration of anti-CD19 CAR T cell therapy.

See, e.g., Watanabe, Ryu et al. “Prevalence and predictive factors of difficult-to-treat rheumatoid arthritis: the KURAMA cohort.” Immunological medicine vol. 45,1 (2022): 35-44, Watanabe, Ryu et al. “Difficult-to-treat rheumatoid arthritis: Current concept and unsolved problems.” Frontiers in medicine vol. 9 1049875. 24 Oct. 2022, and Hunter, Theresa M et al. “Prevalence of rheumatoid arthritis in the United States adult population in healthcare claims databases, 2004-2014.” Rheumatology international vol. 37,9 (2017): 1551-1557. a. Response and Efficacy

One measure of how well RA is being controlled is the Disease Activity Score (DAS) (Fransen & van Riel Clin Exp Rheumatol 23:S93-S99 2005). The DAS is calculated by a medical practitioner based on various validated measures of disease activity, including physical symptoms of RA. A reduction in DAS reflects a reduction in disease severity. A DAS of less than 2.6 indicates disease remission. DAS between 2.6 and 3.2 indicates low disease activity. A DAS greater than 3.2 indicates increased disease activity and at this level a patient's therapy could be reviewed to determine whether a change in therapy is warranted. DAS greater than 5.1 indicates severe disease activity. Variations in calculating DAS may include assessing different numbers of joints in the patient and monitoring different blood components. DAS28 is the Disease Activity Score in which 28 joints in the body are assessed to determine the number of tender joints and the number of swollen joints (Prevoo et al. Arthritis Rheum 38:44-48 1995). When the DAS28 calculation includes a measurement of C-reactive protein (CRP) rather than erythrocyte sedimentation rate (ESR), it is referred to as DAS28-CRP (Smolen et al. Rheumatology 42:244-257 2003; Wells G, et al. Annals of the Rheumatic Diseases 68: 954-960 2009). CRP is believed to be a more direct measure of inflammation than ESR and is more sensitive to short term changes (Kushner, Arthritis Rheum 34: 1065-68 1991). CRP production is associated with radiological progression in RA (van Leeuwen M A, et al. Br J Rheumatol 32(suppl 3):9-13 1993) and is considered at least as valid as ESR to measure RA disease activity (Mallya R K, et al. J Rheumatol 9:224-8 1982; Wolfe F. J Rheumatol 24: 1477-85 1997).

The clinical benefit may comprise remission of RA. Typically, remission is defined by a DAS28-CRP of less than 2.6.

The clinical benefit may be an improvement of at least 20%, at least 50% or at least 70% treatment efficacy as determined by the 1987 ACR criteria, i.e. the clinical benefit may be achieving ACR 20, ACR 50 or ACR 70, respectively.

Other disease activity index such as Simple Disease Activity Index (SDAI) and Clinical Disease Activity Index (CDAI) for rheumatoid arthritis correlate well with DAS28. SDAI is the numerical sum of five outcome parameters: tender and swollen joint count (based on a 28-joint assessment), patient and physician global assessment of disease activity [visual analogue scale (VAS) 0-10 cm]and level of C-reactive protein (mg/dl, normal≤1 mg/dl). Remission is defined as an SDAI of<3.3, low disease activity as ≤11, moderate disease activity as ≤26 and high disease activity as ≥26. CDAI is calculated as follows: SDAI=SJC+ TJC+PGA+EGA, where: SJC=Swollen Joint Count, TJC=Tender Joint Count, PGA=Patient Global Assessment of Disease Activity, and EGA=Evaluator Global Assessment of Disease Activity. CDAI scores are grouped as follows: CDAI<=2.8: remission, CDAI≥2.8 and <=10: low disease activity, CDAI≥10 and <=22: moderate disease activity, and CDAI≥22: high disease activity. Patients may be classified into normal, low, moderate, and high RA based on DAS28.

A form of clinical benefit that is of particular value to RA patients is an improvement in their ability to perform everyday activities. Methods of the disclosure may comprise improvement in the patient's self-assessed disability measured by the Health Assessment Questionnaire, known as HAQ-DI. Methods comprising providing clinical benefit to an RA patient, wherein the clinical benefit comprises improving physical function of an RA patient as determined by HAQ-DI, and compositions and kits for use in such methods, are all aspects of the disclosure. Clinical benefit may comprise improving physical function of an RA patient as determined by HAQ-DI. In certain embodiments, a statistically significant improvement in HAQ-DI is achieved within twelve, ten, eight or six weeks of starting treatment according to the disclosure, or within four weeks, or within two weeks. The improvement may be at least a 0.25 improvement in HAQ-DI, i.e. a reduction of 0.25 or more in the patient's HAQ-DI score. In certain embodiments, the improvement is at least a 0.30, 0.40 or 0.45 improvement in HAQ-DI score. Improvement is generally measured with reference to the patient's baseline average HAQ-DI score prior to treatment with an inhibitor according to the disclosure.

Patients may be monitored during and/or following a course of treatment with anti-CD19 CAR T cell therapy, to assess the level of clinical benefit, for example by measuring DAS28-CRP and/or determining clinical benefit according to the ACR criteria and/or measuring HAQ-DI. The method may comprise determining that the clinical benefit is achieved, e.g. that the specified reduction in DAS28-CRP, and/or achievement of ACR 20, ACR 50 or ACR 70 is met, and/or that the HAQ-DI score is improved, as discussed elsewhere herein.

### 5. Autoimmune Encephalitis (AE)

Autoimmune encephalitis (AE) is an immune-mediated condition that induces brain inflammation and is one of the common causes of non-infectious encephalitis. In the past decade, AE has become an emerging addition to the differential diagnosis when a classical infection cannot explain focal neurological symptoms. While the exact mechanism of AE is unknown, current literature suggests that autoimmune antibodies target synaptic proteins, leading to widespread inflammation. AE is thought to be caused by autoantibodies that target key synaptic proteins, ion channels or receptors; the binding of antibodies results in decreased expression and suppression of receptor proteins. There are several types of AE, varying with antibody types; LGI1 is the second most frequently seen antibody in AE after NMDAR, which is observed more frequently in elderly males. Patients may cycle back and forth between acute and chronic phases of AE due to flares and post-acute maintenance shifts, and progression of the disease may be rapid.

AE commonly presents as a new onset of memory loss, psychosis, altered mental status, or seizures, with the presentation taking place over a few weeks to three months. AE involves several parts of the nervous system, including the limbic system, the spinal cord, and/or the entire neuroaxis. Specifically, AE may result in cognitive dysfunction (impaired memory, attention deficits), dyskinesia/dystonia, sleep disorders (insomnia, hypersomnolence), neuropsychiatric symptoms (mania, psychosis, agitation, catatonia), and/or compulsive behavior and altered sexual behavior.

AE may be confirmed by multiple modalities, including laboratory testing (antibody detection), neuroimaging, and electrophysiological studies (electroencephalogram).

To meet AE criteria, patients require subacute onset (rapid progression of less than 3 months) of working memory deficits (short-term memory loss), altered mental status (altered level of consciousness, lethargy, or personality change) or psychiatric symptoms with at least 1 of new focal CNS findings, new-onset seizures, CSF pleocytosis, and supportive MRI. These tests are complemented by AE-specific IgG antibody testing, CSF IgG index and oligoclonal bands, EEG, and brain biopsy to further refine diagnostic certainty. See, e.g., Orozco, Emma et al. “Autoimmune Encephalitis Criteria in Clinical Practice.” Neurology. Clinical practice vol. 13,3 (2023): e200151.

Treatment for AE is generally focused on treating acute crisis and exacerbations, but patients may receive long-term immunosuppression as well. First-line therapy for AE includes corticosteroids (1 g intravenous methylprednisolone for 3 to 5 days), intravenous immunoglobulins (0.4 g/kg/day for 5 days), and plasmapheresis alone or combined. Plasmapheresis should be considered in the treatment when patients have severe dysautonomia, refractory seizures, or central hypoventilation syndrome. Second-line immunotherapy should be considered for patients who fail to improve on these regimens. This includes rituximab (375 mg/m² for 4 weeks) and cyclophosphamide (750 mg/m² for 6 months) alone or combined in the adult population. See., e.g., Gole S, Anand A. Autoimmune Encephalitis. [Updated 2023 Jan 2]. In: StatPearls. Treasure Island (FL): StatPearls Publishing; 2024 Jan.

In some embodiments, subjects with AE may be treated with a high-dose of corticosteroids, for example, with intravenous methylprednisolone at a dose of 1 g per day for 3-7 days. Intravenous Ig (IVIg) at a dose of 2 g/kg over 2-5 days is a relatively easy-to-use and timely option for fast immunomodulation when corticosteroids are contraindicated or when the clinical picture is suggestive of or known to be related to antibody-mediated disease (e.g., probable or definite NMDAR-antibody encephalitis). Plasma exchange is another treatment option for acute immunomodulation when corticosteroids are contraindicated or ineffective. Both rituximab and cyclophosphamide have been used as second-line agents for AE. See, e.g., Abboud, Hesham et al. “Autoimmune encephalitis: proposed best practice recommendations for diagnosis and acute management.” Journal of neurology, neurosurgery, and psychiatry vol. 92,7 (2021): 757-768.

The majority of patients (˜75%) may achieve remission with the first line of therapies such as IVIG, PLEX, or corticosteroids; however, these patients will likely still require some form of long-term immunosuppression to decrease the risk of relapse. In these cases, the risk of long-term immunosuppressants is outweighed by the benefit of preventing potential relapses. Further, about −10% of AE patients are refractory to second line of treatments, with limited options for other lines of treatment. Refractory patients suffer from the accumulation of debilitating neurological symptoms, driven by the inflammatory neuropathy from uncontrolled or frequently relapsing AE.

In some embodiments, subjects are treated with immunotherapies to induce remission (i.e., induction therapies). In some embodiments, rituximab therapy is used for induction therapy, causing induction of remission. In some embodiments, patients respond to the initial induction treatment, but require a therapy to remain in remission (i.e., post-induction therapy). AE is an incurable condition with an unmet need for a post-induction treatment that may abate AE long-term, especially as AE may strongly impact QoL due to neurological complications, especially in refractory patients. See, e.g., Abboud, Hesham et al. “Autoimmune encephalitis: proposed best practice recommendations for diagnosis and acute management.” Journal of neurology, neurosurgery, and psychiatry vol. 92,7 (2021): 757-768 and Yang, Jiawei, and Xueyan Liu. “Immunotherapy for Refractory Autoimmune Encephalitis.” Frontiers in immunology vol. 12 790962. 16 Dec. 2021.

Among provided methods are methods of treatment that involve administering engineered cells or compositions comprising engineered cells, such as engineered T cells to subjects with AE, including relapsed AE. Also provided are methods and uses of provided CD19-directed CAR engineered cells (e.g., T cells) and/or compositions thereof, including methods for the treatment of subjects having AE, including relapsed AE, that involves administration of the engineered cells and/or compositions thereof. In some embodiments, CD19-directed CAR T cells are used as a post-induction treatment for maintenance of remission for a variable time period to prevent relapse. In certain embodiments, the subject has been diagnosed with the systemic autoimmune disease AE. In some embodiments, the subject is selected for or identified as having severe AE, such as by the presence of certain features or clinical manifestations that indicate the presence of severe AE. Exemplary selection criteria are further described herein. In some embodiments, the AE subjects relapse after treatment. In some embodiments, the subjects with relapsed AE have a history of prior relapse.

In some embodiments, the subjects to be treated have acute AE disease. In some embodiments, the subjects receive induction therapy. In some embodiments, the subjects receive corticosteroids, IVIG, and/or PLEX as a first line of treatment. In some embodiments, the subjects receive rituximab and/or cyclophosphamide as a second line of treatment. In some embodiments, subjects receive an administration of a composition comprising engineered T cells. In some embodiments, the composition comprising engineered T cells is a post-induction therapy.

In some embodiments, the subjects to be treated have chronic AE disease. In some embodiments, the subjects receive rituximab, corticosteroids, IVIG, mycophenolate mofetil, and/or azathioprine as a first line of treatment. In some embodiments, subjects receive an administration of a composition comprising engineered T cells. In some embodiments, the composition comprising engineered T cells is a post-induction therapy.

In some embodiments, the methods and use of provided CD19-directed CAR engineered cells (e.g., T cells) and/or compositions thereof, include methods for the treatment of subjects with AE that have failed at least one or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for AE. In some embodiments, methods include selection of subjects with symptoms indicative of relapsed AE.

In any of the embodiments herein, at or immediately prior to the time of the administration of the composition comprising engineered T cells, the subject has relapsed following remission after treatment with, or become refractory to, one or more prior therapies for AE. In any of the embodiments herein, at or immediately prior to the time of the administration of the composition comprising engineered T cells, the subject has relapsed following treatment with, or become refractory to, one or more prior therapies for AE. In any of the embodiments herein, the one or more prior therapies for AE does not comprise another dose of cells expressing the CAR. In any of the embodiments herein, the one or more prior therapies include two prior disease modifying therapies (DMT) with one of the prior therapies constituting an anti-CD20 antibody.

In some embodiments, subjects with AE including subjects with severe and refractory AE and/or with no response to current therapies are treated. Subjects positive for LGI1 may be treated. Subjects having received an induction therapy (e.g., rituximab) may also be treated as a post-induction therapy. Further, subjects with severe and/or relapsed AE treated with one or more prior lines of treatment (e.g., intravenous methylprednisolone (IVMP), intravenous immunoglobulin (IVIG), and plasma exchange (PLEX)) are also treated. In some embodiments, subjects with relapsed/refractory AE treated with three or more prior lines of treatment (e.g., tocilizumab (TCZ), resiniferatoxin (RTX)) are also treated.

In some embodiments, the subject to be treated is a subject that is refractory to one or more prior therapies, which includes subjects who are among the most difficult to treat. In some embodiments, at least one of the previous treatments is IVMP, IVIG, PLEX. In some embodiments, the subject is refractory to one or more prior therapies and has severe AE. For example, the subject has severe disease and has failed multiple immunotherapies via two independent relapses. In some embodiments, the subject has recovered or is in remission from induction therapy. For example, the subject has received and responded well to induction therapy, but still requires treatment. In some embodiments, the subject to be treated has AE.

In any of the embodiments herein, the one or more prior therapies for AE may comprise corticosteroids, intravenous immunoglobulins, plasmapheresis, cyclophosphamide, and rituximab therapy. In any of the embodiments herein, CD19-directed CAR engineered cells (e.g., T cells) and/or compositions thereof are used to treat patients with AE that are refractory to prior therapies.

a. Response and Efficacy

In some embodiments, the provided methods and uses involving administration of an anti-CD19 CAR T cell therapy reducing AE disease activity in the subject.

In some embodiments, a subject who has been treated in accord with the provided methods is evaluated or monitored after treatment for a period of time to determine whether a complete or partial remission has occurred. In some embodiments, the subject is evaluated or monitored to assess whether the remission achieved according to the measurement is being maintained.

In some embodiments, the treated subject has clinical and/or radiological improvement after completion of CAR T cell therapy. In some embodiments, the improvement is 2-4 weeks after completion of CAR T cell therapy. A modified Rankin scale (mRs) may also be used to examine a patient's response. In some embodiments, the modified Rankin Scale ranks: 0-no symptoms; 1-no significant disability, and able to carry out all usual activities, despite some symptoms; 2-slight disability; able to look after own affairs without assistance, but unable to carry out all previous activities; 3-moderate disability, and requires some help, but able to walk unassisted; 4—moderately severe disability and unable to attend to own bodily needs without assistance, and unable to walk unassisted; 5-severe disability, requires constant nursing care and attention, bedridden, incontinent; 6-dead. A score of 0-2 was considered a favorable outcome, whereas a score of 3-6 was graded as an unfavorable one. In some embodiments, reducing AE disease activity in the subject may include lowering the mRs score. In some embodiments, subjects treated with CAR T cells achieve an mRS score of 0-2 in a period of time following the CAR T cell treatment.

In some embodiments, the number of CAR+ T cells in a biological sample obtained from the patient, e.g., blood, may be determined at a period of time after administration of the cell therapy, e.g., to determine the pharmacokinetics of the cells. In some embodiments, number of CAR+ T cells, optionally CAR+ CD8+ T cells and/or CAR+ CD4+ T cells, detectable in the blood of the subject, or in a majority of subjects so treated by the method, is greater than 1 cells per μL, greater than 5 cells per μL or greater than per 10 cells per μL.

### 6. Pemphigus

Pemphigus vulgaris (PV) (i.e., pemphigus foliaceus, pemphigus disease) is a rare autoimmune disease that causes blistering on cutaneous and mucosal surfaces. Patients may present with painful ulcerations of especially the buccal or palatine mucosa, but it may also present in the nose, genitals, anus, esophagus, and conjunctiva (Kavala et al. 2015; Kavala et al. 2011). In the skin, the bullae tend to rupture, because the cellular interconnections are weakened by the autoimmune attack on desmogleins 1 and 3 (Stanley and Amagai 2006). PV is caused by autoantibodies that target cadherins, specifically desmogleins (Dsg 1 and Dsg 3), though there may be some role for desmocollin; thus, this is a type 2 hypersensitivity reaction. Acantholysis, or the loss of keratinocyte-keratinocyte adhesion, is interrupted by circulating IgG autoantibodies to intercellular adhesion molecules. Many animal models have shown that enzymatic inactivation of Dsg 1 and gene deletion of Dsg 3 results in pathology similar to PV. See, e.g., Ingold C J, Sathe N C, Khan MAB. Pemphigus Vulgaris. [Updated 2024 Mar 1]. In: StatPearls. Treasure Island (FL): StatPearls Publishing; 2024 Jan.

PV is a blistering disease that initially presents on the oral mucosa in 80% of cases. These intraoral blisters often rupture, leaving painful erosions, which may affect patients' oral intake, leading to potential malnutrition. Mucosal P V may be found in the conjunctiva, nasal mucosa, larynx, pharynx, esophagus, penis, vagina, and anus. Severe mucosal and cutaneous involvement in PV subjects may require hospitalization.

Current first-line treatment includes systemic corticosteroids and anti-CD20 monoclonal antibodies. Anti-CD20 monoclonal antibodies, such as rituximab and ofatumumab, are a desirable modality to use in conjunction with corticosteroids for first-line treatment in moderate-to-severe pemphigus. Second-line treatment in combination with corticosteroids may include steroid-sparing immunosuppressives, such as azathioprine or mycophenolate mofetil (MMF). Third-line treatments for PV include intravenous immunoglobulin (IVIG), cyclophosphamide, dapsone, immunoadsorption, and methotrexate. Obinutuzumab, ofatumumab, and veltuzumab are anti-CD20 monoclonal antibodies that may be an alternative to rituximab. Efgartigimod, an engineered Fc fragment that inhibits the activity of the neonatal Fc receptor thereby reducing serum IgG levels, has also been tested in subjects with pemphigus vulgaris and pemphigus foliaceus, however failed to meet the goal endpoints in the clinical trial. See, e.g., Porro, Adriana Maria et al. “Pemphigus vulgaris.” Anais brasileiros de dermatologia vol. 94,3 264-278. 29 Jul. 2019.

In some embodiments, the methods and use of provided CD19-directed CAR engineered cells (e.g., T cells) and/or compositions thereof, include methods for the treatment of subjects with PV that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In some embodiments, methods include selection of subjects with symptoms indicative of PV. In some embodiments, methods include selection of subjects with symptoms indicative of relapsed PV.

In any of the embodiments herein, at or immediately prior to the time of the administration of the composition comprising engineered T cells, the subject has relapsed following remission after treatment with, or become refractory to one or more prior therapies for PV.

In any of the embodiments herein, at or immediately prior to the time of the administration of the composition comprising engineered T cells, the subject has relapsed following remission after treatment with, or become refractory to, one or more prior therapies for PV. In any of the embodiments herein, at or immediately prior to the time of the administration of the composition comprising engineered T cells, the subject has relapsed following treatment with, or become refractory to, one or more prior therapies for the PV. In any of the embodiments herein, the one or more prior therapies for the PV does not comprise another dose of cells expressing the CAR. In any of the embodiments herein, the one or more prior therapies include 2 prior disease modifying therapies (DMT) with one of the prior therapies constituting an anti-CD20 antibody. In some embodiments, the subject to be treated has relapsed and/or refractory disease to one or more prior therapies. In some embodiments, the subject has relapsed to or is refractory to Efgartigimod.

a. Response and Efficacy

In some embodiments, the treatment is effective to reduce PV disease activity. In some embodiments, the PV disease activity is measured by a disease activity score selected from the pemphigus disease area index (PDAI), Pemphigus Vulgaris Activity Score (PVAS), and autoimmune bullous skin disorder intensity score (ABSIS), which are independent disease severity assessments of pemphigus disease extent. The PDAI questionnaire has two components including activity and damage. The activity component consists of skin, scalp and mucosa parts, and the damage component consists of skin and scalp parts. The total activity score was used for the summary of PDAI scores. PDAI total activity score=Total skin activity+ Total scalp activity+Total mucosa activity. PDAI Total Activity Score ranged from 0 to 250 points representing disease activity (higher scores mean a worse outcome). Negative change in total activity score from baseline indicates improvement in pemphigus activity. The ABSIS, a scoring system with a maximum score of 206, uses the rule of 9s, which is used in burns measurement, to assess the percentage of involvement of blisters and erosions on the skin combined with a weighting factor for the stage of the blistering and erosions. See, e.g., Rahbar, Ziba et al. “Pemphigus disease activity measurements: pemphigus disease area index, autoimmune bullous skin disorder intensity score, and pemphigus vulgaris activity score.” JAMA dermatology vol. 150,3 (2014): 266-72 and H6bert, Vivien et al. “Large International Validation of ABSIS and PDAI Pemphigus Severity Scores.” The Journal of investigative dermatology vol. 139,1 (2019): 31-37, and Daniel, Benjamin S et al. “Severity score indexes for blistering diseases.” Clinics in dermatology vol. 30,1 (2012): 108-13.

In some embodiments, the subject's PDAI indices score may be measured before and after administration of the CD19-targeted cell therapy. In some embodiments, the subject's PSS indices score may be measured before and after administration of the CD19-targeted cell therapy. In some embodiments, the subject's ABSIS may be measured before and after administration of the CD19-targeted cell therapy. In some embodiments, the treatment results in a decrease of the PDAI score of the subject. In some embodiments, the treatment results in a decrease of the ABSIS score of the subject.

### 7. Membranous Nephropathy (MN)

Membranous nephropathy (MN), also known as membranous glomerulopathy, is one of the many glomerular diseases causing nephrotic syndrome. It is characterized by massive proteinuria (≥3.5 g/day) and clinically presents with peripheral edema, hypertension, frothy urine, and manifestations of thromboembolic phenomena. Membranous nephropathy is classified into Primary and Secondary. About 80% of cases are renal limited (primary MN, PMN) and 20% are associated with other systemic diseases or exposures (secondary MN). Primary membranous nephropathy (PMN) is a kidney-specific autoimmune disease that is caused by circulating antibodies against certain native podocyte antigens, specifically phospholipase A2 receptor (PLA2R) and thrombospondin type-1 domain-containing 7A (THSD7A). The PLA2R antibody concentration also has prognostic implications. Low antibody concentrations are associated with a higher likelihood of spontaneous remission, whereas higher concentrations are associated with development of nephrotic syndrome (if not initially present) and loss of kidney function. Common causes of secondary MN including hepatitis B and C, lupus, and sarcoid. See, e.g., Couser, William G. “Primary Membranous Nephropathy.” Clinical journal of the American Society of Nephrology: CJASN vol. 12,6 (2017): 983-997 and Alok A, Yadav A. Membranous Nephropathy. [Updated 2023 Jun 5]. In: StatPearls. Treasure Island (FL): StatPearls Publishing; 2024 Jan.

MN is caused by the deposition of antigen-antibody complexes between the glomerular basement membrane (GBM) and podocytes. These complexes mainly consist of immunoglobulin IgG4, complement C3, and C5b-C9 membrane attack complexes (MAC). The immune complexes activate the complement system and generate membrane attack complex (MAC), which releases proteases, cytokines, and oxidants, causing cellular and tissue damage. This leads to disruption of podocyte structure, hampering of slit diaphragm integrity, and loss of membrane anionic charge barrier, resulting in proteinuria. This nephrotic range proteinuria leads to hyperlipidemia, prothrombotic state, vitamin D deficiency, and hypertension.

Current treatment includes symptomatic management with diuretics, statins, angiotensin-converting enzyme inhibitors (ACEi) or angiotensin receptor blockers (ARBs), systemic anticoagulant therapy (newer direct oral anticoagulant agents or vitamin K antagonist therapy), antihypertensives, and dietary salt restriction. Immunosuppressive therapy includes treatment with methylprednisolone, prednisone, cyclophosphamide, and in some cases with appropriate pneumocystis pneumonia (PCP) and antiviral prophylaxis (trimethoprim-sulfamethoxazole and valganciclovir). Rituximab may be used as a second-line therapy. Other drug options are chlorambucil, mycophenolate mofetil, and adrenocorticotropic hormone (ACTH) analogs.

Partial remission is defined as ≥50% reduction of proteinuria from baseline or between 0.3 and 3.5 g/d, with relatively stable eGFR. Relapse is defined as a recurrence of ≥3.5 g/d of proteinuria after remission. Certain risk factors are associate with poor prognosis, which include male gender, white race, old age, hypertension on presentation, massive proteinuria (≥8 g/day) for 6 months, elevated creatinine or acute kidney injury at the time of presentation, and extensive tubulointerstitial fibrosis on biopsy.

In some embodiments, the methods and use of provided CD19-directed CAR engineered cells (e.g., T cells) and/or compositions thereof, include methods for the treatment of subjects with MN that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject relapsed to or is refractory to one or more prior therapies for treating MN.

In any of the embodiments herein, at or immediately prior to the time of the administration of the composition comprising engineered T cells, the subject has relapsed following remission after treatment with, or become refractory to one or more prior therapies for MN.

In any of the embodiments herein, at or immediately prior to the time of the administration of the composition comprising engineered T cells, the subject has relapsed following remission after treatment with, or become refractory to, one or more prior therapies for MN. In any of the embodiments herein, at or immediately prior to the time of the administration of the composition comprising engineered T cells, the subject has relapsed following treatment with, or become refractory to, one or more prior therapies for the MN. In any of the embodiments herein, the one or more prior therapies for the MN does not comprise another dose of cells expressing the CAR. In any of the embodiments herein, the one or more prior therapies include 2 prior disease modifying therapies (DMT) with one of the prior therapies constituting an anti-CD20 antibody. a. Response and Efficacy

In some embodiments, the provided methods and uses involving administration of an anti-CD19 CAR T cell therapy reduce MN disease activity in the subject. In some embodiments, the reduced disease activity results in complete remission. In some embodiments, the reduced disease activity results in proteinuria≤0.3 g/d or 300 mg/g on spot urine protein creatinine ratio (UPCR). In some embodiments, the anti-CD19 CAR T cell therapy effectively reduces MN disease activity. In some embodiments, the MN disease activity is measured by a decrease in proteinuria, hypertension, and/or GFR levels. In some embodiments, the MN disease activity is measured by a decrease in PLA2R antibody concentrations. In some embodiments, the provided methods and uses involving administration of an anti-CD19 CAR T cell therapy result in prolonged survival time.

8. IgG4-related disease (IgG4-RD)

Immunoglobulin G4-related disease (IgG4-RD) is also known as IgG4-related systemic disease, hyper-IgG4 disease, IgG4-related autoimmune disease, IgG4-associated disease, IgG4-related sclerosing disease, and IgG4-syndrome. It is a multi-organ, fibro-inflammatory condition with tumefactive lesions of unknown etiology and characteristic histopathological features. The commonly involved organs are the pancreas, kidneys, orbital adnexal structures, salivary glands, and retroperitoneum. The pathological hallmark of this disease is dense lymphoplasmacytic infiltrate with IgG4 positive plasma cells, storiform fibrosis, obliterative phlebitis, and a variable amount of eosinophils. Elevated levels of serum IgG4 is frequent. See, e.g., Nambiar S, Oliver T I. IgG4-Related Disease. [Updated 2023 Aug 8]. In: StatPearls. Treasure Island (FL): StatPearls Publishing; 2024 Jan.

Diagnosis is based on clinical and histological features. There are two types of criteria available: (1) the Mayo Clinic HISORt criteria for the diagnosis of AIP and (2) the Japanese Comprehensive Clinical Diagnostic (CCD) criteria for IgG4-RD. Histopathology is the current “gold standard” for diagnosis. Elevated serum IgG 4 levels of more than 1.4 g/L is seen in 70% to 80% of patients. IgG4 elevation more than two times the upper limit of normal (normal 140 mg/dL) has 99% specificity for IgG4-RD.

Patients with IgG4-RD may be treated with corticosteroids and prednisone. In patients with the recurrent or refractory disease who are on steroid taper or discontinuation, second-line agents that may be tried include azathioprine, mycophenolate mofetil, and methotrexate. B-cell depletion using rituximab is another mode of treatment.

In some embodiments, methods include selection of subjects with symptoms indicative of IgG4-RD. In some embodiments, methods include selection of subjects with symptoms indicative of relapsed IgG4-RD.

In some embodiments, the methods and use of provided CD19-directed CAR engineered cells (e.g., T cells) and/or compositions thereof, include methods for the treatment of subjects with IgG4-RD that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for IgG4-RD.

In any of the embodiments herein, at or immediately prior to the time of the administration of the composition comprising engineered T cells, the subject has relapsed following remission after treatment with, or become refractory to, one or more prior therapies for IgG4-RD. In any of the embodiments herein, at or immediately prior to the time of the administration of the composition comprising engineered T cells, the subject has relapsed following treatment with, or become refractory to, one or more prior therapies for the IgG4-RD. In any of the embodiments herein, the one or more prior therapies for the IgG4-RD does not comprise another dose of cells expressing the CAR. In some embodiment, the prior therapy is an anti-CD20 antibody, such as rituximab. In some embodiments, the subject is refractory to treatment with an anti-CD20 antibody, such as is a rituximab-refractory patient. In any of the embodiments herein, the one or more prior therapies include 2 prior disease modifying therapies (DMT) with one of the prior therapies constituting an anti-CD20 antibody.

a. Response and Efficacy

In some embodiments, the provided methods and uses involving administration of an anti-CD19 CAR T cell therapy reduce IgG4-RD disease activity in the subject. In some embodiments, the reduced disease activity results in complete remission. In some embodiments, the reduced disease activity results in a decrease in IgG4 serum levels. The IgG4-RD responder index (IgG4-RD RI) may be used as an outcome measure in subjects treated for IgG4-RD. The IgG4-RD RI uses a scoring system from 0-4 for each organ system or site and asks the clinician to rate the extent of disease activity and damage at the time of the clinical encounter. The scores refer to manifestations of disease activity; 0=normal or resolved, 1=improved, 2=persistent (unchanged from previous visit; still active), 3=new/recurrence, and 4=worsened despite treatment. In some embodiments, the reduced disease activity results in a decrease in IgG4-RD score. In some embodiments, the reduced disease activity results in scores of 0-1. In some embodiments, the reduced disease activity results in a negative percent change in Immunoglobulin G4-Related Disease Responder Index (IgG4-RD RI) Score.

See, e.g., Carruthers, Mollie N et al. “Development of an IgG4-RD Responder Index.” International journal of rheumatology vol. 2012 (2012): 259408.

### 9. Neuromyelitis Optica Spectrum Disorder (NMOSD)

Neuromyelitis optica spectrum disorder (NMOSD; previously known as Devic disease or neuromyelitis optica [NMO]) is an inflammatory disorder of the central nervous system characterized by severe, immune-mediated demyelination and axonal damage predominantly targeting optic nerves and the spinal cord. This disorder is intricately linked to aquaporin-4 immunoglobulin G antibodies (AQP4-IgG), necessitating serologic testing for accurate evaluations. Aquaporin-4 is a transmembrane water channel found on the foot processes of astrocytes and is highly concentrated in certain parts of the central nervous system, such as the optic nerve, the spinal cord, and the area postrema. NMOSD is an autoimmune demyelinating astrocytopathy where the AQP4-IgG mediates perivascular lymphocytic infiltration, leading to axonal loss. See, e.g., Shumway C L, Patel B C, Tripathy K, et al. Neuromyelitis Optica Spectrum Disorder (NMOSD) [Updated 2024 Jan 8]. In: StatPearls. Treasure Island (FL): StatPearls Publishing; 2024 Jan.

NMOSD may cause optic neuritis, which presents with acute onset, painful, monocular vision loss, with a relative afferent pupillary defect in the affected eye. NMOSD has been increasingly recognized as a non-monophasic illness; rather, it follows a relapsing condition with events of disease exacerbation separated by years or decades. Concurrent features of SLE, myasthenia gravis, autoimmune thyroid disease, Sjagren syndrome, and antiphospholipid antibody syndrome may be present. The core clinical characteristics of NMOSD include optic neuritis, acute myelitis, area postrema syndrome (unexplained hiccups, nausea, or vomiting), acute brainstem syndrome, symptomatic narcolepsy or acute diencephalic clinical syndrome with NMOSD-typical diencephalic MRI lesions, and symptomatic cerebral syndrome with NMOSD-typical brain lesions. In some embodiments, the NMOSD subject has AQP4 autoantibodies.

Immunosuppressive therapy (e.g., corticosteroid) may be used to treat NMOSD. Treatment of the acute phase of NMOSD flare usually involves intravenous steroids such as methylprednisolone, often used in high doses (500-100 mg daily) for 5 to 10 days. Other studies advocate plasmapheresis treatment (55 mL/kg) and intravenous immunoglobulin. Chronic immunosuppressive therapy may be accomplished with azathioprine or rituximab as first-line agents. Second-line agents may include mycophenolate or methotrexate. Newer biologics are directed against specific immune mediators such as anti-IL-6, anti-complement, or anti-AQP4-IgG. Current recommendations include indefinite use of immunosuppressive therapy to prevent future attacks in all patients with aquaporin-4 antibodies. See, e.g., Huda, Saif et al. “Neuromyelitis optica spectrum disorders.” Clinical medicine (London, England) vol. 19,2 (2019): 169-176.

In some embodiments, the methods and use of provided CD19-directed CAR engineered cells (e.g., T cells) and/or compositions thereof, include methods for the treatment of subjects with NMOSD that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for NMOSD.

In any of the embodiments herein, the subject relapsed to or is refractory to one or more prior therapies for treating NMOSD. In any of the embodiments herein, at or immediately prior to the time of the administration of the composition comprising engineered T cells, the subject has relapsed following remission after treatment with, or become refractory to, one or more prior therapies for NMOSD. In any of the embodiments herein, at or immediately prior to the time of the administration of the composition comprising engineered T cells, the subject has relapsed following treatment with, or become refractory to, one or more prior therapies for the NMOSD. In any of the embodiments herein, the one or more prior therapies for the NMOSD does not comprise another dose of cells expressing the CAR. In any of the embodiments herein, the one or more prior therapies include 2 prior disease modifying therapies (DMT) with one of the prior therapies constituting an anti-CD20 antibody.

In some embodiments, methods include selection of subjects with symptoms indicative of NMOSD. In some embodiments, methods include selection of subjects with symptoms indicative of relapsed NMOSD. In some embodiments, methods include selection of subjects with AQP4-IgG levels above a threshold.

a. Response and Efficacy

In some embodiments, the provided methods and uses involving administration of an anti-CD19 CAR T cell therapy reduce NMOSD disease activity in the subject. In some embodiments, the reduced disease activity results in complete remission. In some embodiments, the reduced disease activity is seen as decrease in likelihood of relapse. In some embodiments, AQP4-IgG levels decrease as the disease activity is reduced in the subject. In some embodiments, the reduced disease activity results in AQP4-IgG seronegative serum. The Expanded Disability Status Scale (EDSS) and its associated functional system (FS) score provide a system for quantifying disability and monitoring changes in the level of disability over time. The EDSS score ranges from 0 (normal neurological exam) to 10 (death from MS). In some embodiments, the reduced disease activity results in decrease of EDSS score. In some embodiments, the reduced disease activity results in EDSS score of less than 3. In some embodiments, the reduced disease activity results in EDSS score of less than 2.

### 10. Stiff-Person Syndrome (SPS)

Stiff person syndrome (SPS) is a rare progressive and often underdiagnosed immune-mediated disorder of the central nervous system characterized by progressive rigidity and triggered painful spasms of predominantly axial and proximal limb muscles.

Currently, there are three clinical classifications of SPS: classic SPS, partial SPS variants, and progressive encephalomyelitis with rigidity and myoclonus (PERM). Classic SPS is a common clinical form, present in 70 to 80% of SPS patients. It is associated with anti-glutamic acid decarboxylase (anti-GAD) antibodies and with rigidity and stiffness of the trunk muscles, specifically in the thoracolumbar region. Several clinical variants of SPS have been described and include stiff limb syndrome, jerky SPS, cerebellar variant, SPS with epilepsy, and dystonia. The paraneoplastic variant is associated with breast, colon, thyroid, lung malignancies, Hodgkin and non-Hodgkin lymphomas and tends to clinically manifest before cancer itself.

B-cell-mediated autoimmune inflammation affects different components of inhibitory GABAergic neurons and their synapses. SPS is associated with high titers of autoantibodies to various components of inhibitory synapses. Production of autoantibodies against antigens involved in GABA synthesis and release within the central nervous system results in a dysfunction of major inhibitory pathways leading to impaired truncal and axial muscles impaired relaxation due to hyperexcitability the motor cortex.

Glutamic acid decarboxylase (GAD) is an intracellular enzyme that transforms glutamate into GABA and is a primary target and a common antigen identified in classic SPS. GAD exists in 2 isoforms: GAD67 and GAD65. The production of anti-GAD65 antibodies is a hallmark of a pathological process in classic SPS and is found in 70-80% of cases.

Current treatments are GABAergic (gamma-aminobutyric acid) therapy and immunotherapy. Benzodiazepines may be used as a first-line treatment, with the addition of levetiracetam or pregabalin if symptoms persist. As second-line therapy, oral baclofen, rituximab, and tacrolimus may be recommended. For patients with refractory treatment, intrathecal baclofen, intravenous immunoglobulin (IVIG), or plasmapheresis may be used.

See, e.g., Muranova A, Shanina E. Stiff Person Syndrome. [Updated 2023 Jul 10]. In: StatPearls. Treasure Island (FL): StatPearls Publishing; 2024 Jan., McKeon, Andrew et al. “Stiff-man syndrome and variants: clinical course, treatments, and outcomes.” Archives of neurology vol. 69,2 (2012): 230-8, Ortiz, Juan Fernando et al. “Stiff-Person Syndrome: A Treatment Update and New Directions.” Cureus vol. 12,12 el 1995. 9 Dec. 2020, and Alexopoulos, Harry, and Marinos C Dalakas. “Immunology of stiff person syndrome and other GAD-associated neurological disorders.” Expert review of clinical immunology vol. 9,11 (2013): 1043-53.

In some embodiments, the methods and use of provided CD19-directed CAR engineered cells (e.g., T cells) and/or compositions thereof, include methods for the treatment of subjects with SPS that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for SPS.

In some embodiments, methods include selection of subjects with symptoms indicative of SPS. In some embodiments, methods include selection of subjects with symptoms indicative of relapsed SPS.

In any of the embodiments herein, at or immediately prior to the time of the administration of the composition comprising engineered T cells, the subject has relapsed following remission after treatment with, or become refractory to, one or more prior therapies for SPS. In any of the embodiments herein, at or immediately prior to the time of the administration of the composition comprising engineered T cells, the subject has relapsed following treatment with, or become refractory to, one or more prior therapies for the SPS. In any of the embodiments herein, the one or more prior therapies for the SPS does not comprise another dose of cells expressing the CAR. In any of the embodiments herein, the one or more prior therapies include 2 prior disease modifying therapies (DMT) with one of the prior therapies constituting an anti-CD20 antibody.

a. Response and Efficacy

The effect or efficacy of treatment may be evaluated with various outcome measures, including Distribution of Stiffness Index (DSI) and heightened sensitivity score (HSS). DSI is a validated indicator of stiffness, and scores range from 0 to 6 and reflect the extent of stiffness. One point is given for stiffness in each of the following areas: lower trunk, upper trunk, legs, arms, face and abdomen. HSS measures changes in the frequency of spasms, and scores range from 1 to 7; one point given for each source or type of spasm: unexpected noises, visual stimuli, somatosensory stimuli, voluntary activities, emotional upset or stress, no specific stimuli, or nocturnal spasms.

In some embodiments, the provided methods and uses involving administration of an anti-CD19 CAR T cell therapy reduce SPS disease activity in the subject. In some embodiments, the reduced disease activity results in complete remission. In some embodiments, the reduced disease activity results in a reduction of DSI and HSS scores. In some embodiments, the reduced disease activity results in patients with >=2 points reduction in the distribution of stiffness index (DSI) and >=1 point reduction in heightened sensitivity score (HSS). In some embodiments, a patient is considered a responder having >=2 points reduction in the distribution of stiffness index (DSI) AND>=1 point reduction in heightened sensitivity score (HSS).

### 11. Irritable Bowel Disease (IBD)

Irritable bowel syndrome (IBS) or irritable bowel disease (IBD) is a chronic disorder of the gastrointestinal tract, characterized by abdominal pain and alterations in bowel habits. IBS is recognized as a multifactorial disorder, with the following among the proposed mechanisms contributing to symptomatology: gastrointestinal dysmotility, inflammation, visceral hypersensitivity, and altered intestinal microbiota. IBS has been categorized as a functional bowel disorder, defined by symptom onset greater than six months and recurrence at least three days per month during the last three months. Diagnosis is made according to a symptom-based classification system, the Rome Criteria, which includes subtypes of IBS patients based on their predominant stool pattern: constipation (IBS-C), diarrhea (IBS-D), mixed (IBS-M), or unsubtyped (IBS-U).

See, e.g., Patel N, Shackelford K B. Irritable Bowel Syndrome. [Updated 2022 Oct 30]. In: StatPearls. Treasure Island (FL): StatPearls Publishing; 2024 Jan. and Weaver, Kristen Ronn et al. “Irritable Bowel Syndrome.” The American journal of nursing vol. 117,6 (2017): 48-55.

Therapeutic options for relieving abdominal pain in IBS patients include antispasmodics, peppermint oil, selective serotonin reuptake inhibitors (SSRIs), and tricyclic antidepressants; interventions for bowel disturbances (IBS-D) include opioid agonists, antibiotics, bile salt sequestrants, probiotics, mixed opioid agonists/antagonists, and 5-HT3 antagonists, IBS-C is treated with chloride channel activators, polyethylene glycol (PEG), psyllium, and guanylate cyclase C agonists. Prosecretory agents, linaclotide and lubiprostone, are also recommended for the treatment of IBS-C.

In some embodiments, methods include selection of subjects with symptoms indicative of IBD. In some embodiments, methods include selection of subjects with symptoms indicative of relapsed IBD.

In some embodiments, the methods and use of provided CD19-directed CAR engineered cells (e.g., T cells) and/or compositions thereof, include methods for the treatment of subjects with IBD that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for IBD.

In any of the embodiments herein, the subject relapsed to or is refractory to one or more prior therapies for treating irritable bowel disease (IBD). In any of the embodiments herein, at or immediately prior to the time of the administration of the composition comprising engineered T cells, the subject has relapsed following remission after treatment with, or become refractory to, one or more prior therapies for IBD. In any of the embodiments herein, at or immediately prior to the time of the administration of the composition comprising engineered T cells, the subject has relapsed following treatment with, or become refractory to, one or more prior therapies for the IBD. In any of the embodiments herein, the one or more prior therapies for the IBD does not comprise another dose of cells expressing the CAR. In some embodiments, the subject is refractory to or has relapsed from treatment with an anti-CD20 antibody therapy, such as is refractory to or has relapsed following treatment with rituximab. In any of the embodiments herein, the one or more prior therapies include 2 prior disease modifying therapies (DMT) with one of the prior therapies constituting an anti-CD20 antibody.

a. Response and Efficacy

In some embodiments, the provided methods and uses involving administration of an anti-CD19 CAR T cell therapy reduce IBD disease activity in the subject. In some embodiments, the reduced disease activity results in complete remission. In some embodiments, the reduced disease activity results in an improvement in chronic abdominal symptoms. In some embodiments, the reduced disease activity results in less abdominal pain, less diarrhea, and/or less constipation.

### 12. Thrombotic Thrombocytopenia Purpura (TTP)

Thrombotic thrombocytopenic purpura (TTP) is a microangiopathic hemolytic anemia classically characterized by the pentad of fever, hemolytic anemia, thrombocytopenia, and renal and neurologic dysfunction. TTP results from either a congenital or acquired absence/decrease of the von Willebrand factor-cleaving protease ADAMTS13 (a disintegrin and metalloproteinase with a thrombospondin type 1 motif member 13). Low levels of ADAMTS13 activity result in microthrombi formation, which leads to end-organ ischemia and damage. This is due to the inability of the ADAMTS13 to inactivate the large multimer von Willebrand factor (VWF) that is necessary to prevent spontaneous coagulation. The central nervous system and kidneys are the two common organ systems affected by TTP. See, e.g., Stanley M, Killeen R B, Michalski J M. Thrombotic Thrombocytopenic Purpura. [Updated 2023 Apr 7]. In: StatPearls. Treasure Island (FL): StatPearls Publishing; 2024 Jan.

TTP may be either congenital or acquired. Acquired TTP is more common than the congenital type and is caused by autoantibodies targeting ADAMTS13. Antiplatelet drugs, immunosuppressive agents, HIV, estrogen-containing birth control, and pregnancy are commonly listed triggers for ADAMTS13 autoantibody formation causing acquired TTP. The less common congenital form of TTP results from mutations to ADAMTS13. In some embodiments, the subject is one diagnosed with TTP and has ADAMTS13 antibodies.

Multiple epidemiologic studies may be used to define presenting signs and symptoms for TTP, with the Oklahoma Registry being a frequently cited study. Clinical presentation of TTP according to the Oklahoma Registry includes gastrointestinal symptoms, weakness, bleeding or purpura, major neurologic findings (coma, stroke, seizure, transient focal abnormalities), minor neurologic findings (headache, confusion), fever and chills, and classical pentad comprising of hemolytic anemia, thrombocytopenia, fever, acute kidney injury, and severe neurologic findings. See, e.g., Page, Evaren E et al. “Thrombotic thrombocytopenic purpura: diagnostic criteria, clinical features, and long-term outcomes from 1995 through 2015.” Blood advances vol. 1,10 590-600. 6 Apr. 2017.

The PLASMIC score may be used to predict the likelihood of ADAMTS13 activity being less than or equal to 10% and to help make a presumptive diagnosis of TTP in the appropriate clinical setting. One point is given for each of the following features: platelet count less than 30,000/μL, presence of hemolysis (reticulocyte count greater than 2.5%, undetectable haptoglobin, or indirect bilirubin greater than 2 mg/dL), mean corpuscular volume (MCV) less than 90 fL, international normalized ratio (INR) of less than 1.5, creatinine less than 2.0 mg/dL, absence of cancer, and absence of solid organ or stem cell transplant. The higher the score, the greater the likelihood of TTP, with a score of greater than 5 suggesting a high probability of TTP and a score lower than 5 suggesting a low probability of TTP. Corticosteroids decrease autoantibody production.

The mainstay of treatment in TTP is plasma exchange with high-dose corticosteroid therapy. Other treatments used include splenectomy, cyclosporine, cyclophosphamide, vincristine, and rituximab, which are typically adjunctive agents given when first-line therapy (plasma exchange and corticosteroids) fail. Caplacizumab, a humanized monoclonal antibody fragment (a bivalent, variable-domain-only fragment) that attacks the A1 section of VWF and prevents platelet adhesion, may also be used to treat TTP.

In some embodiments, methods include selection of subjects with symptoms indicative of TTP. In some embodiments, methods include selection of subjects with symptoms indicative of relapsed TTP.

In some embodiments, the methods and use of provided CD19-directed CAR engineered cells (e.g., T cells) and/or compositions thereof, include methods for the treatment of subjects with TTP that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject to be treated relapsed to or is refractory to one or more prior therapies for treating TTP. In some embodiments, the subject to be treated is refractory to treatment with a prior therapy for treating TTP.

In any of the embodiments herein, at or immediately prior to the time of the administration of the composition comprising engineered T cells, the subject has relapsed following remission after treatment with, or become refractory to one or more prior therapies for TTP.

In any of the embodiments herein, at or immediately prior to the time of the administration of the composition comprising engineered T cells, the subject has relapsed following remission after treatment with, or become refractory to, one or more prior therapies for TTP. In any of the embodiments herein, at or immediately prior to the time of the administration of the composition comprising engineered T cells, the subject has relapsed following treatment with, or become refractory to, one or more prior therapies for the TTP. In any of the embodiments herein, the one or more prior therapies for the TTP does not comprise another dose of cells expressing the CAR. In any of the embodiments herein, the one or more prior therapies include 2 prior disease modifying therapies (DMT) with one of the prior therapies constituting an anti-CD20 antibody.

a. Response and Efficacy

In some embodiments, the provided methods and uses involving administration of an anti-CD19 CAR T cell therapy reduce TTP disease activity in the subject. In some embodiments, the reduced disease activity results in complete remission. In some embodiments, the reduced disease activity improves ADAMTS13 activity.

In some embodiments, the provided methods and uses involving administration of an anti-CD19 CAR T cell therapy improve mortality of TTP patients. In some embodiments, the provided methods and uses involving administration of an anti-CD19 CAR T cell therapy decrease the time to remission of TTP patients.

### 13. Autoimmune Hemolytic Anemia (AIHA)

Autoimmune hemolytic anemia (AIHA) is a decompensated acquired hemolysis caused by the host's immune system acting against its own red cell antigens. Classifications of AIHA include warm AIHA, cold agglutinin syndrome, paroxysmal cold hemoglobinuria, mixed-type AIHA, and drug-induced AIHA.

Once hemolysis is confirmed, further investigation is needed to establish whether that hemolysis is immune, principally by the direct anti-globulin test (DAT). The standard DAT demonstrates that immunoglobulin G (IgG) and/or complement (usually C3d) is bound to the red cell membrane. Autoantibodies may also be of IgM and IgA classes, and, in some circumstances, an extended DAT panel may be used to detect these.

Approximately 65% of patients have a warm AIHA; this may be diagnosed in patients with a consistent clinical picture and a DAT positive for IgG only or when DAT is positive for C3d ±IgG, when a clinically significant cold antibody has been excluded. Primary cold agglutinin disease (CAD) is caused by an underlying lymphoproliferative bone marrow disorder. Cold agglutinin syndrome (CAS) may be diagnosed in patients with laboratory criteria consistent with a clinically significant cold antibody that occurs in association with secondary disorders, such as infection, SLE, or aggressive lymphoma. Mixed AIHA is caused by a combination of a warm IgG antibody and a cold IgM antibody. The DAT is usually positive for IgG and C3d. Paroxysmal cold hemoglobinuria (PCH) usually occurs in children.

Splenectomy, steroids, monoclonal antibodies (e.g., rituximab), or immunosuppressants have been used as treatment options for autoimmune hemolytic anemia. Patients who are steroid sensitive, but refractory or relapsing after third-line therapies, may tolerate longer-term treatment with low-dose prednisolone (≤10 mg), which may be used in conjunction with a steroid-sparing agent.

Patients who are diagnosed with autoimmune hemolytic anemia with marked anemia at onset are at increased risk of multiple relapses as well as more often seen to be refractory to multiple lines of treatment.

See, e.g., Gehrs, Bradley C, and Richard C Friedberg. “Autoimmune hemolytic anemia.” American journal of hematology vol. 69,4 (2002): 258-71 and Hill, Anita, and Quentin A Hill. “Autoimmune hemolytic anemia.” Hematology. American Society of Hematology. Education Program vol. 2018,1 (2018): 382-389.

In some embodiments, methods include selection of subjects with symptoms indicative of AIHA. In some embodiments, methods include selection of subjects with symptoms indicative of relapsed AIHA. In some embodiments, the subject relapsed to or is refractory to one or more prior therapies for treating AIHA. In some embodiments, the subject is refractory to treatment with a prior therapy for treating AIHA.

In some embodiments, the methods and use of provided CD19-directed CAR engineered cells (e.g., T cells) and/or compositions thereof, include methods for the treatment of subjects with AIHA that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19.

In any of the embodiments herein, at or immediately prior to the time of the administration of the composition comprising engineered T cells, the subject has relapsed following remission after treatment with, or become refractory to one or more prior therapies for AIHA.

In any of the embodiments herein, at or immediately prior to the time of the administration of the composition comprising engineered T cells, the subject has relapsed following remission after treatment with, or become refractory to, one or more prior therapies for AIHA. In any of the embodiments herein, at or immediately prior to the time of the administration of the composition comprising engineered T cells, the subject has relapsed following treatment with, or become refractory to, one or more prior therapies for the AIHA. In any of the embodiments herein, the one or more prior therapies for the AIHA does not comprise another dose of cells expressing the CAR. In any of the embodiments herein, the one or more prior therapies include 2 prior disease modifying therapies (DMT) with one of the prior therapies constituting an anti-CD20 antibody. a. Response and Efficacy

In some embodiments, the provided methods and uses involving administration of an anti-CD19 CAR T cell therapy reduce AIHA disease activity in the subject. In some embodiments, the reduced disease activity results in complete remission. In some embodiments, subjects administered an anti-CD19 CAR T cell therapy achieve a complete response.

In some embodiments, the provided methods and uses involving administration of an anti-CD19 CAR T cell therapy improve mortality of AIHA patients. In some embodiments, the provided methods and uses involving administration of an anti-CD19 CAR T cell therapy decrease the time to remission of AIHA patients.

### 14. Immune Thrombocytopenia (ITP)

Immune thrombocytopenia (ITP), formerly idiopathic thrombocytopenic purpura, is an autoimmune disorder characterized by a low platelet count, purpura, and hemorrhagic episodes. ITP arises from immunoglobulin G (IgG) autoantibodies sensitizing circulating platelets. The American Society of Hematology (ASH) defines ITP as a generalized purpuric rash accompanied by a platelet count less than 100,000/L and normal white blood cell (WBC) count and hemoglobin level.

ITP without an underlying disorder is known as primary ITP. Secondary ITP typically has an external cause, including drugs and other autoimmune conditions like systemic lupus erythematosus. Primary ITP may be further categorized based on the timing and persistence of symptoms. Newly diagnosed ITP refers to the condition from the time of diagnosis to 3 months afterward. Persistent ITP arises when symptoms continue 3 to 12 months following the initial diagnosis. Chronic ITP (cITP) indicates ongoing symptoms beyond 12 months from the initial diagnosis until resolution or further management. Refractory ITP includes cases that do not resolve with splenectomy. Severe ITP, when platelet counts are below 20,000/L, warrants medical treatment.

When symptomatic, mucocutaneous bleeding is a common manifestation, ranging from mild petechiae and epistaxis to severe and potentially life-threatening gastrointestinal, intracranial, or urinary tract hemorrhage. Bruising or a petechial rash may be reported. Risk factors for major bleeding include a platelet count of less than 20,000/L, advanced age, and prior minor bleeding, with a higher incidence in the months following acute ITP diagnosis.

ASH guidelines recommend corticosteroids over observation in adults with newly diagnosed ITP and a platelet count of less than 30,000/L if they are asymptomatic or have minor mucocutaneous bleeding. In adults with ITP, the ASH guideline panel recommends second-line therapies if they are dependent on corticosteroids, unresponsive to corticosteroids for at least 3 months, or considered to have chronic ITP (cITP). Second-line treatments for adults include TPO-RAs (i.e., eltrombopag, romiplostim, or avatrombopag0), rituximab, and splenectomy. Rituximab is typically administered at a dose of 375 mg/m IV once a week for 4 consecutive weeks. This drug is contraindicated in patients with hepatitis B infection, as it risks progression to fulminant hepatitis, liver failure, and mortality. Efgartigimod and rozanolixizumab are compounds undergoing testing for use in ITP. These agents are antibody fragments targeting the neonatal Fc-receptor (FcRn), thus inhibiting IgG recycling. Efgartigimod and rozanolixizumab decrease IgG's half-life, reducing it to normal or subpathogenic levels. See, e.g., Pietras N M, Gupta N, Justiz Vaillant A A, et al. Immune Thrombocytopenia. [Updated 2024 May 5]. In: StatPearls. Treasure Island (FL): StatPearls Publishing; 2024 Jan.

In some embodiments, the provided methods and uses involving administration of an anti-CD19 CAR T cell therapy reduce ITP disease activity in the subject. In some embodiments, methods include selection of subjects with symptoms indicative of ITP. In some embodiments, methods include selection of subjects with symptoms indicative of relapsed ITP.

In some embodiments, the methods and use of provided CD19-directed CAR engineered cells (e.g., T cells) and/or compositions thereof, include methods for the treatment of subjects with ITP that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject is diagnosed with ITP and has detectable platelet-targeted antibodies. In some embodiments, the subject relapsed to or is refractory to one or more prior therapies for treating ITP. In some embodiments, the subject is refractory to treatment with a prior therapy for treating ITP.

In some embodiments, the provided methods and uses involving administration of an anti-CD19 CAR T cell therapy reduce ITP disease activity in the subject.

In any of the embodiments herein, at or immediately prior to the time of the administration of the composition comprising engineered T cells, the subject has relapsed following remission after treatment with, or become refractory to, one or more prior therapies for ITP. In any of the embodiments herein, at or immediately prior to the time of the administration of the composition comprising engineered T cells, the subject has relapsed following treatment with, or become refractory to, one or more prior therapies for the ITP. In any of the embodiments herein, the one or more prior therapies for the ITP does not comprise another dose of cells expressing the CAR. In any of the embodiments herein, the one or more prior therapies include 2 prior disease modifying therapies (DMT) with one of the prior therapies constituting an anti-CD20 antibody.

In some embodiments, the subject to be treated in accord with provided methods is characterized as having or having had ITP that last 6 months or longer. Typically acute ITP resolves within 6 months (usually spontaneously), persistent ITP lasts 3-12 months, chronic lasts more than 12 months.

In some embodiments, the subject has chronic ITP (cITP). ITP is characterized as cITP where there are ongoing symptoms beyond 12 months from the initial diagnosis until resolution or further management. When symptomatic, mucocutaneous bleeding is a common manifestation, ranging from mild petechiae and epistaxis to severe and potentially life-threatening gastrointestinal, intracranial, or urinary tract hemorrhage. Bruising or a petechial rash may be reported. Risk factors for major bleeding include a platelet count of less than 20,000/μL, advanced age.

Existing therapies for treating cITP include corticosteroids or other second-line therapies. ASH guidelines recommend corticosteroids over observation in adults with chronic ITP and a platelet count of less than 30,000/L if they are asymptomatic or have minor mucocutaneous bleeding. In adults with cITP, the ASH guideline panel recommends second-line therapies. Second-line treatments for adults include thrombopoietin receptor agonists (TPO-RAs; e.g., eltrombopag, romiplostim, or avatrombopag), an anti-CD20 antibody (e.g., rituximab), and splenectomy. Rituximab is typically administered at a dose of 375 mg/m IV once a week for 4 consecutive weeks. This drug is contraindicated in patients with hepatitis B infection, as it risks progression to fulminant hepatitis, liver failure, and mortality. Efgartigimod and rozanolixizumab are compounds undergoing testing for use in ITP. These agents are antibody fragments targeting the neonatal Fc-receptor (FcRn), thus inhibiting IgG recycling. Efgartigimod and rozanolixizumab decrease IgG's half-life, reducing it to normal or subpathogenic levels. See, e.g., Pietras N M, Gupta N, Justiz Vaillant A A, et al. Immune Thrombocytopenia. [Updated 2024 May 5]. In: StatPearls. Treasure Island (FL): StatPearls Publishing; 2024 Jan.

In some embodiments, the methods described herein are useful for treating subjects having cITP. In some embodiments, the subject may have ITP persisting for greater than twelve months and characterized by immune-mediated destruction of platelets and impaired platelet production. In some embodiments, the subject has persistently low platelet counts (e.g., less than about 100×10⁹/L, and in severe cases less than about 30×10⁹/L). Patients with cITP frequently present with bleeding manifestations such as petechiae, purpura, epistaxis, gingival bleeding, or menorrhagia, and in some cases may develop gastrointestinal or intracranial hemorrhage. Disease severity may be assessed based on platelet count, clinical bleeding scores (e.g., World Health Organization bleeding scale or ITP Bleeding Score), and prior bleeding history.

In some embodiments, the provided methods and uses involving administration of an anti-CD19 CAR T cell therapy reduce cITP disease activity in the subject. In some embodiments, methods include selection of subjects with symptoms indicative of cITP. In some embodiments, methods include selection of subjects with symptoms indicative of relapsed cITP.

In some embodiments, the methods and use of provided CD19-directed CAR engineered cells (e.g., T cells) and/or compositions thereof, include methods for the treatment of subjects with cITP that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject is diagnosed with cITP and has detectable platelet-targeted antibodies. In some embodiments, the subject relapsed to or is refractory to one or more prior therapies for treating cITP. In some embodiments, the subject is refractory to treatment with a prior therapy for treating cITP. In some embodiments, the patient population includes subjects who have relapsed following, or are refractory to, one or more prior therapies, including but not limited to corticosteroids, intravenous immunoglobulin (IVIG), anti-D immunoglobulin, splenectomy, rituximab, thrombopoietin receptor agonists (e.g., romiplostim, eltrombopag, avatrombopag), and/or other immunosuppressive agents.

In some embodiments, the provided methods and uses involving administration of an anti-CD19 CAR T cell therapy reduce cITP disease activity in the subject.

In any of the embodiments herein, at or immediately prior to the time of the administration of the composition comprising engineered T cells, the subject has relapsed following remission after treatment with, or become refractory to, one or more prior therapies for cITP. In any of the embodiments herein, at or immediately prior to the time of the administration of the composition comprising engineered T cells, the subject has relapsed following treatment with, or become refractory to, one or more prior therapies for the cITP. In any of the embodiments herein, the one or more prior therapies for the cITP does not comprise another dose of cells expressing the CAR. In any of the embodiments herein, the one or more prior therapies include 2 prior disease modifying therapies (DMT) with one of the prior therapies constituting an anti-CD20 antibody. In some embodiments, the one or more prior therapies includes a TPO-RA. a. Response and Efficacy

In some embodiments, the provided methods and uses involving administration of an anti-CD19 CAR T cell therapy reduce ITP disease activity in the subject. In some embodiments, the reduced disease activity results in complete remission. In some embodiments, the reduced disease activity results in platelet count>100×10⁹/L and an absence of bleeding.

In some embodiments, the provided methods and uses involving administration of an anti-CD19 CAR T cell therapy improve mortality of ITP patients. In some embodiments, the provided methods and uses involving administration of an anti-CD19 CAR T cell therapy decrease the time to remission of ITP patients.

In some embodiments, the provided methods and uses involving administration of an anti-CD19 CAR T cell therapy reduce cITP disease activity in the subject. In some embodiments, the reduced disease activity results in complete remission. In some embodiments, the reduced disease activity results in platelet count>100×10⁹/L and an absence of bleeding.

In some embodiments, the provided methods and uses involving administration of an anti-CD19 CAR T cell therapy improve mortality of cITP patients. In some embodiments, the provided methods and uses involving administration of an anti-CD19 CAR T cell therapy decrease the time to remission of cITP patients. 15. IgA Nephropathy (IgA)

Immunoglobulin A (IgA) nephropathy, or IgAN, also known as Berger disease, is one of the leading causes of glomerulonephritis and renal failure. This disease is a prevalent form of glomerulonephritis characterized by the deposition of IgA in the glomerular basement membrane. Immune-mediated damage to the basement membrane results in hematuria, proteinuria, and renal insufficiency. Pathologically, a spectrum of glomerular lesions may be seen, with mesangial proliferation and prominent IgA deposition being a commonly observed change.

Many systemic conditions are associated with IgA deposits, causing IgAN pathology. Recent reports suggest the pathways leading to glomerular injury are similar between primary and secondary IgAN. Secondary IgAN may arise from various conditions, including liver, gastrointestinal, autoimmune, dermatological, infectious, and drug-related causes. Autoimmune disorders, including Sjagren syndrome, spondyloarthritis, systemic lupus erythematosus, and Behget disease, have been associated with the development of IgA.

Although IgAN is a common cause of glomerulonephritis, its exact prevalence remains uncertain due to many cases being asymptomatic and requiring a renal biopsy for definitive diagnosis. Histologically, IgAN is characterized by the following characteristics: diffuse proliferation of mesangial cells and matrix, hypercellular or normal glomeruli with diffuse necrotizing crescentic glomerulonephritis, mesangial involvement resembling focal and segmental glomerulosclerosis, and immunofluorescence reveals a diffuse granular pattern of IgA deposits in the mesangium.

In many patients with IgAN, history and physical examination are typically unremarkable. The primary complaint often revolves around gross hematuria, which may manifest as brown or red urine. Acute renal failure may lead to ankle edema, facial puffiness, and hypertension. During a physical examination, it is essential to assess blood pressure and observe for indications of diminished renal function, including edema, ascites, and basal lung crepitations. Hypertension is a common manifestation in IgAN. In addition, IgAN may coexist with cirrhosis, liver diseases, and celiac disease. Progressive chronic kidney disease (CKD) is frequently observed in various cohorts. See, e.g., Rout P, Limaiem F, Hashmi M F. IgA Nephropathy (Berger Disease) [Updated 2024 Apr 22]. In: StatPearls. Treasure Island (FL): StatPearls Publishing; 2024 Jan.

To evaluate a subject for IgA, proteinuria is assessed using the protein-to-creatinine ratio in urine or 24-hour urinary protein excretion. Serum creatinine and estimated GFR are also measured to quantify renal function. Confirmation of the diagnosis relies on renal biopsy.

Treatment of IgA considers the assessed proteinuria, eGFR, blood pressure, and histological findings. The primary goals of treatment are to induce remission and avert the onset of complications. First-line agents for managing proteinuria and lowering blood pressure in IgAN patients typically involve angiotensin-converting enzyme (ACE) inhibitors or angiotensin receptor blockers (ARBs). The target blood pressure is typically set at 130/80 mm Hg. If proteinuria is more than 1 g/d, the systolic blood pressure target is less than 125 mm Hg.

Corticosteroids are crucial in reducing proteinuria in IgAN, particularly when levels exceed lg/d. Steroid administration has especially been shown to decrease proteinuria. Typically, a tapering course of prednisone is prescribed for 2 to 4 months. Other medications may be used to treat IgA including, sparsentan (i.e., a non-immunosuppressive, novel dual endothelin and angiotensin receptor antagonist), budesonide (i.e., in the form of oral nefecon), and sodium-glucose cotransporter-2 (SGLT2) inhibitors. Other immunosuppressive agents to use in the treatment of IgA include mycophenolate mofetil (MMF), rituximab, cyclophosphamide, azathioprine, calcineurin inhibitors, and leflunomide. Tonsillectomy and renal transplants are surgical treatments of IgA.

In some embodiments, the provided methods and uses involving administration of an anti-CD19 CAR T cell therapy reduce IgA disease activity in the subject. In some embodiments, methods include selection of subjects with symptoms indicative of IgA. In some embodiments, methods include selection of subjects with symptoms indicative of relapsed IgA.

In some embodiments, the methods and use of provided CD19-directed CAR engineered cells (e.g., T cells) and/or compositions thereof, include methods for the treatment of subjects with IgA that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject relapsed to or is refractory to one or more prior therapies for treating IgA. In some embodiments, the subject is refractory to treatment with a prior therapy for treating IgA. In some embodiments, the subject to be treated has been diagnosed with IgA Nephropathy and is/has: high-risk proteinuria (>lg/g), refractory to one or two prior lines of therapy (including GS, immunosuppressants, RAAS), advancing to end-stage kidney disease (ESKD). See, e.g., Lerma, Edgar V et al. “Impact of Proteinuria and Kidney Function Decline on Health Care Costs and Resource Utilization in Adults With IgA Nephropathy in the United States: A Retrospective Analysis.” Kidney medicine vol. 5,9 100693. 25 Jun. 2023, doi:10.1016/j.xkme.2023 and Uriol-Rivera, M. G., Obrador-Mulet, A., Julii, M. R. et al. Sequential administration of paricalcitol followed by IL-17 blockade for progressive refractory IgA nephropathy patients. Sci Rep 14, 4866 (2024).

In any of the embodiments herein, at or immediately prior to the time of the administration of the composition comprising engineered T cells, the subject has relapsed following remission after treatment with, or become refractory to, one or more prior therapies for IgA. In any of the embodiments herein, at or immediately prior to the time of the administration of the composition comprising engineered T cells, the subject has relapsed following treatment with, or become refractory to, one or more prior therapies for the IgA. In any of the embodiments herein, the one or more prior therapies for the IgA does not comprise another dose of cells expressing the CAR. In any of the embodiments herein, the one or more prior therapies include 2 prior disease modifying therapies (DMT) with one of the prior therapies constituting an anti-CD20 antibody.

a. Response and Efficacy

In some embodiments, the provided methods and uses involving administration of an anti-CD19 CAR T cell therapy reduce IgA disease activity in the subject. In some embodiments, the reduced disease activity results in complete remission. In some embodiments, the reduced disease activity results in decreased levels of proteinuria. In some embodiments, the treated subjects do not see progression to ESKD.

In some embodiments, the provided methods and uses involving administration of an anti-CD19 CAR T cell therapy improve mortality of IgA patients. In some embodiments, the provided methods and uses involving administration of an anti-CD19 CAR T cell therapy decrease the time to remission of IgA patient.

### 16. Bullous Pemphigoid (BP)

Bullous pemphigoid (BP) is a common autoimmune subepidermal blistering disorder, representing 80% of subepidermal immunobullous cases. Bullous pemphigoid commonly affects elderly patients between the ages of 60 to 80 years. While the clinical presentation of bullous pemphigoid is broad, the immunobullous skin disorder characteristically presents with tense bullae and intense generalized pruritus. In atypical cases, bullous lesions may be absent, and these cases require a high degree of clinical suspicion. A biopsy for hematoxylin and eosin staining will show a subepidermal split with eosinophils, and direct immunofluorescence will highlight the autoantibodies against the basement membrane zone. ELISA testing is also useful in diagnosing bullous pemphigoid. Treatment depends on the severity of the disease; however, standard therapies involve topical or systemic immunosuppressive agents. Prognosis varies, and long-term monitoring is often required.

There are two main components to the pathophysiology of BP: immunologic and inflammatory. The immunologic elements comprise autoantibodies against two parts of the basal keratinocyte hemidesmosomal proteins BP antigen 230 (BPAG1) and BP antigen 180 (BPAG2 or type XVII collagen). These antigens play an essential part in the adhesion complexes that promote epithelial-stromal adhesion. When autoantibodies bind to their target antigen, the inflammatory component follows, activating complement and mast cells. This causes neutrophils and eosinophils to release various inflammatory cells resulting in the release of proteolytic enzymes that damage the dermal-epidermal junction.

Diagnosis of the bullous pemphigoid relies on the clinical scenario and laboratory tests. Histology with direct and indirect immunofluorescence studies aid in diagnosis. Histology will show a subepidermal split with a superficial perivascular inflammatory infiltrate and numerous eosinophils. Spongiosis and superficial papillary dermal infiltrate of eosinophils without vesiculation characterize urticarial lesions and may provide a clue to the dermatopathologist for diagnosing urticarial or pre-bullous pemphigoid. ELISA testing to detect antibodies to the NC16A domain of BP180, also known as BPAG2, is available and has a sensitivity of 89% and specificity of 98%. Autoantibodies to BP180 and BP230 may be identified in normal subjects without bullous pemphigoid, but they will not bind to the NC16A domain. Serum IgG autoantibodies levels to BP180 have been shown to correlate with disease severity in several ELISA-based studies. Also, a high BP180-NC16A ELISA score and positive direct immunofluorescence at the end of therapy show possible relapse.

The mainstay of treatment for bullous pemphigoid is systemic corticosteroids, but treatment ultimately depends on comorbidities and the extent of the disease. For localized disease, less than 20% body surface area in an elderly patient, super-potent topical steroids such as clobetasol may be used. Topical steroids combined with nicotinamide plus tetracycline, minocycline, or doxycycline have shown success in multiple cases.

For extensive disease, systemic prednisone at a dose of 0.5 to 1.0 mg/kg per day is recommended. This dose of systemic corticosteroids controls the disease within approximately two weeks and may be slowly tapered over six to nine months or longer. This treatment regimen is limited by patient age, comorbidities, and side effects. Potent topical corticosteroids may control generalized BP with fewer systemic side effects.

Immunosuppressive therapy is used when steroids do not control the disease or if patients have contraindications for systemic corticosteroid treatments. Alternative agents include azathioprine, mycophenolate mofetil, methotrexate, chlorambucil, and cyclophosphamide. If all other treatments fail, IVIG, anti-CD20 (rituximab), or omalizumab may be used for treatment-resistant cases.

There are several observation endpoints that may be used to evaluate subjects in early, intermediate, and late stages. During the intermediate stage, the corticosteroids and other treatments are usually being tapered, but for some patients medication doses do not change because of flaring with attempts to taper treatment. Complete remission during tapering is the absence of non-transient lesions while the patient is receiving more than minimal therapy. There is no minimum time point here as the patient is under control but has not yet reached the desired outcome of disease remission on minimal or no therapy. Late observation end points of disease activity are identified as: (1) complete remission off therapy; and (2) complete remission on therapy, both of which only apply to patients who have had no new or established lesions for at least 2 months. “Complete remission off therapy” is defined as an absence of new or established lesions or pruritic symptoms while the patient is off all BP therapy for at least 2 months. See, e.g., Murrell, Dedee F et al. “Definitions and outcome measures for bullous pemphigoid: recommendations by an international panel of experts.” Journal of the American Academy of Dermatology vol. 66,3 (2012): 479-85.

BP Disease Area Index (BPDAI) may also be used to measure disease activity. BPDAI measures skin and mucosal involvement with two activity components, one for extent of blistering, one for extent of urticarial/eczematous change and a separate damage component. BPDAI scores below 20 are classified as mild disease, while scores of 57 or higher indicate high disease severity.

In some embodiments, the provided methods and uses involving administration of an anti-CD19 CAR T cell therapy reduce BP disease activity in the subject. In some embodiments, methods include selection of subjects with symptoms indicative of BP. In some embodiments, methods include selection of subjects with symptoms indicative of relapsed BP.

In some embodiments, the methods and use of provided CD19-directed CAR engineered cells (e.g., T cells) and/or compositions thereof, include methods for the treatment of subjects with BP that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to CD19. In some embodiments, the subject relapsed to or is refractory to one or more prior therapies for treating BP. In some embodiments, the subject is refractory to treatment with a prior therapy for treating BP. In some embodiments, the subject to be treated has been diagnosed with Bullous Pemphigoid and is/has: BPDAI>57, and/or relapsed or refractory to one prior line of therapy.

In some embodiments, the provided methods and uses involving administration of an anti-CD19 CAR T cell therapy reduce BP disease activity in the subject.

In any of the embodiments herein, at or immediately prior to the time of the administration of the composition comprising engineered T cells, the subject has relapsed following remission after treatment with, or become refractory to, one or more prior therapies for IgA. In any of the embodiments herein, at or immediately prior to the time of the administration of the composition comprising engineered T cells, the subject has relapsed following treatment with, or become refractory to, one or more prior therapies for the BP. In any of the embodiments herein, the one or more prior therapies for the BP does not comprise another dose of cells expressing the CAR. In any of the embodiments herein, the one or more prior therapies include 2 prior disease modifying therapies (DMT) with one of the prior therapies constituting an anti-CD20 antibody.

a. Response and Efficacy

In some embodiments, the provided methods and uses involving administration of an anti-CD19 CAR T cell therapy reduce BP disease activity in the subject. In some embodiments, the reduced disease activity results in complete remission. In some embodiments, the reduced disease activity results in decreased levels of BP180. In some embodiments, the reduced disease activity results in a decrease in BPDAI score.

In some embodiments, the provided methods and uses involving administration of an anti-CD19 CAR T cell therapy improve mortality of BP patients. In some embodiments, the provided methods and uses involving administration of an anti-CD19 CAR T cell therapy decrease the time to remission of BP patients.

### 17. Ulcerative Colitis (UC)

Ulcerative colitis (UC) is a chronic, relapsing inflammatory bowel disease (IBD) characterized by diffuse mucosal inflammation limited to the colon. The typical clinical presentation includes persistent diarrhea, often with blood and mucus, abdominal pain, urgency, and tenesmus. Patients may also experience systemic symptoms such as fatigue, weight loss, and, in severe cases, fever. The disease course is marked by periods of exacerbation and remission. Extraintestinal manifestations, including arthralgias, skin lesions (e.g., erythema nodosum), and ocular inflammation, may also occur in a subset of patients.

The pathogenesis of UC involves a complex interplay between genetic susceptibility, environmental factors, the intestinal microbiota, and immune dysregulation. The inflammatory process is typically limited to the mucosal layer of the colon and rectum, beginning in the rectum and extending proximally in a continuous fashion. Aberrant immune responses, particularly involving T-helper cells and cytokines such as tumor necrosis factor-alpha (TNF-α), contribute to the chronic inflammation and tissue injury observed in UC. Mechanistically, TNF-α contributes to the disruption of the intestinal epithelial barrier by inducing apoptosis of epithelial cells and altering tight junction proteins, which exacerbates mucosal damage and perpetuates the inflamatory cycle In UC, there is an abnormal activation of the mucosal immune system, particularly involving CD4+ T helper (Th) cells. Unlike Crohn's disease, which is associated with a Th1/Th17 response, UC is traditionally associated with a Th2-like immune response. However, the Th2 response in UC is atypical, as it is characterized by the production of cytokines such as interleukin-5 (IL-5) and interleukin-13 (IL-13), rather than the classic Th2 cytokine interleukin-4 (IL-4).

IL-13, in particular, has been implicated in the pathogenesis of UC. It is produced by a subset of natural killer T (NKT) cells and can induce epithelial cell apoptosis and disrupt tight junctions, leading to increased intestinal permeability and mucosal injury. Additionally, regulatory T cells (Tregs), which normally function to suppress excessive immune responses, may be functionally impaired in UC, further contributing to chronic inflammation.

The diagnosis of UC is based on a combination of clinical, endoscopic, histologic, and laboratory findings. Laboratory tests commonly used include: Complete Blood Count (CBC), which may reveal anemia and leukocytosis, C-reactive Protein (CRP) and Erythrocyte Sedimentation Rate (ESR), which are elevated in active inflammation, Stool Studies, which are used to exclude infectious etiologies (e.g., Clostridioides difficile, bacterial pathogens), Fecal Calprotectin or Lactoferrin. Endoscopic evaluation (flexible sigmoidoscopy or colonoscopy) with mucosal biopsies is essential for confirmation, demonstrating continuous mucosal inflammation and crypt architectural distortion.

The cornerstone of UC management is medical therapy aimed at inducing and maintaining remission. First-line treatments for mild to moderate disease include aminosalicylates (e.g., mesalamine) administered orally or rectally. For moderate to severe disease, corticosteroids (oral or intravenous) are used for induction of remission but are often not suitable for long-term maintenance due to adverse effects. Immunomodulators such as azathioprine or 6-mercaptopurine may be used for steroid-sparing maintenance therapy.

In patients with refractory or severe disease, biologic agents targeting specific inflammatory pathways are considered. These include anti-TNF agents (e.g., infliximab, adalimumab), anti-integrin therapies (e.g., vedolizumab), and anti-interleukin-12/23 agents (e.g., ustekinumab). Janus kinase (JAK) inhibitors (e.g., tofacitinib) are also approved for moderate to severe UC. Surgical intervention (colectomy) is reserved for patients with medically refractory disease, dysplasia, or complications such as toxic megacolon.

Observation endpoints in UC management include clinical remission (resolution of symptoms), endoscopic healing (mucosal healing), histologic remission, and normalization of inflammatory markers. Regular monitoring is essential to assess disease activity, guide therapy, and screen for complications such as colorectal cancer. Tests to measure disease activity include the Mayo Score, the Simple Clinical Colitis Activity index (SCCAI), Mayo Endoscopic Subscore, Ulcerative Colitis Endoscopic Index of Severity (UCEIS) Total Score, Geboes Score, Robarts Histopathology Index (RHI), and combinations thereof.

The Mayo score, also known as the Mayo Clinic Score, can be used to measure disease activity and is a composite clinical index widely used to assess disease activity in ulcerative colitis (UC). It incorporates four components: stool frequency, rectal bleeding, endoscopic findings, and the physician's global assessment. Each component is scored from 0 to 3, resulting in a total score ranging from 0 to 12. Higher scores indicate more severe disease activity. The Mayo score is frequently used in clinical trials and practice to evaluate response to therapy, with specific thresholds defining remission, mild, moderate, and severe disease.

The Simple Clinical Colitis Activity Index (SCCAI) is another validated tool for assessing UC disease activity, particularly in outpatient settings. The SCCAI is based on patient-reported symptoms over the preceding week, including bowel frequency during the day and at night, urgency of defecation, presence of blood in stool, general well-being, and the presence of extracolonic manifestations. Each parameter is assigned a score, and the total score helps categorize disease activity as remission, mild, moderate, or severe.

The Geboes Score is comprised of seven categories (or grades), each of which describes a histologic item, including “structural (architectural change)” (grade 0), “chronic inflammatory infiltrate” (grade 1), “lamina propria eosinophils” (grade 2A), “lamina propria neutrophils” (grade 2B), “neutrophils in epithelium” (grade 3), “crypt destruction” (grade 4) and “surface epithelial injury”(grade 5). Each grade includes subscores that indicate the degree of abnormality seen for that histologic characteristic, with subscores of 0 indicating normal appearance and higher subscores indicating increasingly abnormal appearance. The RHI uses the weighted results from 4 Geboes score categories (“chronic inflammatory infiltrate”, “lamina propria neutrophils”, “neutrophils in epithelium” and “surface epithelial injury”) to derive a continuous score, ranging from 0 (no disease activity) to 33 (severe disease activity). The RHI was developed as a responsive instrument to detect treatment effects in early drug development.

In some embodiments, the provided methods and uses involving administration of an anti-CD19 CAR T cell therapy reduces UC disease activity in the subject. In some embodiments, methods include selection of subjects with symptoms indicative of UC. In some embodiments, the subject is diagnosed with UC. In particular embodiments, the method includes administering to the subject a dose of T cells that includes CD4+ and CD8+ T cells, wherein the T cells comprise a chimeric antigen receptor (CAR) that specifically binds to CD19.

In some embodiments, the methods described herein are useful for treating subjects having severe ulcerative colitis (UC). In provided aspects, severe UC may be characterized by frequent bloody stools (e.g., ≥6 per day) in combination with one or more systemic features such as fever, anemia, tachycardia, elevated erythrocyte sedimentation rate (ESR), or elevated C-reactive protein (CRP), consistent with Truelove and Witts' criteria. Severe UC may also be defined by a high Mayo score (e.g., 11-12) or elevated Ulcerative Colitis Endoscopic Index of Severity (UCEIS), and may be further characterized by objective markers of inflammation including elevated fecal calprotectin and hypoalbuminemia. Patients with severe UC often present with clinical symptoms such as urgency, tenesmus, abdominal pain, fatigue, and systemic manifestations including weight loss and malaise. In certain embodiments, the patient population includes subjects who have relapsed following, or are refractory to, one or more prior therapies, including but not limited to 5-aminosalicylates, corticosteroids (including corticosteroid-refractory or corticosteroid-dependent disease), immunomodulators (e.g., azathioprine or 6-mercaptopurine), biologics (e.g., anti-TNF agents, anti-integrin agents, or anti-IL-12/23 agents), and/or small molecule therapies such as Janus kinase (JAK) inhibitors.

In some embodiments, methods include selection of subjects with symptoms indicative of relapsed UC. In some embodiments, the methods and use of provided CD19-directed CAR engineered cells (e.g., T cells) and/or compositions thereof, include methods for the treatment of subjects with UC that have failed at least one or more prior therapies, such as any described above. In some embodiments, the subject relapsed to or is refractory to one or more prior therapies for treating UC. In some embodiments, the subject is refractory to treatment with a prior therapy for treating UC. In any of the embodiments herein, at or immediately prior to the time of the administration of the composition comprising engineered T cells, the subject has relapsed following remission after treatment with, or become refractory to, one or more prior therapies for UC. In any of the embodiments herein, at or immediately prior to the time of the administration of the composition comprising engineered T cells, the subject has relapsed following treatment with, or become refractory to, one or more prior therapies for the UC.

In some embodiments, the methods and uses of provided CD19-directed CAR engineered cells (e.g., T cells) and/or compositions thereof, include methods for the treatment of subjects with UC that have failed one or more prior therapies, such as have relapsed after initially responding to treatment with a prior therapy or is refractory and never responded to a prior therapy. In some embodiments, the methods and use thereof includes treatment of subjects with UC that have failed aminosalicylates (e.g., mesalamine). In some embodiments, the methods and use thereof includes treatment of subjects with UC that have failed corticosteroids (e.g., prednisone or budesonide). In some embodiments, the methods and use thereof includes treatment of subjects with UC that have failed immunomodulators (e.g., azathioprine or 6-mercaptopurine). In some embodiments, the methods and use thereof includes treatment of subjects with UC that have failed biologic agents targeting specific inflammatory pathways (e.g., anti-TNF agents like infliximab or adalimumab, anti-integrin agents like vedolizumab, or anti-IL-12/23 agents, e.g., ustekinumab). In some embodiments, the methods and use thereof includes treatment of subjects with UC that have failed small molecule therapies, e.g., Janus kinase (JAK) inhibitors (e.g., tofacitinib).

a. Response and Efficacy

In some embodiments, the provided methods and uses involving administration of an anti-CD19 CAR T cell therapy reduce UC disease activity in the subject. In some embodiments, the reduced disease activity results in prognostic of at least one or more of clinical response, clinical remission, endoscopic remission, endoscopic healing, symptomatic remission, improvement in endoscopic histologic inflammation, corticosteroid free remission, bowel urgency remission, improvement in bowel urgency, stool frequency remission, improvement in stool frequency, improvement in fatigue. In some embodiments, the reduced disease activity results in a lower Mayo Score, the Simple Clinical Colitis Activity index (SCCAI), Mayo Endoscopic Subscore, Ulcerative Colitis Endoscopic Index of Severity (UCEIS) Total Score, Geboes Score, Robarts Histopathology Index (RHI). In some embodiments, the reduced disease activity results in a lower Mayo Score. In some embodiments, the reduced disease activity results in less abdominal pain, less diarrhea, and/or less constipation.

In some embodiments, the provided methods and uses involving administration of an anti-CD19 CAR T cell therapy improve mortality of UC patients. In some embodiments, the provided methods and uses involving administration of an anti-CD19 CAR T cell therapy decrease the time to remission of UC patients.

### B. Dosing

In some embodiments, a dose of engineered cells is administered to subjects in accordance with the provided methods, and/or with the provided articles of manufacture or compositions. In some embodiments, engineered cells are for use in the manufacture of a medicament. In some embodiments, the size or timing of the doses is determined as a function of the particular disease or condition in the subject. In some cases, the size or timing of the doses for a particular disease in view of the provided description may be empirically determined.

In some of any of the provided embodiments, the dose of T cells, such as engineered T cells expressing a recombinant receptor, includes is enriched for, or comprises a cell composition or a cell population that is enriched for, CD3+ T cells, CD4+ T cells, CD8+ T cells or CD4+ T cells and CD8+ T cells. In some of any such embodiments, greater than at or about 70%, 75%, 80%, 85%, 90%, 95% or 98% of the cells in the dose of T cells are CD3+ T cells, CD4+ T cells, CD8+ T cells or CD4+ T cells and CD8+ T cells. In some of any such embodiments, greater than at or about 70%, 75%, 80%, 85%, 90%, 95% or 98% of the cells in the dose of T cells are CD3+ T cells. In some of any of the provided embodiments, the dose of T cells comprises both CD4+ cells and CD8+ cells. In some of any such embodiments, greater than at or about 70%, 75%, 80%, 85%, 90%, 95% or 98% of the cells in the dose of T cells are CD4+ T cells and CD8+ T cells.

In some embodiments, the dose of cells comprises between at or about 0.1×10⁵ of the CD19-directed CAR engineered cells per kilogram body weight of the subject (cells/kg) and at or about 2×10⁶ cells/kg, such as between at or about 0.1×10⁵ cells/kg and at or about 0.5×10⁵ cells/kg, between at or about 0.5×10⁵ cells/kg and at or about 1×10⁵ cells/kg, between at or about 1×10⁵ cells/kg and at or about 1.5×10⁵ cells/kg, between at or about 1.5×10⁵ cells/kg and at or about 2×10⁵ cells/kg, between at or about 2×10⁵ cells/kg and at or about 2.5×10⁵ cells/kg, between at or about 2.5×10⁵ cells/kg and at or about 3×10⁵ cells/kg, between at or about 3×10⁵ cells/kg and at or about 3.5×10⁵ cells/kg, between at or about 3.5×10⁵ cells/kg and at or about 4×10⁵ cells/kg, between at or about 4×10⁵ cells/kg and at or about 4.5×10⁵ cells/kg, between at or about 4.5×10⁵ cells/kg and at or about 5×10⁵ cells/kg, between at or about 5×10⁵ cells/kg and at or about 5.5×10⁵ cells/kg, between at or about 5.5×10⁵ cells/kg and at or about 6×10⁵ cells/kg, between at or about 6×10⁵ cells/kg and at or about 6.5×10⁵ cells/kg, between at or about 6.5×10⁵ cells/kg and at or about 7×10⁵ cells/kg, between at or about 7×10⁵ cells/kg and at or about 7.5×10⁵ cells/kg, between at or about 7.5×10⁵ cells/kg and at or about 8×10⁵ cells/kg, or between at or about 8×10⁵ of the cells/kg and at or about 10×10⁵ of the cells/kg. In some embodiments, the dose of cells comprises no more than 2×10⁵ of the CD19-directed CAR engineered cells per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 0.1×10⁵ of the CD19-directed CAR engineered cells per kilogram body weight of the subject (cells/kg), such as at least or at least about or at or about 0.2×10⁵ cells/kg, at least or at least about or at or about 0.3×10⁵ cells/kg, at least or at least about or at or about 0.4×10⁵ cells/kg, at least or at least about or at or about 0.5×10⁵ cells/kg, at least or at least about or at or about 0.6×10⁵ cells/kg, at least or at least about or at or about 0.7×10⁵ cells/kg, at least or at least about or at or about 0.8×10⁵ cells/kg, at least or at least about or at or about 0.9×10⁵ cells/kg, at least or at least about or at or about 0.1×10⁶ cells/kg, or at least or at least about or at or about 0.2×10⁶ cells/kg. In some embodiments, the number of cells is the number of such cells that are viable cells. In some embodiments, the number of cells is the number of such cells that are viable cells expressing the CD19-directed CAR, i.e., viable CAR+ cells.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or total cells in the composition administered. In some embodiments, the number of cells is the number of such cells that are viable cells. In some embodiments, the number of cells is the number of such cells that are viable cells expressing the CD19-directred CAR, i.e., viable CAR+ cells.

In some embodiments, the dose of cells is a flat dose of cells or fixed dose of cells such that the dose of cells is not tied to or based on the body surface area or weight of a subject. In some embodiments, administration of a higher number of cytotoxic cells based on weight of a subject may contribute to increased risk of toxicity, such as neurotoxicity, in the subject.

In some embodiments, the dose of genetically engineered cells comprises from at or about 1×10⁵ to at or about 1×10⁸ total T cells expressing the CD19-directed CAR, from at or about 1×10⁵ to at or about 1.0×10⁷ total T cells expressing the CD19-directed CAR, from at or about 1×10⁵ to at or about 1.0×10⁶ total T cells expressing the CD19-directed CAR, from at or about 1×10⁶ to at or about 1.0×10⁸ total T cells expressing the CD19-directed CAR, from at or about 1×10⁶ to at or about 1.0×10⁷ total T cells expressing the CD19-directed CAR, from at or about 5×10⁶ to at or about 1.0×10⁸ total T cells expressing the CD19-directed CAR, from at or about 5×10⁶ to at or about 1.0×10⁷ total T cells expressing the CD19-directed CAR, from at or about 10×10⁶ to at or about 1.0×10⁸ total T cells expressing the CD19-directed CAR. In some embodiments, the number of cells is the number of such cells that are viable cells, such as viable T cells.

In some embodiments, the dose of genetically engineered cells comprises from at or about 1×10⁵ to at or about 1×10⁸ total viable T cells expressing the CD19-directed CAR, from at or about 1×10⁵ to at or about 1.0×10⁷ total viable T cells expressing the CD19-directed CAR, from at or about 1×10⁵ to at or about 1.0×10⁶ total viable T cells expressing the CD19-directed CAR, from at or about 1×10⁶ to at or about 1.0×10⁸ total viable T cells expressing the CD19-directed CAR, from at or about 1×10⁶ to at or about 1.0×10⁷ total viable T cells expressing the CD19-directed CAR, from at or about 5×10⁶ to at or about 1.0×10⁸ total viable T cells expressing the CD19-directed CAR, from at or about 5×10⁶ to at or about 1.0×10⁷ total viable T cells expressing the CD19-directed CAR, from at or about 10×10⁶ to at or about 1.0×10⁸ total viable T cells expressing the CD19-directed CAR.

In some embodiments, the dose of cells is a relatively low dose. In some embodiments, anti-CD19 CAR T cell compositions for use in the provided embodiments include cells with a less differentiated phenotype, with a majority of the cells having a naive-like or central memory cell phenotype. Furthermore, in provided embodiments, compositions include populations of T cells in which greater than 25% of the T cells (e.g., CD3+ T cells) express the CAR, such as greater than 30%, 35%, 40%, 45% or 50% of the T cells (e.g., CD3+ T cells) express the CAR. In some embodiments, compositions include populations of T cells in which greater than 50% of the T cells (e.g., CD3+ T cells) express the CAR, such as greater than 60%, greater than 70% or greater than 80% of the T cells composition express the CAR. Without wishing to be bound by theory, compositions with features as provided herein ensure the cells exhibit higher potency and greater capacity to persist in the subject, while minimizing or reducing potential toxicity of the CAR-expressing T cells. In some embodiments, anti-CD19 CAR T cells of the dose exhibit higher potency, persistency and/or less toxicity than cells of alternative compositions that include a higher percentage of cells that are more differentiated (e.g., have a higher percentage of effector T cells). In some embodiments, anti-CD19 CAR T cells of the dose exhibit higher potency, persistency and/or less toxicity than cells of alternative compositions that include a lower percentage of cells that express the CAR. In some embodiments, the dose of genetically engineered cells may be administered in an amount that is less than 10×10⁷ total T cells expressing the CD19-directed CAR.

In some embodiments, the dose of genetically engineered cells may be administered in an amount that is less than 9×10⁷ total T cells expressing the CD19-directed CAR. In some embodiments, the dose of genetically engineered cells may be administered in an amount that is less than 8×10⁷ total viable T cells expressing the CD19-directed CAR. In some embodiments, the dose of genetically engineered cells may be administered in an amount that is less than 7.5×10⁷ total viable T cells expressing the CD19-directed CAR. In some embodiments, the dose of genetically engineered cells may be administered in an amount that is less than 7.0×10⁷ total viable T cells expressing the CD19-directed CAR. In some embodiments, the dose of genetically engineered cells may be administered in an amount that is less than 6.0×10⁷ total viable T cells expressing the CD19-directed CAR. In some embodiments, the dose of genetically engineered cells is from at or about 1×10⁶ to at or about 50×10⁶ total viable T cells expressing the CD19-directed CAR, from at or about 1×10⁶ to at or about 40×10⁶ total viable T cells expressing the CD19-directed CAR, at or about 1×10⁶ to at or about 30×10⁶ total viable T cells expressing the CD19-directed CAR, at or about 1×10⁶ to at or about 20×10⁶ total viable T cells expressing the CD19-directed CAR, at or about 1×10⁶ to at or about 10×10⁶ total viable T cells expressing the CD19-directed CAR, at or about 1×10⁶ to at or about 5×10⁶ total viable T cells expressing the CD19-directed CAR, at or about 1×10⁶ to at or about 2.5×10⁶ total viable T cells expressing the CD19-directed CAR, from at or about 2.5×10⁶ to at or about 50×10⁶ total viable T cells expressing the CD19-directed CAR, from at or about 2.5×10⁶ to at or about 40×10⁶ total viable T cells expressing the CD19-directed CAR, at or about 2.5×10⁶ to at or about 30×10⁶ total viable T cells expressing the CD19-directed CAR, at or about 2.5×10⁶ to at or about 20×10⁶ total viable T cells expressing the CD19-directed CAR, at or about 2.5×10⁶ to at or about 10×10⁶ total viable T cells expressing the CD19-directed CAR, at or about 2.5×10⁶ to at or about 5×10⁶ total viable T cells expressing the CD19-directed CAR, from at or about 2.5×10⁶ to at or about 50×10⁶ total viable T cells expressing the CD19-directed CAR, from at or about 5×10⁶ to at or about 50×10⁶ total viable T cells expressing the CD19-directed CAR, from at or about 5×10⁶ to at or about 40×10⁶ total viable T cells expressing the CD19-directed CAR, at or about 5×10⁶ to at or about 30×10⁶ total viable T cells expressing the CD19-directed CAR, at or about 5×10⁶ to at or about 20×10⁶ total viable T cells expressing the CD19-directed CAR, at or about 5×10⁶ to at or about 10×10⁶ total viable T cells expressing the CD19-directed CAR, from at or about 10×10⁶ to at or about 50×10⁶ total viable T cells expressing the CD19-directed CAR, from at or about 10×10⁶ to at or about 40×10⁶ total viable T cells expressing the CD19-directed CAR, at or about 10×10⁶ to at or about 30×10⁶ total viable T cells expressing the CD19-directed CAR, at or about 10×10⁶ to at or about 20×10⁶ total viable T cells expressing the CD19-directed CAR, from at or about 20×10⁶ to at or about 50×10⁶ total viable T cells expressing the CD19-directed CAR, from at or about 20×10⁶ to at or about 40×10⁶ total viable T cells expressing the CD19-directed CAR, at or about 20×10⁶ to at or about 30×10⁶ total viable T cells expressing the CD19-directed CAR, from at or about 30×10⁶ to at or about 50×10⁶ total viable T cells expressing the CD19-directed CAR, from at or about 30×10⁶ to at or about 40×10⁶ total viable T cells expressing the CD19-directed CAR, or from at or about 40×10⁶ to at or about 50×10⁶ total viable T cells expressing the CD19-directed CAR.

In some embodiments, the dose of genetically engineered cells is from at or at or about 0.1×10⁶ total T cells expressing the CD19-directed CAR, at or about 0.2×10⁶ total viable T cells expressing the CD19-directed CAR, at or about 0.25×10⁶ total viable T cells expressing the CD19-directed CAR, at or about 0.5×10⁶ total viable T cells expressing the CD19-directed CAR, at or about 0.75×10⁶ total T cells expressing the CD19-directed CAR, at or about 2×10⁶ total viable T cells expressing the CD19-directed CAR, at or about 3×10⁶ total viable T cells expressing the CD19-directed CAR, at or about 4×10⁶ total viable T cells expressing the CD19-directed CAR, at or about 6×10⁶ total viable T cells expressing the CD19-directed CAR, at or about 7×10⁶ total viable T cells expressing the CD19-directed CAR, at or about 8×10⁶ total viable T cells expressing the CD19-directed CAR, at or about 9×10⁶ total viable T cells expressing the CD19-directed CAR, at or about 10×10⁶ total viable T cells expressing the CD19-directed CAR, at or about 11×10⁶ total viable T cells expressing the CD19-directed CAR, at or about 12×10⁶ total viable T cells expressing the CD19-directed CAR, at or about 13×10⁶ total viable T cells expressing the CD19-directed CAR, at or about 14×10⁶ total viable T cells expressing the CD19-directed CAR, at or about 15×10⁶ total viable T cells expressing the CD19-directed CAR, at or about 16×10⁶ total viable T cells expressing the CD19-directed CAR, at or about 17×10⁶ total viable T cells expressing the CD19-directed CAR, at or about 18×10⁶ total viable T cells expressing the CD19-directed CAR, at or about 19×10⁶ total viable T cells expressing the CD19-directed CAR, at or about 25×10⁶ total viable T cells expressing the CD19-directed CAR, at or about 35×10⁶ total viable T cells expressing the CD19-directed CAR, at or about 45×10⁶ total viable T cells expressing the CD19-directed CAR, at or about 60×10⁶ total viable T cells expressing the CD19-directed CAR, at or about 70×10⁶ total T cells expressing the CD19-directed CAR, at or about 75×10⁶ total T cells expressing the CD19-directed CAR, at or about 80×10⁶ total viable T cells expressing the CD19-directed CAR, at or about 90×10⁶, 100×10⁶ total viable T cells expressing the CD19-directed CAR.

In some embodiments, the dose of genetically engineered cells is from at or about 5×10⁶ to at or about 50×10⁶ total viable T cells expressing the CD19-directed CAR. In some embodiments, the dose of genetically engineered cells is from at or about 10×10⁶ to at or about 50×10⁶ total viable T cells expressing the CD19-directed CAR.

In some embodiments, the dose of genetically engineered cells is about 5×10⁶ total viable T cells expressing the CD19-directed CAR. In some embodiments, a single dose of about 5×10⁶ T cells expressing the CD19-directed CAR is administered to the subject.

In some embodiments, the dose of genetically engineered cells is about 10×10⁶ total viable T cells expressing the CD19-directed CAR. In some embodiments, a single dose of about 10×10⁶ T cells expressing the CD19-directed CAR is administered to the subject.

In some embodiments, the dose of genetically engineered cells is about 15×10⁶ total viable T cells expressing the CD19-directed CAR. In some embodiments, a single dose of about 15×10⁶ T cells expressing the CD19-directed CAR is administered to the subject.

In some embodiments, the dose of genetically engineered cells is about 20×10⁶ total viable T cells expressing the CD19-directed CAR. In some embodiments, a single dose of about 20×10⁶ T cells expressing the CD19-directed CAR is administered to the subject.

In some embodiments, the dose of genetically engineered cells is about 25×10⁶ total viable T cells expressing the CD19-directed CAR. In some embodiments, a single dose of about 25×10⁶ T cells expressing the CD19-directed CAR is administered to the subject.

In some embodiments, the dose of genetically engineered cells is about 30×10⁶ total viable T cells expressing the CD19-directed CAR. In some embodiments, a single dose of about 30×10⁶ T cells expressing the CD19-directed CAR is administered to the subject.

In some embodiments, the dose of genetically engineered cells is about 40×10⁶ total viable T cells expressing the CD19-directed CAR. In some embodiments, a single dose of about 40×10⁶ T cells expressing the CD19-directed CAR is administered to the subject.

In some embodiments, the dose of genetically engineered cells is about 50×10⁶ total viable T cells expressing the CD19-directed CAR. In some embodiments, a single dose of about 50×10⁶ T cells expressing the CD19-directed CAR is administered to the subject.

In some embodiments, the number is with reference to the total number of CD3⁺, CD8⁺, or CD4+ and CD8+, in some cases also recombinant receptor-expressing (e.g., CAR⁺) cells. In some embodiments, the number of cells is the number of such cells that are viable cells.

In some embodiments, the T cells of the dose include CD4⁺ T cells, CD8⁺ T cells or CD4⁺ and CD8⁺ T cells.

In some embodiments, the T cells of the dose include CD4⁺ T cells, CD8⁺ T cells or CD4⁺ and CD8⁺ T cells.

In some embodiments, the dose of cells, e.g., recombinant receptor-expressing T cells, is administered to the subject as a single dose or is administered only one time within a period of two weeks, one month, three months, six months, one year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

In particular embodiments, the numbers and/or concentrations of cells refer to the number of recombinant receptor (e.g., CAR)-expressing cells. In other embodiments, the numbers and/or concentrations of cells refer to the number or concentration of T cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the dose is determined based on one or more criteria such as response of the subject to prior treatment and/or likelihood or incidence of the subject developing toxic outcomes, e.g., CRS, macrophage activation syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g., consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells is generally large enough to be effective in reducing disease burden.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as at or about 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment and/or likelihood or incidence of the subject developing toxic outcomes, e.g., CRS, macrophage activation syndrome, neurotoxicity, and/or a host immune response against the cells and/or recombinant receptors being administered.

### II. CD19-Directed Chimeric Antigen Receptors and Engineered Cells

In some embodiments, the provided methods and uses relate to methods and uses for administering engineered cells (e.g., T cells) expressing a CD19-directed chimeric antigen receptor (CAR) that binds to CD19 on B cells of the autoimmune disease or condition. In provided embodiments, binding to CD19 by the CAR engineered cells results in a cytotoxic response against the cells that express CD19. In some embodiments, the CD19-directed CAR binds to CD19 and mediates cytokine production and/or cytotoxic activity against CD19+ target cells when expressed in a T cell and stimulated via the CAR, such as by binding to CD19. In some embodiments, the CAR includes an extracellular antigen binding domain specific to CD19 that is linked to one or more intracellular signaling components, in some aspects via linkers and/or transmembrane domain(s). In some embodiments, the CD19-directed CAR is a bispecific CAR that is also specific to another B cell antigen. In some aspects, the engineered cells are provided as pharmaceutical compositions and formulations suitable for administration to a subject, such as for adoptive cell therapy. Also provided are therapeutic methods for administering the cells and compositions to a subject, e.g., a patient.

### A. CD19-Directed CARs

In some embodiments, the CD19-directed CAR comprises an antigen-binding domain directed to CD19, a hinge spacer, a transmembrane domain, and an intracellular signaling domain containing a costimulatory signaling region and an activating domain that provides a primary activation signal. In some aspects, the hinger spacer is between the antigen-binding domain and the transmembrane domain. In some embodiments, the CD19-directed CAR comprises, in N- to C-terminal order, an antigen-binding domain directed to CD19, a hinge spacer, a transmembrane domain, and an intracellular signaling domain containing a costimulatory signaling region and an activating domain that provides a primary activation signal.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

In some embodiments, the antigen-binding domain is an antibody or antigen-binding fragment or portion that targets CD19. In some embodiments, the antigen-binding domain is a portion of an antibody molecule, generally a variable heavy (V_{H}) chain region and/or variable light (V_{L}) chain region of the antibody, e.g., an scFv antibody fragment. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb). In some embodiments, an antigen-binding fragment comprises antibody variable regions, V_{H} and V_{L}, joined by a flexible linker. In such embodiments, the antibody or an antigen-binding fragment (e.g., scFv) contains a variable heavy chain and a variable light chain with six CDRs, CDRH1-3 and CDRL1-3, that confer binding to CD19. In some embodiments, the antigen-binding domain is a single domain antibody (sdAb), such as sdFv, nanobody, V_{H}H and V_{NAR}. In some embodiments, a single domain antibody is a V_{H}H that contains three CDRs, CDRH1-3.

In some embodiments, the scFv contains a V_{H} and a V_{L} derived from an antibody or an antibody fragment specific to CD19. In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR may comprise or consist of the V_{H}, the V_{L}, and/or one or more CDRs of any of the antibodies. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, may be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 4 and 5, respectively, and CDR-H3 set forth in SEQ ID NO: 6 or 7 and CDR-L1 set forth in SEQ ID NO: 1 and CDR-L2 set forth in SEQ ID NO: 2 or 8 and CDR-L3 sequences set forth in SEQ ID NO: 3 or 9.

In some embodiments, the antigen-binding domain is an scFv that comprises a variable light chain containing the CDR-L1 sequence of SEQ ID NO:1, a CDR-L2 sequence of SEQ ID NO:2, and a CDR-L3 sequence of SEQ ID NO:3 and a variable heavy chain containing a CDR-H1 sequence of SEQ ID NO:4, a CDR-H2 sequence of SEQ ID NO:5, and a CDR-H3 sequence of SEQ ID NO:6, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the antigen-binding domain is an scFv that comprises a variable light chain containing the CDR-L1 sequence of SEQ ID NO:1, a CDR-L2 sequence of SEQ ID NO:8, and a CDR-L3 sequence of SEQ ID NO:9 and a variable heavy chain containing a CDR-H1 sequence of SEQ ID NO:4, a CDR-H2 sequence of SEQ ID NO:5, and a CDR-H3 sequence of SEQ ID NO:7, or a variant of any of the foregoing having at least 85%, 86%, 87%, 88%, 89%, 90%, 91%, 92%, 93%, 94%, 95%, 96%, 97%, 98%, 99% or more sequence identity thereto.

In some embodiments, the scFv comprises a variable heavy chain region of FMC63 set forth in SEQ ID NO:10 and a variable light chain region of FMC63 set forth in SEQ ID NO:11, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the FMC63 antibody comprises the heavy chain variable region (V_{H}) comprising the amino acid sequence of SEQ ID NO: 10 and the light chain variable region (V_{L}) comprising the amino acid sequence of SEQ ID NO: 11.

In some embodiments, the variable heavy and variable light chains are connected by a linker. In some embodiments, the linker is set forth in SEQ ID NO:12. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:13 or a sequence that exhibits at least 85%, 86%, 87%, 88%, 89%, 90%, 91%, 92%, 93%, 94%, 95%, 96%, 97%, 98%, or 99% sequence identity to SEQ ID NO:13. In some embodiments, the scFv comprises the sequence of amino acids set forth in SEQ ID NO:14 or a sequence that exhibits at least 85%, 86%, 87%, 88%, 89%, 90%, 91%, 92%, 93%, 94%, 95%, 96%, 97%, 98%, or 99% sequence identity to SEQ ID NO:14.

In some embodiments the antigen-binding domain includes a V_{H} and/or V_{L} derived from SJ25C1, which, in some aspects, may be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 18-20, respectively, and CDR-L1, CDR-L2 and CDR-L3 sequences set forth in SEQ ID NOS: 15-17, respectively. In some embodiments, the svFv comprises a variable light chain containing a CDR-L1 sequence of SEQ ID NO:15, a CDR-L2 sequence of SEQ ID NO: 16, and a CDR-L3 sequence of SEQ ID NO:17 and a variable heavy chain containing a CDR-H1 sequence of SEQ ID NO:18, a CDR-H2 sequence of SEQ ID NO:19, and a CDR-H3 sequence of SEQ ID NO:20, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:21 and a variable light chain region of SJ25C1 set forth in SEQ ID NO:22, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the SJ25C1 antibody comprises the heavy chain variable region (V_{H}) comprising the amino acid sequence of SEQ ID NO: 21 and the light chain variable region (V_{L}) comprising the amino acid sequence of SEQ ID NO: 22. In some embodiments, the variable heavy and variable light chains are connected by a linker. In some embodiments, the linker is set forth in SEQ ID NO:23. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:24 or a sequence that exhibits at least 85%, 86%, 87%, 88%, 89%, 90%, 91%, 92%, 93%, 94%, 95%, 96%, 97%, 98%, or 99% sequence identity to SEQ ID NO:24.

In some embodiments, the anti-CD19 CAR includes an antigen-binding domain described in PCT Pub. No. WO2015187528. In some embodiments, the anti-CD19 CAR is a CAR described in PCT Pub. No. WO2015187528.

In some embodiments, the anti-CD19 CAR includes an antigen-binding domain that is a single chain antibody derived from a fully human antibody. In some embodiments, the single chain antibody is an scFv. Exemplary fully human anti-CD19 antibodies are described in PCT Pub. No. WO2016033570, PCT Pub. No. WO2020233589, U.S. Pub. No. US2010/0104509 and U.S. Pub. No. US20220220200.

In some embodiments, the anti-CD19 CAR includes a spacer containing a hinge domain. In some embodiments, the hinge domain comprises or consists of the formula X1PPX₂P, where X₁ is glycine, cysteine or arginine and X₂ is cysteine or threonine, as set forth in SEQ ID NO: 28. The spacer may be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, Hudecek et al. (2015) Cancer Immunol Res. 3(2): 125-135 or international patent application publication number WO2014031687.

In some embodiments, the spacer in a CD8a hinge domain, for example, a human CD8a hinge domain. In some embodiments, the CD8a hinge domain comprises or consists of an amino acid sequence set forth in SEQ ID NO:25. In some embodiments, the hinge domain comprises a CD28 hinge domain, for example, a human CD28 hinge domain. In some embodiments, the CD28 hinge domain comprises or consists of an amino acid sequence set forth in SEQ ID NO:26. In some embodiments, the CD28 hinge domain comprises or consists of an amino acid sequence set forth in SEQ ID NO:27. In some embodiments, the hinge domain has a sequence of amino acids that has at least 80% sequence identity, such as at least 85%, at least 90%, at least 95%, at least 96%, at least 97%, at least 98%, at least 99%, or 100% sequence identity to any of the foregoing.

In some embodiments, the spacer includes an immunoglobulin hinge domain. In some embodiments, the spacer contains only a hinge region of an IgG, such as only a hinge of IgG4 or IgG1, such as the hinge only spacer set forth in SEQ ID NO: 29, and encoded by the sequence set forth in SEQ ID NO: 30. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 32. In some embodiments, the spacer the spacer is an Ig hinge, e.g., an IgG4 hinge, linked to a C_{H}3 domain only, such as set forth in SEQ ID NO: 31. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 33. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 29, 31, 32 and 33.

In some embodiments, the transmembrane domain of the CAR is linked or fused between the antigen binding domain (e.g., scFv) or optionally the hinge domain and the intracellular signaling domain. The antigen binding domain and transmembrane may be linked directly or indirectly. In some embodiments, the antigen binding domain and transmembrane are between a hinge domain spacer, such as any described herein. In some embodiments, a transmembrane domain that naturally is associated with one of the domains in the receptor, e.g., CAR, is used. In some embodiments, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 (4-1BB), or CD154. Alternatively, the transmembrane domain in some embodiments is synthetic. In some embodiments, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28 or a variant thereof.

In some embodiments, the transmembrane domain is a transmembrane domain of human CD28 or variant thereof, e.g., a 27-amino acid transmembrane domain of a human CD28 (Accession No.: P10747.1). In some embodiments, the transmembrane domain is a transmembrane domain that comprises the sequence of amino acids set forth in SEQ ID NO: 34 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:34. In some embodiments, the transmembrane-domain containing portion of the recombinant receptor comprises the sequence of amino acids set forth in SEQ ID NO: 35 or a sequence of amino acids having at least or at least about 85%, 86%, 87%, 88%, 89%, 90%, 91%, 92%, 93%, 94%, 95%, 96%, 97%, 98%, 99% or more sequence identity to SEQ ID NO: 35.

In some embodiments, the transmembrane domain of the is a transmembrane domain of a human CD8a. In some embodiments, the transmembrane domain is a transmembrane domain that comprises the sequence of amino acids set forth in SEQ ID NO: 36 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:36.

In some embodiments, the intracellular signaling domain of the CAR provides for activation of the cell (e.g., T cell) into which it is engineered when engaged or ligated with the antigen recognized by the antigen binding domain. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. In particular embodiments, the intracellular signaling region mimics or approximate a signal of a natural antigen receptor through the primary activation domain and a signal through such a receptor in combination with a costimulatory receptor through the costimulatory domain. In some embodiments, upon ligation of the CAR by antigen, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal.

In some embodiments, the primary activation domain of the intracellular signaling domain comprises an ITAM. In some embodiments, the primary activation domain regulates primary activation of the TCR complex. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta. In some embodiments, the CD3-zeta chain is a human CD3-zeta chain.

In some embodiments, the intracellular (or cytoplasmic) signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 37, 38 or 39 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 37, 38 or 39. In some embodiments, the CD3(signaling domain comprises or consists of an amino acid sequence set forth in SEQ ID NO:37. In some embodiments, the CD3(signaling domain comprises or consists of an amino acid sequence set forth in SEQ ID NO:38. In some embodiments, the CD3(signaling domain comprises or consists of an amino acid sequence set forth in SEQ ID NO:39.

In some embodiments, the intracellular domain also includes a costimulatory domain. In some embodiments, the costimulatory domain is an intracellular domain of a T cell costimulatory molecule. In some embodiments, the CAR includes a signaling domain and/or transmembrane portion of a costimulatory receptor, such as CD28, 4-1BB, OX40 (CD134), CD27, DAP10, DAP12, ICOS and/or other costimulatory receptors. In some embodiments, the CAR includes a costimulatory region or domain of CD28 or 4-1BB, such as of human CD28 or human 4-1BB. In some embodiments, the intracellular signaling region further comprises a CD28 and CD137 (4-1BB, TNFRSF9) co-stimulatory domains, linked to a CD3 zeta intracellular domain. In some embodiments, the CD28 is a human CD28. In some embodiments, the 4-1BB is a human 4-1BB.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain may comprise the sequence of amino acids set forth in SEQ ID NO: 40 or 41 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 40 or 41. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 42 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 42.

Exemplary antigen receptors, e.g., CARs, also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654).

Additional exemplary antigen receptors, e.g., CARs, also include the CARs of YTB323 (rapcabtagene autoleucel), MB-CART19.1 (Mougiakakos et al., CD19-targeted CAR T cells in refractory systemic lupus erythematosus. N Engl J Med 2021; 385:567-9), MB-CART2019.1 (zamtocabtagene autoleucel, see WO2019/028051), prizloncabtagene autoleucel (C-CAR038, JNJ-90014496, see WO2021/188681), CABA-101 and CABA-201 (Peng et al., Molecular Therapy Methods & Clinical Development, Volume 32, Issue 2, 101267), KYV-101 (U.S. Pat. No. 10,287,350B2, WO2023/133092), IMPT-514 (see the CAR described in Larson et al., Cancer Discovery. 2023; 13(3):580-597).

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an antibody such as an antibody fragment, including scFvs, e.g., specific for CD19 such as any described above, a spacer, such as a spacer containing a hinge region, such as an Ig-hinge containing spacer, a transmembrane domain containing all or a portion of a CD28-derived transmembrane domain, a CD28-derived intracellular signaling domain, and a CD3 zeta signaling domain.

In some embodiments, the CAR contains an antibody, e.g., antibody fragment, such as an scFv, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. For example, in some such embodiments, the CAR includes an antibody or fragment, such as scFv, a spacer such as a spacer containing a hinge region, such as any of the Ig-hinge containing spacers, a CD28-derived transmembrane domain, a 4-1BB-derived intracellular signaling domain, and a CD3 zeta-derived signaling domain.

In particular embodiments, the CAR is a CD19-directed CAR containing an scFv antigen-binding domain from FMC63; an immunoglobulin hinge spacer, a transmembrane domain, and an intracellular signaling domain containing a costimulatory signaling region that is a signaling domain of 4-1BB and a signaling domain of a CD3-zeta (CD3( ) chain. In some embodiments, the scFv ha a VL having CDRs having an amino acid sequences RASQDISKYLN (SEQ ID NO: 1), an amino acid sequence of SRLHSGV (SEQ ID NO: 2), and an amino acid sequence of GNTLPYTFG (SEQ ID NO: 3); and a V_{H} with CDRs having an amino acid sequence of DYGVS (SEQ ID NO: 4), an amino acid sequence of VIWGSETTYYNSALKS (SEQ ID NO: 5) and YAMDYWG (SEQ ID NO: 6). In some embodiments, the scFv contains the variable heavy sequence set forth in SEQ ID NO:10 and the variable light sequence set forth in SEQ ID NO: 11, joined by a linker. In some embodiments, the scFv contains the sequence set forth in SEQ ID NO:14. In some embodiments, the transmembrane domain has a sequence that has at least 85%, 86%, 87%, 88%, 89%, 90%, 91%, 92%, 93%, 94%, 95%, 96%, 97%, 98%, 99% or more sequence identity to SEQ ID NO:34. In some embodiments, the transmembrane domain has the sequence set forth in SEQ ID NO:34. In some embodiments, the 4-1BB costimulatory signaling domain has a sequence at least 85%, 86%, 87%, 88%, 89%, 90%, 91%, 92%, 93%, 94%, 95%, 96%, 97%, 98%, 99% or more sequence identity to SEQ ID NO:42. In some embodiments, the 4-1BB costimulatory signaling domain has the sequence set forth in SEQ ID NO:42. In some embodiments, the CD3zeta signaling domain has a sequence having at least 85%, 86%, 87%, 88%, 89%, 90%, 91%, 92%, 93%, 94%, 95%, 96%, 97%, 98%, 99% or more sequence identity to SEQ ID NO: 37. In some embodiments, the CD3-zeta domain has the sequence set forth in SEQ ID NO: 37. In some embodiments, the CAR contains a hinge-containing immunoglobulin spacer between the scFv and the transmembrane domain. In some embodiments, the spacer is set forth in SEQ ID NO:29.

In particular embodiments of any of the provided methods, the CAR contains in order from N-terminus to C-terminus: an extracellular antigen-binding domain that is the scFv set forth in SEQ ID NO: 14, the spacer set forth in SEQ ID NO:29, the transmembrane domain set forth in SEQ ID NO:34, the 4-1BB costimulatory signaling domain set forth in SEQ ID NO:42, and the signaling domain of a CD3-zeta (CD3( ) chain set forth in SEQ ID NO:37.

In some embodiments, the CAR has a sequence having at least 85%, 86%, 87%, 88%, 89%, 90%, 91%, 92%, 93%, 94%, 95%, 96%, 97%, 98%, 99% or more sequence identity to SEQ ID NO:43. In some embodiments, the CAR comprises the sequence set forth in SEQ ID NO:43. In some embodiments, the CAR is set forth in SEQ ID NO:43. In some embodiments, the CAR is the CD19 CAR as present in Lisocabtagene maraleucel.

In some embodiments, the CAR is encoded by a sequence of nucleotides having at least 85%, 86%, 87%, 88%, 89%, 90%, 91%, 92%, 93%, 94%, 95%, 96%, 97%, 98%, 99% or more sequence identity to SEQ ID NO:44. In some embodiments, the CAR is encoded by a sequence of nucleotides set forth in SEQ ID NO: 44.

In some embodiments, the CAR contains in order from N-terminus to C-terminus: an extracellular antigen-binding domain that is an scFv comprising a variable heavy chain region of FMC63 set forth in SEQ ID NO:10 and a variable light chain region of FMC63 set forth in SEQ ID NO:11, such as the scFv set forth in SEQ ID NO: 14, the CD8a hinge domain of SEQ ID NO:25, the CD8a transmembrane domain of SEQ ID NO:36, the 4-1BB costimulatory domain of SEQ ID NO:42, the CD3(signaling domain of SEQ ID NO:37. In some embodiments, the CAR has a sequence having at least 85%, 86%, 87%, 88%, 89%, 90%, 91%, 92%, 93%, 94%, 95%, 96%, 97%, 98%, 99% or more sequence identity to any of the foregoing sequences. In some embodiments, the CAR has a sequence of amino acids having at least 85%, 86%, 87%, 88%, 89%, 90%, 91%, 92%, 93%, 94%, 95%, 96%, 97%, 98%, 99% or more sequence identity to SEQ ID NO: 45. In some embodiments, the CAR has the sequence set forth in SEQ ID NO: 45. In some embodiments, the CAR is the CD19 CAR as present in Tisagenlecleucel.

In some embodiments, the CAR contains in order from N-terminus to C-terminus: an extracellular antigen-binding domain that is an scFv comprising a variable heavy chain region of FMC63 set forth in SEQ ID NO:10 and a variable light chain region of FMC63 set forth in SEQ ID NO:11, such as the scFv set forth in SEQ ID NO: 14, the CD28 hinge domain of SEQ ID NO:26, the CD28 transmembrane domain of SEQ ID NO:34 or 35, the CD28 costimulatory domain of SEQ ID NO:40, the CD3(signaling domain of SEQ ID NO:37. In some embodiments, the CAR has a sequence of amino acids having at least 85%, 86%, 87%, 88%, 89%, 90%, 91%, 92%, 93%, 94%, 95%, 96%, 97%, 98%, 99% or more sequence identity to SEQ ID NO: 46. In some embodiments, the CAR has the sequence set forth in SEQ ID NO: 46. In some embodiments, the CAR is the CD19 CAR as present in Axicabtagene ciloleucel.

In some embodiments, the CAR contains an extracellular binding domain composed of an scFv derived from the anti-CD19 antibody known as Hu19. In some embodiments, the CAR contains the scFv derived from Hu19, a CD8a hinge and transmembrane domain (e.g., SEQ ID NO:47), a CD28 costimulatory domain (e.g., SEQ ID NO: 40) and a CD3(signaling domain (e.g., SEQ ID NO: 37). In some embodiments, the scFv designated Hu19 contains a light chain variable region (SEQ ID NO: 48), a linker peptide (GSTSGSGKPGSGEGSTKG [SEQ ID NO: 49]), and a heavy chain variable region (SEQ ID NO: 50). The scFv also may include a human CD8a leader sequence (SEQ ID NO: 51). In some embodiments, the CAR has the sequence set forth in SEQ ID NO:52. In some embodiments, the CAR has the sequence set forth in SEQ ID NO:53. In some embodiments, the CAR contains the scFv derived from Hu19, a CD8a hinge and transmembrane domain, a 4-1iB costimulatory domain and a CD3(signaling domain. In some embodiments, the CAR has the sequence set forth in SEQ ID NO:54. In some embodiments, the CAR does not include a signal sequence.

In some embodiments, the CAR contains an extracellular binding domain composed of an scFv derived from a fully human antibody, and an intracellular signaling domain comprising a 4-1BB costimulatory domain and a CD3(signaling domain. In some embodiments, the light chain variable region of the scFv comprises an amino acid sequence set forth in SEQ ID NO: 55, and the heavy chain variable region of the scFv Comprises an amino acid sequence set forth in SEQ ID NO: 56. In some embodiments, the light chain variable region of the scFv comprises an amino acid sequence set forth in SEQ ID NO: 57, and the heavy chain variable region of the scFv comprises an amino acid sequence set forth in SEQ I) NO: 58, 1 some embodiment, t sCFv has the sequence set forth in SEQ ID NO:59. In some embodiments, the scFv has the sequence set forth in SEQ ID NO: 60.

In some embodiments, the CAR contains a fully human anti-CD19 antibody, a CD8a hinge and transmembrane domains, a CD28 costimulatory domain and a CD3(activation domain. In some embodiments, the CAR has the sequence set forth in SEQ ID NO:61 or a sequence that has at least 85%, at least 90%, at least 95% or at least 98% sequence identity to SEQ ID NO:61. In some embodiments, the CAR has the sequence set forth in SEQ ID NO:62 or a sequence that has at least 85%, at least 90%, at least 95% or at least 98% sequence identity to SEQ ID NO:62. In some embodiments, the CAR has the sequence set forth in SEQ ID NO:63 or a sequence that has at least 85%, at least 90%, at least 95% or at least 98% sequence identity to SEQ ID NO:63. In some embodiments, the CAR has the sequence set forth in SEQ ID NO:64 or a sequence that has at least 85%, at least 90%, at least 95% or at least 98% sequence identity to SEQ ID NO:64. In some embodiments, the CAR has the sequence set forth in SEQ ID NO:65 or a sequence that has at least 85%, at least 90%, at least 95% or at least 98% sequence identity to SEQ ID NO:65. In some embodiments, the CAR has the sequence set forth in SEQ ID NO:66 or a sequence that has at least 85%, at least 90%, at least 95% or at least 98% sequence identity to SEQ ID NO:66. In some embodiments, the CAR has the sequence set forth in SEQ ID NO:67 or a sequence that has at least 85%, at least 90%, at least 95% or at least 98% sequence identity to SEQ ID NO:67. In some embodiments, the CAR has the sequence set forth in SEQ ID NO:68 or a sequence that has at least 85%, at least 90%, at least 95% or at least 98% sequence identity to SEQ ID NO:68. In some embodiments, the CAR has the sequence set forth in SEQ ID NO:69 or a sequence that has at least 85%, at least 90%, at least 95% or at least 98% sequence identity to SEQ ID NO:69. In some embodiments, the CAR has the sequence set forth in SEQ ID NO:70 or a sequence that has at least 85%, at least 90%, at least 95% or at least 98% sequence identity to SEQ ID NO:70. In some embodiments, the CAR has the sequence set forth in SEQ ID NO:71 or a sequence that has at least 85%, at least 90%, at least 95% or at least 98% sequence identity to SEQ ID NO:71. In some embodiments, the CAR has the sequence set forth in SEQ ID NO:72 or a sequence that has at least 85%, at least 90%, at least 95% or at least 98% sequence identity to SEQ ID NO:72. In some embodiments, the CAR has the sequence set forth in SEQ ID NO:73 or a sequence that has at least 85%, at least 90%, at least 95% or at least 98% sequence identity to SEQ ID NO:73. In some embodiments, the CAR is a CAR described in U.S. patent Ser. No. 10/287,350, e.g., Table 1 therein. In some embodiments, the CAR is a Hu19-CD828Z (KYV-101) which has a scFv from a fully-human anti-CD19 monoclonal antibody, CD8a hinge and transmembrane domains, a CD28 costimulatory domain and a CD3(activation domain.

In some embodiments, the CAR targets CD19 and at least one other antigen expressed on B cells. In some embodiments, the antigen associated with the disease or disorder is selected from CD20, CD19, CD22, ROR1, BCMA, CD45, CD21, CD5, CD33, Igkappa, Iglambda, CD79a, CD79b or CD30. In some embodiments, the other antigen is CD20 and the CAR is a CD20/CD19 directed CAR product. In some embodiments, the CAR is a bispecific CAR in which the extracellular antigen-binding domain binds CD19 and the one other antigen (e.g. CD20). In some embodiments, the bispecific CAR is a tandem CAR containing a first antigen binding domain that binds CD19 and a second antigen binding domain that binds the other antigen (e.g. CD20). In some embodiments, the CD19 directed scFv comprises a variable heavy chain region and a variable light chain region of FMC63 (e.g. variable heavy chain region set forth in SEQ ID NO:10 and a variable light chain region set forth in SEQ ID NO:11). In some embodiments, the CD19 scFv is Hu19 and comprises the variable heavy chain region set forth in SEQ ID NO:50 and the variable light chain region set forth in SEQ ID NO:48. In some embodiments, the first and second antigen binding domain may be positioned in any order, in which one antigen binding domain is distal and the other is proximal to the spacer and transmembrane domain. In some embodiments, each antigen binding domain comprises a variable heavy (V_{H}) chain and a variable light (VL) chain for targeting the antigen. In some embodiments, the V_{H} chain is N-terminal to the VL chain of the scFv. In some embodiments, the VL chain is C-terminal to the VL chain of the scFv. In some embodiments, each antigen binding domain is an scFv. In some embodiments, each antigen binding domain is a single domain antibody, such as a V_{H}H. Exemplary dual binding CARs are known, such as described in PCT publ. No. WO2019/028051 and Zhu et al. (2018) Cytotherapy, 20:394-406.

In some embodiments, the second antigen binding domain is directed against CD20. In some embodiments, the CD20 directed scFv comprises a variable heavy chain region and a variable light chain region of Leu16 (e.g. variable heavy chain region set forth in SEQ ID NO:74 and a variable light chain region set forth in SEQ ID NO:75). In some embodiments, the CD20 directed scFv comprises a variable heavy chain region and a variable light chain region of Ofatumumab (e.g., variable heavy chain region set forth in SEQ ID NO:76 and a variable light chain region set forth in SEQ ID NO:77). In some embodiments, the antigen binding domain is an scFv derived from a CD20 antibody described in U.S. patent publ. No. US2021/0363245. In some embodiments, the antigen binding domain is an scFv derived from the CD20 antibody C2B8 (e.g., described in U.S. Pat. No. 5,736,137), 11B8 (e.g., described in U.S patent application 2004/0167319), 8G6-5 (e.g, described in U.S. patent application 2009/0035322), 2.1.2 (e.g., described in WO 2006/130458), or GA101 (e.g., described in U.S. Pat. No. 9,539,251).

In some embodiments, the CAR is a CD19/CD20 tandem CAR. In some embodiments, the CAR contains an extracellular antigen binding domain composed of a CD19 scFv in tandem with a scFv from the Leu16 antibody specific for CD20; a CD8-derived spacer hinge and transmembrane region, a 4-1BB costimulatory domain and a CD3zeta signaling domain. In some embodiments, the CD20 scFv is distal and the CD19 scFv is proximal to the hinge and transmembrane region. In some embodiments, the CAR is set forth in SEQ ID NO:81. In some embodiments, the nucleotide sequence encoding the CAR is the set forth in SEQ ID NO:80. In some embodiments, the CAR is set forth in SEQ ID NO:83. In some embodiments, the nucleotide sequence encoding the CAR is set forth in SEQ ID NO:82. In some embodiments, the CAR does not include the leader sequence. In some embodiments, the CD20/CD19 CAR is the CD20/CD19 CAR present in zamtocabtagene autoleucel.

In some embodiments, the CAR is a CD19/CD20 tandem bispecific CAR. In some embodiments, the CAR contains an extracellular antigen binding domain composed of an anti-CD19 scFv from the FMC63 antibody specific to CD19, an anti-CD20 scFv from the Leu16 antibody specific for CD20; a CD8-derived spacer hinge and transmembrane region, a 4-1BB costimulatory domain and a and a CD3zeta signaling domain.

In some embodiments, the CAR is a CD19/CD20 tandem CAR containing an extracellular antigen binding domain composed of a CD19 scFv in tandem with a scFv from the Leu16 antibody specific for CD20; a CD8-derived spacer hinge and transmembrane region, a 4-1BB costimulatory domain and a CD3zeta signaling domain. In some embodiments, the CD19 scFv contains the V_{H} sequence set forth in SEQ ID NO:10 and the VL chain region set forth in SEQ ID NO:11. In some embodiments, the V_{H} is N-terminal to the VL in the CD19 scFv. In some embodiments the V_{H} is C-terminal to the VL in the CD19 scFv. In some embodiments, the CD20 scFv contains the V_{H} sequence set forth in SEQ ID NO:74 and the VL chain region set forth in SEQ ID NO:75. In some embodiments, the V_{H} is N-terminal to the VL in the CD20 scFv. In some embodiments the V_{H} is C-terminal to the VL in the CD20 scFv. In some embodiments, the CD20 scFv is distal and the CD19 scFv is proximal to the hinge and transmembrane region, such that the CAR as a tandem link of the anti-CD20 scFv followed by the FMC63 scFv. In some embodiments, the CAR has the sequence set forth in SEQ ID NO: 84 or a sequence that has at least 85%, 90%, 95% or 98% sequence identity to SEQ ID NO: 84. In some embodiments, the CD20/CD19 CAR is TN-LEU-19, for example as described in WO2021/188681.

In some embodiments, the CAR is a CD19/CD20 tandem CAR containing an extracellular antigen binding domain composed of a CD19 scFv in tandem with a scFv from the Ofatumumab antibody specific for CD20; a CD8-derived spacer hinge and transmembrane region, a 4-1BB costimulatory domain and a CD3zeta signaling domain. In some embodiments, the CD19 scFv contains the V_{H} sequence set forth in SEQ ID NO:10 and the VL chain region set forth in SEQ ID NO:11. In some embodiments, the V_{H} is N-terminal to the VL in the CD19 scFv. In some embodiments the V_{H} is C-terminal to the VL in the CD19 scFv. In some embodiments, the CD20 scFv contains the V_{H} sequence set forth in SEQ ID NO:76 and the VL chain region set forth in SEQ ID NO:77. In some embodiments, the V_{H} is N-terminal to the VL in the CD20 scFv. In some embodiments the V_{H} is C-terminal to the VL in the CD20 scFv. In some embodiments, the CD20 scFv is distal and the CD19 scFv is proximal to the hinge and transmembrane region, such that the CAR as a tandem link of the anti-CD20 scFv followed by the FMC63 scFv. In some embodiments, the CAR has the sequence set forth in SEQ ID NO: 85 or a sequence that has at least 85%, 90%, 95% or 98% sequence identity to SEQ ID NO: 85. In some embodiments, the CD20/CD19 CAR is TN-OF-19 (C-CAR039), for example as described in WO2021/188681.

In some embodiments, the second antigen binding domain is directed against BCMA. In some embodiments, the second antigen binding domain is an scFv derived from a BCMA directed antibody, such as any described herein. In some embodiments, the antigen binding domain is an scFv targeting BCMA that comprises the heavy chain variable region shown in SEQ ID NO: 78, and an antibody light chain variable region shown in SEQ ID NO: 79.

In some embodiments, the CAR is a CD19/BCMA tandem CAR. In some embodiments, the CAR contains an extracellular antigen binding domain targeting CD19 and BCMA, in which the antigen binding domain (scFv) targeting CD19 in the bispecific CAR comprises an antibody heavy chain variable region shown in SEQ ID NO: 10 and an antibody light chain variable region shown in SEQ ID NO: 11, and the antigen binding domain (scFv) targeting BCMA in the bispecific CAR comprises an antibody heavy chain variable region shown in SEQ ID NO: 78 and an antibody light chain variable region shown in SEQ ID NO: 79. In some embodiments, the bispecific CAR for targeting CD19 and BCMA antigens includes in order the anti-CD19 scFv, the anti-BCMA scFv, a hinge region (e.g. CD8 hinge), a transmembrane region, and an intracellular T cell signal region including a costimulatory signaling domain (e.g. CD28 domain or 4-1BB domain) and a CD3zeta signaling domain. In some embodiments, the CD19scFv and BCMAscFv are connected by a short peptide segment (G4S)xN. In some embodiments, the CAR is a CAR as described in US2022/0202864.

### B. Nucleic Acids

In some embodiments, the cells, e.g., T cells, are genetically engineered to express a recombinant receptor. In some embodiments, the engineering is carried out by introducing polynucleotides that encode the CAR. In some embodiments, the nucleic acid sequence encoding the CAR contains a signal sequence that encodes a signal peptide. In some aspects, the signal sequence may encode a signal peptide derived from a native polypeptide. In other aspects, the signal sequence may encode a heterologous or non-native signal peptide. Also provided are polynucleotides encoding the CAR, and vectors or constructs containing such nucleic acids and/or polynucleotides.

In some embodiments, the nucleic acid sequence encoding the CAR contains a signal sequence that encodes a signal peptide. In some aspects, the signal sequence may encode a signal peptide derived from a native polypeptide. Non-limiting exemplary examples of signal peptides include, for example, the GMCSFR alpha chain signal peptide set forth in SEQ ID NO: 86 and encoded by the nucleotide sequence set forth in SEQ ID NO:87, or the CD8 alpha signal peptide set forth in SEQ ID NO:88.

### C. Cells and Methods of Engineering Cells

In some embodiments, engineered cells, such as those that express an anti-CD19 CAR as described, used in accord with the provided methods and uses are produced or generated by exemplary processes as described in, for example, PCT/US2019/046062, WO 2019/089855 and WO 2015/164675.

The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample may be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, immune cells to be engineered, such as T cells, are selected, isolated or enriched from the sample. In some embodiments, the method includes immunoaffinity-based selection of T cells. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some aspects of such processes, a volume of cells is mixed with an amount of a desired affinity-based selection reagent. The immunoaffinity-based selection may be carried out using any system or method that results in a favorable energetic interaction between the cells being separated and the molecule specifically binding to the marker on the cell, e.g., the antibody or other binding partner on the solid surface, e.g., particle. Non-limiting methods for cell selection include, for example, magnetic bead-based separation methods, chromatography-based methods and flow cytometry by fluorescence-activated cell sorting (FACs). In some embodiments, methods are carried out using particles such as beads, e.g., magnetic beads, that are coated with a selection agent (e.g., antibody) specific to the marker of the cells. The particles (e.g., beads) may be incubated or mixed with cells in a container, such as a tube or bag, while shaking or mixing, with a constant cell density-to-particle (e.g., bead) ratio to aid in promoting energetically favored interactions.

In some aspects, the separation and/or other steps are carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

In certain embodiments, separation and/or other steps are carried out using the CliniMACS Prodigy system (Miltenyi Biotec). The CliniMACS Prodigy® system in some aspects is equipped with a cell processing unit that permits automated washing and fractionation of cells by centrifugation. The CliniMACS Prodigy® system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy® system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a biological sample, e.g., a sample of PBMCs or other white blood cells, is subjected to selection of T cells. In some embodiments, the selection results in an enriched composition of input cells in which at least 75%, 80%, 85%, 90%, 95%, 96%, 97%, 98%, or 99% of the cells in the composition are T cells. In some embodiments, a biological sample, e.g., a sample of PBMCs or other white blood cells, are subjected to selection of CD3+ T cells. In some embodiments, the selection results in an enriched composition of input cells in which at least 75%, 80%, 85%, 90%, 95%, 96%, 97%, 98%, or 99% of the cells in the composition are CD3+ T cells. In some embodiments, a biological sample, e.g., a sample of PBMCs or other white blood cells, are subjected to selection of CD4+ T cells and CD8+ T cells. In some embodiments, the selection results in an enriched composition of input cells in which at least 75%, 80%, 85%, 90%, 95%, 96%, 97%, 98%, or 99% of the cells in the composition are CD4+ and CD8+ T cells.

In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In particular embodiments, the T cells are activated or stimulated by contacting the cells with T cell stimulatory reagent. T cell stimulating reagents include one or more agents, such as antibodies, that are able to engage the TCR to initiate a primary signal in the cells and engage a costimulatory receptor signal. In some embodiments, the agents include an agent specific for a TCR, e.g., anti-CD3, and also an agent for stimulating a costimulatory receptor, e.g., anti-CD28. In some embodiments, the T cell stimulatory reagents include an anti-CD3 antibody or antigen binding fragment (e.g., Fab) and an anti-CD28 antibody or antigen binding fragment (e.g., Fab). In some embodiments, such agents may be, bound to solid support such as a bead. In particular embodiments, the one or more agents comprise a streptavidin-based oligomer, such as a streptavidin mutein oligomer, conjugated to Strep-tagged anti-CD3 and Strep-tagged anti-CD28 Fabs. In some embodiments, the oligomeric particle reagent is any as described in WO2015/158868 or WO2018/197949. Among the stimulatory reagents are anti-CD3/anti-CD28 beads (e.g., DYNABEADS® M-450 CD3/CD28 T Cell Expander, Detachable Dynabeads™, ExpACT® beads, or EXPAMER™). In some embodiments, the stimulation further includes culture of the cells with one or more cytokines. In some embodiments, the cytokine includes one or more of IL-2, IL-15 and/or IL-7. In particular embodiments, the one or more cytokines are recombinant cytokines. In some embodiments, the one or more cytokines are human recombinant cytokines. In some embodiments, the stimulating conditions include temperature suitable for the growth of human T lymphocytes, for example, at least about 25 degrees Celsius, generally at least about 30 degrees Celsius, and generally at or about 37 degrees Celsius. In some embodiments, the incubation is performed in serum free media.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy, 2014 Apr 3. doi: 10.1038/gt.2014.25; Carlens et al. (2000) Exp. Hematol., 28(10): 1137-46; Alonso-Camino et al. (2013) Mol. Ther. Nucl. Acids., 2, e93; Park et al., Trends Biotechnol., 2011 November 29(11): 550-557). Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In particular embodiments, genetic engineering, such as by transforming (e.g., transducing) the cells with a viral vector, further includes one or more steps of incubating the cells after the introducing or contacting of the cells with the viral vector.

In some such embodiments, the further incubation is effected under conditions to result in integration of the viral vector into a host genome of one or more of the cells. For example, the further incubation provides time for the viral vector that may be bound to the T cells following transduction, e.g., via spinoculation, to integrate within the genome of the cell to delivery the gene of interest. In some aspects, the further incubation is carried out under conditions to allow the cells, e.g., transformed cells, to rest or recover in which the culture of the cells during the incubation supports or maintains the health of the cells. In particular embodiments, the cells are incubated under static conditions, such as conditions that do not involve centrifugation, shaking, rotating, rocking, or perfusion, e.g., continuous or semi-continuous perfusion of the media. In some embodiments, the incubation includes culture of the cells with one or more cytokines. In some embodiments, the cytokine includes one or more of IL-2, IL-15 and/or IL-7. In particular embodiments, the one or more cytokines are recombinant cytokines. In some embodiments, the one or more cytokines are human recombinant cytokines. In some embodiments, the incubation is for between about 1 day and about 6 days, such as between about 2 days and about 4 days.

In some embodiments, the methods of engineering the cells produce a cell therapy for treating the subject. Thus, in some embodiments, the methods provide for a therapeutically effective dose for treating the subject. In some embodiments, the engineered cell therapy is autologous to the subject. In some embodiments, cells of the cell therapy are harvested after the incubation and are formulated for administration to a subject. In some embodiments, the cells are formulated in a pharmaceutically acceptable buffer, which may, in some aspects, include a pharmaceutically acceptable carrier or excipient. In some embodiments, the cells are formulated with a cryoprotectant (e.g., DMSO), such as 5% to 10% DMSO solution, e.g., about 7.5% DMSO. In some embodiments, the cells are further formulated with human serum albumin (HSA) at a concentration between about 0.1% w/v and about 4% w/v, such as about 0.1% and about 1% w/v.

### D. Compositions and Formulations

The provided methods and uses involve use or administration of a dose of engineered cells of a composition comprising engineered T cells expressing an anti-CD19 CAR such as a CAR targeting human CD19. In certain embodiments, the composition is a therapeutic composition enriched in T cells, e.g., composition enriched in CD3+ T cells or composition enriched in CD4+ and CD8+ T cells, manufactured using a process for generating or producing the engineered cell therapy comprising CAR engineered T cells disclosed herein, e.g., in Section II-C.

In some embodiments, the methods involve washing the transduced and/or incubated cells to replace the cells in a pharmaceutically acceptable buffer that may include one or more optional pharmaceutically acceptable carriers or excipients. Exemplary of such pharmaceutical forms, including pharmaceutically acceptable carriers or excipients, may be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

Pharmaceutically acceptable carriers will generally be sterile, at least for human use. A pharmaceutical composition will generally comprise agents for buffering and preservation in storage, and may include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

The pharmaceutical composition in some embodiments contains agents or cells in amounts effective to treat or prevent the disease or condition, such as a therapeutically effective dose. Compositions may be formulated for administration to a subject. In some embodiments, the cell populations are administered to a subject using peripheral systemic delivery by intravenous, intraperitoneal, or subcutaneous injection.

In some embodiments, at least 30%, at least 40%, at least 45%, at least 50%, at least 55%, at least 60%, at least 65%, at least 70%, at least 75%, at least 80%, at least 85%, or at least 90%, at least 95%, of the cells of cell therapy composition express the CAR. In certain embodiments, at least 50% of the cells of the cell therapy composition express the CAR. In certain embodiments, at least 30%, at least 40%, at least 45%, at least 50%, at least 55%, at least 60%, at least 65%, at least 70%, at least 75%, at least 80%, at least 85%, at least 90%, or at least 95%, of the CD3+ T cells of the cell therapy composition express the CAR.

In particular embodiments, the composition comprising engineered T cells expressing an anti-CD19 CAR is enriched in CD4+ and CD8+ T cells. In particular embodiments, CD3+ CD4+cells account for between about 55% and about 65% of the total live CD45+ cells in the composition, while CD3+ CD8+ cells account for between about 35% and about 45% of the total live CD45+ cells in the composition. In particular embodiments, CD3+ CD4+ cells account for about 60% while CD3+ CD8+ cells account for about 40% of the total live CD45+ cells in the composition. In particular embodiments, the composition contains a ratio of between 5:1 and 1:5, between 3:1 and 1:3, between 2.5:1 and 1:2.5, between 2:1 and 1:2, between 1.5:1 and 1:1.5, between 1.4:1 and 1:1.4, between 1.3:1 and 1:1.3, between 1.2:1 and 1:1.2, or between 1.1:1 and 1:1.1 CD4+ T cells to CD8+ T cells. In some embodiments, the composition of cells has a ratio of or of about 5:1, of our about 3:1, of or of about 2.8:1, of or of about 2.5:1, of or of about 2.25:1, of or of about 2:1, of or of about 1.8:1, of or of about 1.7:1, of or of about 1.6:1, of or of about 1.5:1, of or of about 1.4:1, of or of about 1.3:1, of or of about 1.2:1, of or of about 1.1:1, of or of about 1:1, of or of about 1:1.1, of or of about 1:1.2, of or of about 1:1.3, of or of about 1:1.4, of or of about 1:1.5, of or of about 1:1.6, of or of about 1:1.7, of or of about 1:1.8, of or of about 1:2, of or of about 1:2.25, of or of about 1:2.5, of or of about 1:2.8, of or of about 1:3, or of or of about 1:5 CD4+ T cells to CD8+ T cells. In some embodiments, the ratio is the number of such T cells that express the CAR.

In particular embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable T cells. In some embodiments, the composition contains at least at or about 75% viable T cells. In certain embodiments, the composition contains at least at or about 85%, at least at or about 90%, or at least at or about 95% viable T cells. In some embodiments, at least 70% of the T cells in the composition are viable T cells. In some embodiments, at least 75% of the T cells in the composition are viable T cells. In some embodiments, at least 80% of the T cells in the composition are viable T cells. In some embodiments, at least 85% of the T cells in the composition are viable T cells. In some embodiments, at least 90% of the T cells in the composition are viable T cells. In some embodiments, the T cells are characterized as CD3+. In some of any embodiments, viability is determined by staining for acridine orange (AO) and propidium iodide (PI).

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

The terms “polypeptide” and “protein” are used interchangeably to refer to a polymer of amino acid residues and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as severe refractory SLE). This delay may be of varying lengths of time, depending on the history of the disease and/or individual being treated. In some embodiments, sufficient or significant delay can, in effect, encompass prevention, in that the individual does not develop the disease.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress or reduce immune activity compared to the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### IV. Exemplary Embodiments

Among the provided embodiments are:
1. A method of treating a subject having Myasthenia gravis (MG), the method comprising administering a dose of CD19-directed genetically modified T cells to a subject having or suspected of having Myasthenia gravis (MG), wherein the T cells of the dose are positive for expression of a chimeric antigen receptor (CAR) that binds CD19 and the dose is from 1×106 to 50×106 CAR-positive, optionally viable, T cells, wherein the subject has severe disease that is refractory to three or more prior therapies for treating MG.
2. A method for reducing Myasthenia gravis (MG) disease activity, the method comprising administering a dose of CD19-directed genetically modified T cells to a subject having or suspected of having Myasthenia gravis (MG), wherein the T cells of the dose are positive for expression of a chimeric antigen receptor (CAR) that binds CD19 and the dose is from 1×106 to 50×106 CAR-positive, optionally viable, T cells, wherein the subject has severe disease that is refractory to three or more prior therapies for treating MG.
3. The method of embodiment 1 or embodiment 2, wherein the prior therapy is eculizumab, efgartigimod, or Rystiggo.
4. The method of any one of embodiments 1-3, wherein the MG is anti-AChR antibody positive.
5. The method of any one of embodiments 1-4, wherein the MG is anti-MuSK antibody positive.
6. The method of any one of embodiments 1-5, wherein the MG is anti-LRP4 positive.
7. A method of treating a subject having primary Sjogren's Disease (SjD), the method comprising administering a dose of CD19-directed genetically modified T cells to a subject having or suspected of having primary Sjogren's Disease (SjD), wherein the T cells of the dose are positive for expression of a chimeric antigen receptor (CAR) that binds CD19 and the dose is from 1×106 to 50×106 CAR-positive, optionally viable, T cells, wherein the subject is characterized with refractory disease that is refractory to at least one prior therapy for treating SjD and/or with extraglandular disease.
8. A method for reducing primary Sjogren's Disease (SjD) disease activity, the method comprising administering a dose of CD19-directed genetically modified T cells to a subject having or suspected of having primary Sjogren's Disease (SjD), wherein the T cells of the dose are positive for expression of a chimeric antigen receptor (CAR) that binds CD19 and the dose is from 1×106 to 50×106 CAR-positive, optionally viable, T cells, wherein the subject is characterized with refractory disease that is refractory to at least one prior therapy for treating SjD and/or with extraglandular disease.
9. The method of embodiment 7 or embodiment 8, wherein the subject has refractory disease to at least one prior therapy.
10. The method of embodiment 9, wherein the disease is a glandular disease.
11. The method of embodiment 9, wherein the disease is an extraglandular disease.
12. The method of any one of embodiments 9-11, wherein at least one prior therapy is a hydroxychloroquine, oral glucocorticoid, immunosuppressive agent or an anti-CD20 antibody, optionally wherein the anti-CD20 antibody is Rituximab.
13. The method of any one of embodiments 7-9, wherein the subject has extraglandular disease.
14. The method of embodiment 13, wherein the subject has severe extraglandular disease is characterized by one or more of cutaneous vasculitis, renal involvement, peripheral nervous system (PNS) involvement, interstitial lung disease (ILD), or cryoglobulinemia.
15. The method of any one of embodiments 7-14, wherein the subject has anti-Sjogren's syndrome A (SSA) antibodies or anti-Sjogren's syndrome B (SSB) antibodies.
16. The method of any one of embodiments 7-14, wherein the subject has high disease activity as determined by the European League Against Rheumatism (EULAR) Sjogren Syndrome Disease Activity Index (ESSDAI) score of 14 or higher, optionally characterized by multiple organ involvement.
17. A method of treating a subject having ANCA-associated vasculitis (AAV), the method comprising administering a dose of CD19-directed genetically modified T cells to a subject having or suspected of having ANCA-associated vasculitis (AAV), wherein the T cells of the dose are positive for expression of a chimeric antigen receptor (CAR) that binds CD19 and the dose is from 1×106 to 50×106 CAR-positive, optionally viable, T cells.
18. A method for reducing ANCA-associated vasculitis (AAV), the method comprising administering a dose of CD19-directed genetically modified T cells to a subject having or suspected of having ANCA-associated vasculitis (AAV), wherein the T cells of the dose are positive for expression of a chimeric antigen receptor (CAR) that binds CD19 and the dose is from 1×106 to 50×106 CAR-positive, optionally viable, T cells.
19. The method of embodiment 17 or embodiment 18, wherein the subject has a Birmingham Vasculitis Activity Score (BVAS) greater than 16.
20. The method of any of embodiments 17-19, wherein the subject has relapsed following remission after treatment with a prior therapy, optionally wherein the relapse is within 5 years of receiving the prior therapy.
21. The method of embodiment 20, wherein the prior therapy is a corticosteroid, optionally a high-dose glucocorticoid, an anti-CD20 antibody, optionally rituximab, a complement inhibitor or an intravenous immunoglobulin.
22. The method of any of embodiments 17-21, wherein the subject is treatment refractory, optionally wherein the subject fails treatment with an immunosuppressant or disease-modifying antirheumatic drug (DMARDs) and intravenous immunoglobulin.
23. The method of any one of embodiments 17-22, wherein the subject is cytoplasmic-ANCA or proteinase-3-ANCA positive.
24. The method of any one of embodiments 17-23, wherein the subject is characterized by severe disease with organ involvement or organ damage, optionally wherein the organ is renal or pulmonary.
25. A method for post-induction maintenance therapy for treating ANCA-associated vasculitis (AAV), the method comprising administering a dose of CD19-directed genetically modified T cells to a subject having received an induction therapy for treating ANCA-associated vasculitis (AAV), wherein the T cells of the dose are positive for expression of a chimeric antigen receptor (CAR) that binds CD19 and the dose is from 1×106 to 50×106 CAR-positive, optionally viable, T cells.
26. The method of embodiment 25, wherein the induction therapy comprises one or more of cyclophosphamide, an anti-CD20 antibody (e.g., rituximab), avacophan, methotrexate or mycophenolate mofetil.
27. The method of embodiment 25, wherein the induction therapy comprises an anti-CD20 antibody, optionally rituximab, in combination with a corticosteroid, optionally a glucocorticoid.
28. A method of treating a subject having Rheumatoid Arthritis (RA), the method comprising administering a dose of CD19-directed genetically modified T cells to a subject having or suspected of having Rheumatoid Arthritis (RA), wherein the T cells of the dose are positive for expression of a chimeric antigen receptor (CAR) that binds CD19 and the dose is from 1×106 to 50×106 CAR-positive, optionally viable, T cells, wherein the subject has severe disease that is refractory to three or more prior therapies for treating RA.
29. A method for reducing Rheumatoid Arthritis (RA) disease activity, the method comprising administering a dose of CD19-directed genetically modified T cells to a subject having or suspected of having Rheumatoid Arthritis (RA), wherein the T cells of the dose are positive for expression of a chimeric antigen receptor (CAR) that binds CD19 and the dose is from 1×106 to 50×106 CAR-positive, optionally viable, T cells, wherein the subject has severe disease that is refractory to three or more prior therapies for treating RA.
30. The method of embodiment 28 or embodiment 29, wherein the at least two of the three or more prior therapies are at least two previous treatments with a disease-modifying anti-rheumatic drug (DMARD).
31. The method of embodiment 22 or embodiment 30, wherein the DMARD is methotrexate, sulfasalazine, hydroxychloroquine, and leflunomide; a TNF antagonist (e.g., adalimumab, etanercept or infliximab); or a targeted synthetic DMARD (e.g., JAK inhibitor, such as baricitinib and tofacitinib).
32. The method of any of embodiments 28-31, wherein the subject has severe extra-articular disease, optionally characterized by one or more of renal involvement, peripheral nervous system (PNS) involvement, interstitial lung disease (ILD).
33. The method of any of embodiments 28-32, wherein the subject has a high DAS28 score, optionally a score of greater than 5.1.
34. The method of any of embodiments 28-33, wherein the subject is positive for rheumatoid factor (RF) and anti-cyclic citrullinated peptide antibody (ACPA).
35. The method of any of embodiments 28-34, wherein the subject has disease characterized by severe organ involvement that includes renal involvement, peripheral nervous system (PNS) involvement, or interstitial lung disease (ILD).
36. A method of treating a subject having Autoimmune Encephalitis (AE), the method comprising administering a dose of CD19-directed genetically modified T cells to a subject having or suspected of having Autoimmune Encephalitis (AE), wherein the T cells of the dose are positive for expression of a chimeric antigen receptor (CAR) that binds CD19 and the dose is from 1×106 to 50×106 CAR-positive, optionally viable, T cells.
37. A method for reducing Autoimmune Encephalitis (AE) disease activity, the method comprising administering a dose of CD19-directed genetically modified T cells to a subject having or suspected of having Autoimmune Encephalitis (AE), wherein the T cells of the dose are positive for expression of a chimeric antigen receptor (CAR) that binds CD19 and the dose is from 1×106 to 50×106 CAR-positive, optionally viable, T cells.
38. The method of embodiment 36 or embodiment 37, wherein the subject has severe disease that is refractory to one or more prior therapies.
39. The method of embodiment 38, wherein the one or more prior therapy is two or more prior therapies.
40. The method of embodiment 38 or 39, wherein the one or more prior therapy is one or more of a high-dose steroid, intravenous immunoglobulins (IVIG), intravenous methylprednisolone (IVMP), plasma exchange (PLEX) or immunosuppressant.
41. The method of any of embodiments 36-40, wherein the subject has relapsed or is refractory to two prior lines of therapy, wherein the first line of therapy is a high-dose steroid, intravenous immunoglobulins (IVIG), intravenous methylprednisolone (IVMP), or a plasma exchange (PLEX) and the second line of therapy is an immunosuppressant.
42. The method of embodiment 40 or embodiment 41, wherein the immunosuppressant is an anti-CD20 antibody, optionally Rituximab; rituximab and tocilizumab; cyclophosphamide; mycophenolate mofetil; or azathioprine.
43. A method for post-induction maintenance therapy for treating Autoimmune Encephalitis (AE), the method comprising administering a dose of CD19-directed genetically modified T cells to a subject having received an induction therapy for treating Autoimmune Encephalitis (AE), wherein the T cells of the dose are positive for expression of a chimeric antigen receptor (CAR) that binds CD19 and the dose is from 1×106 to 50×106 CAR-positive, optionally viable, T cells.
44. The method of embodiment 43, wherein the induction therapy comprises an anti-CD20 antibody, optionally rituximab.
45. The method of embodiment 44, wherein the induction therapy comprises an anti-CD20 antibody, optionally rituximab, in combination with a corticosteroid, optionally a glucocorticoid.
46. The method of any of embodiments 36-45, wherein the subject has anti-NMDAR antibodies or anti-LGI1 antibodies.
47. A method of treating a subject having Pemphigus, the method comprising administering a dose of CD19-directed genetically modified T cells to a subject having or suspected of having Pemphigus, wherein the T cells of the dose are positive for expression of a chimeric antigen receptor (CAR) that binds CD19 and the dose is from 1×106 to 50×106 CAR-positive, optionally viable, IT cells.
48. A method for reducing Pemphigus disease activity, the method comprising administering a dose of CD19-directed genetically modified T cells to a subject having or suspected of having Pemphigus, wherein the T cells of the dose are positive for expression of a chimeric antigen receptor (CAR) that binds CD19 and the dose is from 1×106 to 50×106 CAR-positive, optionally viable, T cells.
49. The method of embodiment 47 or embodiment 48, wherein the subject has disease that has relapsed or is refractory to one or more prior therapies for treating Pemphigus.
50. The method of embodiment 49, wherein the one or more prior therapy is a corticosteroid, azathioprine, methotrexate, an anti-CD20 antibody, optionally rituximab, or efgartigimod.
51. A method of treating a subject having Membranous Nephropathy (MN), the method comprising administering a dose of CD19-directed genetically modified T cells to a subject having or suspected of having Membranous Nephropathy (MN), wherein the T cells of the dose are positive for expression of a chimeric antigen receptor (CAR) that binds CD19 and the dose is from 1×106 to 50×106 CAR-positive, optionally viable, T cells.
52. A method for reducing Membranous Nephropathy (MN) disease activity, the method comprising administering a dose of CD19-directed genetically modified T cells to a subject having or suspected of having Membranous Nephropathy (MN), wherein the T cells of the dose are positive for expression of a chimeric antigen receptor (CAR) that binds CD19 and the dose is from 1×106 to 50×106 CAR-positive, optionally viable, T cells.
53. A method of treating a subject having Immunoglobulin G4-related disease (IgG4-RD), the method comprising administering a dose of CD19-directed genetically modified T cells to a subject having or suspected of having Immunoglobulin G4-related disease (IgG4-RD), wherein the T cells of the dose are positive for expression of a chimeric antigen receptor (CAR) that binds CD19 and the dose is from 1×106 to 50×106 CAR-positive, optionally viable, T cells.
54. A method for reducing Immunoglobulin G4-related disease (IgG4-RD) disease activity, the method comprising administering a dose of CD19-directed genetically modified T cells to a subject having or suspected of having Immunoglobulin G4-related disease (IgG4-RD), wherein the T cells of the dose are positive for expression of a chimeric antigen receptor (CAR) that binds CD19 and the dose is from 1×106 to 50×106 CAR-positive, optionally viable, T cells.
55. A method of treating a subject having Neuromyelitis optica spectrum disorder (NMOSD), the method comprising administering a dose of CD19-directed genetically modified T cells to a subject having or suspected of having Neuromyelitis optica spectrum disorder (NMOSD), wherein the T cells of the dose are positive for expression of a chimeric antigen receptor (CAR) that binds CD19 and the dose is from 1×106 to 50×106 CAR-positive, optionally viable, T cells.
56. A method for reducing Neuromyelitis optica spectrum disorder (NMOSD) disease activity, the method comprising administering a dose of CD19-directed genetically modified T cells to a subject having or suspected of having Neuromyelitis optica spectrum disorder (NMOSD), wherein the T cells of the dose are positive for expression of a chimeric antigen receptor (CAR) that binds CD19 and the dose is from 1×106 to 50×106 CAR-positive, optionally viable, T cells.
57. A method of treating a subject having Stiff-person syndrome (SPS), the method comprising administering a dose of CD19-directed genetically modified T cells to a subject having or suspected of having Stiff-person syndrome (SPS), wherein the T cells of the dose are positive for expression of a chimeric antigen receptor (CAR) that binds CD19 and the dose is from 1×106 to 50×106 CAR-positive, optionally viable, T cells.
58. A method for reducing Stiff-person syndrome (SPS) disease activity, the method comprising administering a dose of CD19-directed genetically modified T cells to a subject having or suspected of having Stiff-person syndrome (SPS), wherein the T cells of the dose are positive for expression of a chimeric antigen receptor (CAR) that binds CD19 and the dose is from 1×106 to 50×106 CAR-positive, optionally viable, T cells.
59. A method of treating a subject having Irritable bowel disease (IBD), the method comprising administering a dose of CD19-directed genetically modified T cells to a subject having or suspected of having Irritable bowel disease (IBD), wherein the T cells of the dose are positive for expression of a chimeric antigen receptor (CAR) that binds CD19 and the dose is from 1×106 to 50×106 CAR-positive, optionally viable, T cells.
60. A method for reducing Irritable bowel disease (IBD) disease activity, the method comprising administering a dose of CD19-directed genetically modified T cells to a subject having or suspected of having Irritable bowel disease (IBD), wherein the T cells of the dose are positive for expression of a chimeric antigen receptor (CAR) that binds CD19 and the dose is from 1×106 to 50×106 CAR-positive, optionally viable, T cells.
61. A method of treating a subject having Thrombotic Thrombocytopenia Purpura (TTP), the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×106 to 50×106 CAR-positive, optionally viable, T cells.
62. A method for reducing Thrombotic Thrombocytopenia Purpura (TTP) disease activity, the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×106 to 50×106 CAR-positive, optionally viable, T cells.
63. A method of treating a subject having Autoimmune hemolytic anemia (AIHA), the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×106 to 50×106 CAR-positive, optionally viable, T cells.
64. A method for reducing Autoimmune hemolytic anemia (AIHA) disease activity, the method comprising administering a dose of CD19-directed genetically modified T cells to a subject having or suspected of having Thrombotic Thrombocytopenia Purpura (TTP), wherein the T cells of the dose are positive for expression of a chimeric antigen receptor (CAR) that binds CD19 and the dose is from 1×106 to 50×106 CAR-positive, optionally viable, T cells.
65. A method of treating a subject having Immune thrombocytopenia (ITP), the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×106 to 50×106 CAR-positive, optionally viable, T cells.
66. A method for reducing Immune thrombocytopenia (ITP) disease activity, the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×106 to 50×106 CAR-positive, optionally viable, T cells.
67. The method of embodiment 65 or embodiment 66, wherein the ITP is chronic ITP.
68. A method of treating a subject having chronic Immune thrombocytopenia (cITP), the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×106 to 50×106 CAR-positive, optionally viable, T cells.
69. A method for reducing chronic Immune thrombocytopenia (cITP) disease activity, the method comprising administering a dose of CD19-directed genetically modified T cells to a subject having or suspected of having Immune thrombocytopenia (ITP), wherein the T cells of the dose are positive for expression of a chimeric antigen receptor (CAR) that binds CD19 and the dose is from 1×106 to 50×106 CAR-positive, optionally viable, T cells.
70. A method of treating a subject having IgA nephropathy, the method comprising administering a dose of CD19-directed genetically modified T cells to a subject having or suspected of having IgA nephropathy, wherein the T cells of the dose are positive for expression of a chimeric antigen receptor (CAR) that binds CD19 and the dose is from 1×106 to 50×106 CAR-positive, optionally viable, T cells.
71. A method for reducing IgA nephropathy disease activity, the method comprising administering a dose of CD19-directed genetically modified T cells to a subject having or suspected of having IgA nephropathy, wherein the T cells of the dose are positive for expression of a chimeric antigen receptor (CAR) that binds CD19 and the dose is from 1×106 to 50×106 CAR-positive, optionally viable, T cells.
72. A method of treating a subject having bullous pemphigoid (BP), the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×106 to 50×106 CAR-positive, optionally viable, T cells.
73. A method for reducing bullous pemphigoid (BP) disease activity, the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×106 to 50×106 CAR-positive, optionally viable, T cells.
74. A method of treating a subject having ulcerative colitis (UC), the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×106 to 50×106 CAR-positive, optionally viable, T cells.
75. A method for reducing ulcerative colitis (UC) disease activity, the method comprising administering a dose of CD19-directed genetically modified T cells to a subject having or suspected of having bullous pemphigoid (BP), wherein the T cells of the dose are positive for expression of a chimeric antigen receptor (CAR) that binds CD19 and the dose is from 1×106 to 50×106 CAR-positive, optionally viable, T cells.
76. The method of any of embodiments 1-75, wherein the systemic autoimmune disease is a relapsed or refractory disease to one or more prior therapies for treating the disease.
77. The method of any of embodiments 1-76, wherein the one or more prior therapies are two or more prior therapies for treating the disease.
78. The method of any of embodiments 1-77, wherein at least one of the one or more prior therapies is an anti-CD20 antibody, optionally rituximab.
79. The method of any of embodiments 1-78, wherein the systemic autoimmune disease is a severe disease.
80. The method of any of embodiments 1-79, wherein the dose is at or about 1×10⁶ to 40×106 CAR-positive, optionally viable, T cells.
81. The method of any of embodiments 1-79, wherein the dose is at or about 1×10⁶ to 25×106 CAR-positive, optionally viable, T cells.
82. The method of any of embodiments 1-79, wherein the dose is at or about 5×106 CAR-positive, optionally viable, T cells.
83. The method of any of embodiments 1-79, wherein the dose is at or about 10×106 CAR-positive, optionally viable, T cells.
84. The method of any of embodiments 1-79, wherein the dose is at or about 25×106 CAR-positive, optionally viable, T cells.
85. The method of any of embodiments 1-79, wherein the dose is at or about 50×106 CAR-positive, optionally viable, T cells.
86. The method of any of embodiments 1-85, wherein the T cells are autologous to the subject.
87. The method of any of embodiments 1-85, further comprising obtaining a leukapheresis sample from the subject for manufacturing the composition comprising engineered T cells.
88. The method of any of embodiments 1-87, wherein prior to the administration, the subject has been preconditioned with a lymphodepleting therapy.
89. The method of any of embodiments 1-88, wherein the method further comprises, immediately prior to the administration of the dose of CD19-directed genetically modified T cells, administering a lymphodepleting therapy to the subject, wherein the lymphodepleting therapy comprises the administration of fludarabine and/or cyclophosphamide.
90. The method of any of embodiments 1-89, wherein the administration of the dose of CD19-directed genetically modified T cells and/or the lymphodepleting therapy is carried out via outpatient delivery.
91. The method of any of embodiments 88-90, wherein the lymphodepleting therapy comprises the administration of fludarabine at 30 mg/m² body surface area of the subject, daily, and cyclophosphamide at 300 mg/m² body surface area of the subject, daily, each for 3 days.
92. The method of any of embodiments 88-90, wherein the dose of CD19-directed genetically modified T cells is administered between at or about 48 hours and at or about 9 days, inclusive, after completion of the lymphodepleting therapy.
93. The method of any of embodiments 1-92, wherein the dose of CD19-directed genetically modified T cells is administered to the subject by intravenous infusion.
94. The method of any of embodiments 1-93, wherein the CAR comprises an extracellular antigen-binding domain that binds CD19, a transmembrane domain, and an intracellular signaling domain.
95. The method of embodiment 94, wherein the CAR comprises a hinge spacer between the extracellular antigen-binding domain and the transmembrane domain, optionally wherein the hinge spacer is an immunoglobulin hinge or a CD8a hinge.
96. The method of embodiment 94 or embodiment 95, wherein the extracellular antigen-binding domain is an FMC63 monoclonal antibody-derived single chain variable fragment (scFv).
97. The method of any of embodiments 94-96, wherein the extracellular antigen-binding domain comprises a variable heavy chain set forth in SEQ ID NO:10 and a variable light chain set forth in SEQ ID NO:11.
98. The method of embodiment 96 or embodiment 97, wherein the scFv is set forth as SEQ ID NO: 14.
99. The method of embodiment 94 or embodiment 95, wherein the extracellular antigen-binding domain is a Hu19 single chain variable fragment (scFv).
100. The method of any one of embodiments 94, 95, and 99, wherein the extracellular antigen-binding domain comprises a variable heavy chain set forth in SEQ ID NO:50 and a variable light chain set forth in SEQ ID NO:48.
101. The method of embodiment 99 or 100, wherein the extracellular antigen-binding domain comprises in order a variable light chain set forth in SEQ ID NO: 48, a linker peptide set forth in SEQ ID NO: 49, and a variable heavy chain set forth in SEQ ID NO: 50.
102. The method of any one of embodiments 1-101, wherein the CAR is a monospecific CAR directed to CD19.
103. The method of any of embodiments 1-101, wherein the CAR is a tandem bispecific CAR directed against CD19 and at least one other antigen expressed on B cells.
104. The method of embodiment 103, wherein the other antigen expressed on B cells is selected from the group consisting of CD20, CD19, CD22, ROR1, BCMA, CD45, CD21, CD5, CD33, Igkappa, Iglambda, CD79a, CD79b or CD30.
105. The method of embodiment 103 or embodiment 104, wherein the other antigen expressed on B cells is CD20.
106. The method of embodiment 105, wherein the extracellular antigen-binding domain comprises a variable heavy chain and a variable light chain derived from a CD20 antibody selected from the group consisting of Leu16, C2B8, 11B8, 8G6-5, 2.1.2 and GA101
107. The method of any of embodiments 94-106, wherein the transmembrane domain is a CD28 transmembrane domain.
108. The method of any of embodiments 94-107, wherein the transmembrane domain is a transmembrane domain from CD28, optionally a transmembrane domain that comprises the sequence of amino acids set forth in SEQ ID NO: 34 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO:34.
109. The method of any of embodiments 105-108, wherein the intracellular signaling domain comprises a 4-1BB costimulatory domain and a CD3zeta activation domain.
110. The method of any of embodiments 1-109, wherein the CAR comprises, in order from N- to C-terminus, an FMC63 monoclonal antibody-derived single chain variable fragment (scFv), IgG4 hinge region, a CD28 transmembrane domain, a 4-1BB (CD137) costimulatory domain, and a CD3 zeta signaling domain.
111. The method of embodiment 109 or embodiment 110, wherein the 4-1BB costimulatory domain is or comprises the sequence set forth in SEQ ID NO: 42 or a variant thereof having at least 85%, 86%, 87%, 88%, 89%, 90%, 91%, 92%, 93%, 94%, 95%, 96%, 97%, 98%, 99% or more sequence identity to SEQ ID NO:42.
112. The method of any of embodiments 109-111, wherein the CD3zeta signaling domain is or comprises the sequence set forth in SEQ ID NO: 37, 38 or 39 or a sequence having at least 85%, 86%, 87%, 88%, 89%, 90%, 91%, 92%, 93%, 94%, 95%, 96%, 97%, 98%, 99% or more sequence identity thereto.
113. The method of any of embodiments 1-112, wherein the CAR contains in order from N-terminus to C-terminus: an extracellular antigen-binding domain that is the scFv set forth in SEQ ID NO: 14, the spacer set forth in SEQ ID NO:29, the transmembrane domain set forth in SEQ ID NO:34, the 4-1BB costimulatory signaling domain set forth in SEQ ID NO:42, and the signaling domain of a CD3-zeta (CD3( ) chain set forth in SEQ ID NO:37.
114. The method of any of embodiments 1-113, wherein the CAR comprises the amino acid sequence set forth in SEQ ID NO:43 or a sequence having at least 85%, 86%, 87%, 88%, 89%, 90%, 91%, 92%, 93%, 94%, 95%, 96%, 97%, 98%, 99% or more sequence identity to SEQ ID NO: 43
115. The method of any of embodiments 1-114, wherein the dose of T cells comprises CD4+ T cells expressing the CAR and CD8+ T cells expressing the CAR.
116. The method of any of embodiments 1-115, wherein the dose of T cells comprises CD4+ T cells expressing the CAR and CD8+ T cells expressing the CAR at a ratio between about 1:5 and about 5:1, optionally at a ratio between about 1:3 and about 3:1.
117. The method of any of embodiments 1-116, wherein at least or at least about 90% of the cells in the composition are CD3+ cells.
118. The method of any of embodiments 1-117, wherein at least or at least about 91%, at least or at least about 92%, at least or at least about 93%, at least or at least about 94%, at least or at least about 95%, or at least or at least about 96% of the cells in the composition are CD3+ cells.
119. The method of any of embodiments 1-118, wherein at least 25% of the T cells in the composition are CAR+ T cells
120. The method of any of embodiments 1-119, wherein at least 30%, at least 35%, at least 40%, at least 45% or at least 50% of the T cells in the composition are CAR+ T cells
121. The method of any of embodiments 1-120, wherein at least 80% of the T cells in the composition are viable T cells, optionally wherein viability is determined by staining for acridine orange (AO) and propidium iodide (PI).
122. The method of any of embodiments 1-121, wherein the subject does not receive administration of immunosuppressant for treating the disease after administering the dose of CD19-directed genetically modified T cells.
123. The method of any of embodiments 1-122, wherein the subject is human.

### V. EXAMPLES

The following examples are included for illustrative purposes only and are not intended to limit the scope of the invention.

### Example 1: Anti-CD19 CAR T cell Therapeutic Composition

Engineered compositions of primary T cells containing T cells expressing an anti-CD19 chimeric antigen receptor (CAR) are produced from patients with an autoimmune disease to be treated. The CAR contained an anti-CD19 scFv (e.g., SEQ ID NO:14), an immunoglobulin-derived spacer (e.g., SEQ ID NO:29), a transmembrane domain derived from CD28 (e.g., SEQ ID NO:34), a costimulatory region derived from 4-1BB (e.g., SEQ ID NO:42), and a CD3-zeta intracellular signaling domain (e.g., SEQ ID NO:37). A sequence of an exemplary anti-CD19 CAR used in this study is set forth in SEQ ID NO: 43. A viral vector containing the anti-CD19 CAR transgene is used for transduction of primary T cells. T are isolated from autologous subjects to be treated by immunoaffinity-based enrichment of CD4+ and CD8+ T cells from leukapheresis samples from the individual patients to be treated. The selected CD4+ and CD8+ T cell compositions are stimulated by incubation with anti-CD3/anti-CD28 stimulation reagent in a serum-free complete media containing recombinant IL-2 (100 IU/mL), recombinant IL-7 (600 IU/mL) and recombinant IL-15 (100 IU/mL). After stimulation, cells are transduced with a viral vector encoding an anti-CD19 CAR (e.g., SEQ ID NO:43) and are further incubated to achieve a dose for administration, and then are formulated with a cryoprotectant. The CD4+ and CD8+cryopreserved cell compositions are thawed prior to intravenous administration to the subject.

### Example 2: Immune Reset Observed in Patients Treated with CAR T Cells from Non-Expanded Process

An anti-CD19 CAR T cell drug product composition engineered to contain the anti-CD19 CAR as described in Example 1 was used to treat human patients with relapsed or refractory (R/R) non-Hodgkin's lymphoma (NHL). Following leukapheresis, patient T cells were purified and engineered followed by limited ex vivo expansion. The CAR contained an anti-CD19 scFv (e.g., SEQ ID NO:14), an immunoglobulin-derived spacer (e.g., SEQ ID NO:29), a transmembrane domain derived from CD28 (e.g., SEQ ID NO:34), a costimulatory region derived from 4-1BB (e.g., SEQ ID NO:42), and a CD3-zeta intracellular signaling domain (e.g., SEQ ID NO:37). A sequence of an exemplary anti-CD19 CAR used in this study is set forth in SEQ ID NO: 43. A viral vector containing the anti-CD19 CAR transgene were used to transduce primary T cells that were isolated from autologous subjects to be treated by immunoaffinity-based enrichment of CD4+ and CD8+ T cells from leukapheresis samples from the individual patients to be treated. The selected CD4+ and CD8+ T cell compositions were stimulated by incubation with anti-CD3/anti-CD28 stimulation reagent in a serum-free complete media containing recombinant IL-2 (100 IU/mL), recombinant IL-7 (600 IU/mL) and recombinant IL-15 (100 IU/mL). After stimulation, cells were transduced with the viral vector encoding the anti-CD19 CAR (e.g., SEQ ID NO:43) and were further incubated to achieve a dose for administration, and then were formulated with a cryoprotectant. The CD4+ and CD8+cryopreserved cell compositions were thawed prior to intravenous administration to the subject.

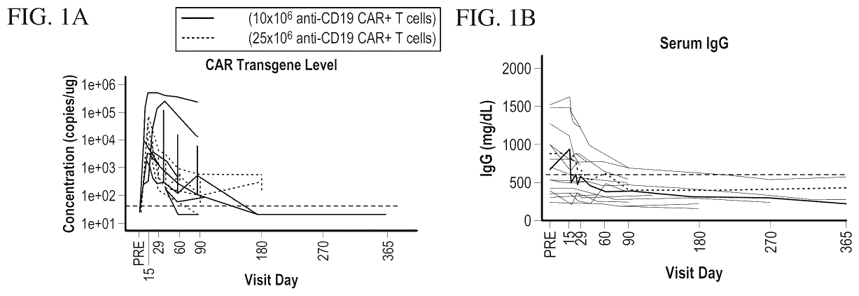

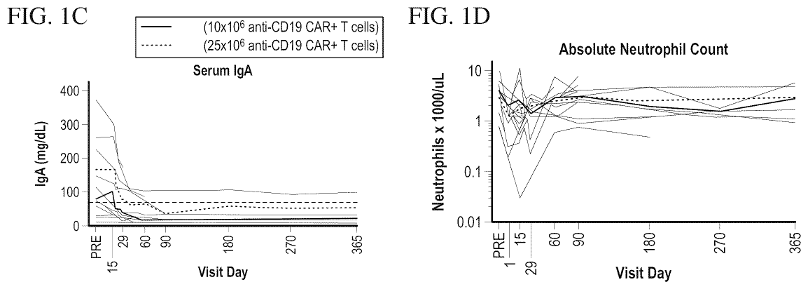

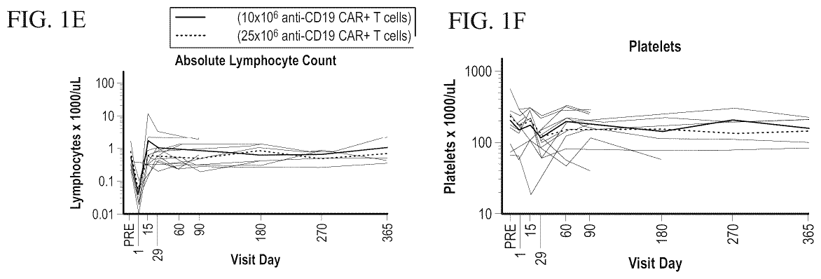

Patients received a single infusion of either 10×10⁶ or 25×10⁶ cells of the anti-CD19 CAR T cell drug product after lymphodepleting chemotherapy, which constituted 3 days of fludarabine at 30 mg/m² and cyclophosphamide at 300 mg/m². Transgene levels were measured in blood using droplet digital polymerase chain reaction and plotted using RStudio and is shown in FIG. 1A. The steady dotted line represents the limit of quantification which was 40 copies/ug. There was an increase in the transgene levels initially showing cellular expansion of the anti-CD19 CAR T cells in both doses of CAR T cells. FIG. 1B shows serum IgG and FIG. 1C shows serum IgA, with both showing a decrease in circulating immunoglobulin levels after administration of both doses CAR T cells. The steady dotted lines on FIG. 1B and FIG. 1C represent low normal immunoglobulin levels. Neutrophils, shown in FIG. 1D, lymphocytes, shown in FIG. 1E, and platelets, shown in FIG. 1F, all show a decrease in circulating cell levels with rebounds in cell levels by around 60 days post infusion with CAR T cells, demonstrating that the decrease in cell levels are transient and patients rapidly recover. This data demonstrates that hypogammaglobulinemia occurs indicating an immune reset (i.e., the suppression of overactive B cells by the anti-CD19 CAR T cells with subsequent restoration of homeostatic immune system function) in subjects treated with the anti-CD19 CAR T cells. These results, including the observed hypogammaglobulinemia, support the use of the anti-CD19 CAR for treating systemic autoimmune diseases that have a B cell involvement, and clinical remission may be observed at similar doses in patients with autoimmune diseases such as those as described.

### Example 3 Administration of Anti-CD19 CAR-Expressing Cells in Myasthenia Gravis (MG)

Therapeutic CAR+ T cell compositions containing autologous T cells expressing a chimeric antigen-receptor (CAR) specific for CD19 are administered to subjects with Myasthenia Gravis (MG). Subjects selected to be treated are subjects diagnosed with MG that have severe disease and/or have relapsed or are refractory to one, two, three or more prior therapies. Specifically, a cohort of subjects treated include subjects that have relapsed to three or more prior therapies (4L+), including subjects that have not achieved any response to a current therapy. Among the treated subjects are subjects that have a high Myasthenia Gravis Activities of Daily Living scale (MG-ADL) indicative of greater severity of symptoms. In some embodiments, the subjects are positive for anti-ChR or anti-MuSK antibodies. See, e.g., Muppidi, Srikanth et al. “Utilization of MG-ADL in myasthenia gravis clinical research and care.” Muscle & nerve vol. 65,6 (2022): 630-639.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×106 CAR-expressing viable T cells to 50×106 CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including assessment of treatment-emergent or serious adverse events (AEs), AEs of special interest, laboratory and/or imaging abnormalities, and Dose Limiting Toxicities (DLT).

Subjects are assessed for a reduction in symptoms including using anti-AChR Ab test, repetitive nerve stimulation (RNS) test and single-fiber electromyography (SFEMG) to assess conduction delays in the NMJ, edrophonium (Tensilon) test that increases the availability of ACh in the NMJ, ice-pack test, imaging (e.g., Chest computed tomography (CT) or magnetic resonance imaging (MRI)), and other laboratory tests.

Secondary endpoints related to efficacy and pharmacokinetics (PK) are also assessed, including assessment of MG-activities of daily living (ADL) score. An improvement in the MG-ADL score, such as at least a 2-point or 3-point improvement, indicates a clinical response to the anti-CD19 CAR T cell therapy.

### Example 4 Administration of Anti-CD19 CAR-Expressing Cells in Primary Sioren's Disease (SiD)

Therapeutic CAR+ T cell compositions containing autologous T cells expressing a chimeric antigen-receptor (CAR) specific for CD19 are administered to subjects with relapsing forms of Primary Sjogren's Disease (SjD).

Subjects with SjD including subjects with extraglandular manifestations, glandular manifestations, autoantibodies against SSA/Ro, autoantibodies against SSA/La, and/or with no response to current therapies are treated. Further, subjects with SjD as classified by the ACR-EULAR Classification Criteria are also treated. Eligibility criteria include subjects of all sexes being 18 years to 65 years old.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by using a Schirmer's test, (without anesthesia; ≤5 mm/5 minutes) and/or a vital dye staining of the eye surface, histopathology of a lip biopsy to identify focal lymphocytic sialoadenitis (focus score≥1 per 4 mm2), quantification of unstimulated whole salivary flow (≤1.5 mL in 15 minutes), a parotid sialography, a salivary scintigraphy, quantification of autoantibodies anti-SSA (Ro), anti-SSB (La), or both, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Disease activity indices developed for SjD and validated by the EULAR SjOgren's Task Force are also used as endpoint assessments: EULAR SS Patient Reported Index (ESSPRI) and EULAR SS Disease Activity Index (ESSDAI). Disease activity is also measured by a disease activity score using the Sjogren's Syndrome Symptom Diary (SSSD). Patient reported outcomes (PROs) are measured in the subject before and after administration of the CD19-targeted cell therapy. Change in status categories of improved, unchanged, worse, exacerbation, and died of SjD may be assessed and recorded after treatment of selected subjects with anti-CD19 CAR T cell therapy.

### Example 5 Administration of Anti-CD19 CAR-Expressing Cells in Anti-Neutrophil Cytoplasmic Antibody (ANCA)-Associated Vasculitides (AAVs)

Therapeutic CAR+ T cell compositions containing autologous T cells expressing a chimeric antigen-receptor (CAR) specific for CD19 are administered to subjects with relapsing forms of anti-neutrophil cytoplasmic antibody (ANCA)-associated vasculitides (AAVs.

Subjects with AAV including subjects with graniulomatosis with polyangiitis (GPA), microscopic polyangiitis (MPA), and eosinophilic GPA (EGPA), Proteinase 3 (PR3)-ANCA, and/or with no response to current therapies are treated. Further, subjects with severe and/or relapsed AAV are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×106 CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission using Birmingham Vasculitis Activity Score (BVAS) to assess the activity and severity of AAV, quantification of PR3, change from baseline in kidney function as measured by eGFR (estimated glomerular filtration rate), and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of AAV may be assessed and recorded after treatment of selected subjects with anti-CD19 CAR T cell therapy.

### Example 6 Administration of Anti-CD19 CAR-Expressing Cells in Rheumatoid Arthritis (RA)

Therapeutic CAR+ T cell compositions containing autologous T cells expressing a chimeric antigen-receptor (CAR) specific for CD19 are administered to subjects with relapsing forms rheumatoid arthritis (RA).

Subjects with RA including subjects positive for rheumatoid factor (RF) and/or anti-cyclic citrullinated peptide (CCP) IgG antibodies and/or with no response to current therapies are treated. Further, subjects with severe and/or relapsed RA are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission using criteria for classifying RA set forth by the American College of Rheumatology (ACR) to assess the activity and severity of RA, quantification of RF and/or CCP IgG antibodies, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of RA may be assessed and recorded after treatment of selected subjects with anti-CD19 CAR T cell therapy.

### Example 7 Administration of Anti-CD19 CAR-Expressing Cells in Autoimmune Encephalitis (AE)

Therapeutic CAR+ T cell compositions containing autologous T cells expressing a chimeric antigen-receptor (CAR) specific for CD19 are administered to subjects with relapsing forms of autoimmune encephalitis (AE).

Subjects with AE including subjects with severe and refractory AE and/or with no response to current therapies are treated. Subjects positive for LGI1 may be treated. Subjects having received an induction therapy (e.g., rituximab) may also be treated as a post-induction therapy. Further, subjects with severe and/or relapsed AE treated with one or more prior lines of treatment (e.g., intravenous methylprednisolone, intravenous immunoglobulin, plasma exchange) are also treated. Subjects with relapsed AE treated with three or more prior lines of treatment (e.g., tocilizumab, resiniferatoxin) are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of AE, quantification of LGI1 and/or NMDAR antibodies and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of AE may be assessed and recorded after treatment of selected subjects with anti-CD19 CAR T cell therapy.

### Example 8 Administration of Anti-CD19 CAR-Expressing Cells in Pemphigus

Therapeutic CAR+ T cell compositions containing autologous T cells expressing a chimeric antigen-receptor (CAR) specific for CD19 are administered to subjects with relapsing forms of pemphigus.

Subjects with pemphigus including subjects with severe and refractory pemphigus and/or with no response to current therapies are treated. Subjects positive for DSG1 and/or DSG3 may be treated. Further, subjects with severe and/or relapsed pernphigus treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of pemphigus, quantification of DSG1 and DSG3 antibodies, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of pemphigus may be assessed and recorded after treatment of selected subjects with anti-CD19 CAR T cell therapy.

### Example 9 Administration of Anti-CD19 CAR-Expressing Cells in Membranous Nephropathy (MN)

Therapeutic CAR+ T cell compositions containing autologous T cells expressing a chimeric antigen-receptor (CAR) specific for CD19 are administered to subjects with relapsing forms of membranous nephropathy (NIN),

Subjects with MN including subjects with severe and refractory MN and/or with no response to current therapies are treated. Subjects positive for PLA2R antibodies may be treated. Further, subjects with severe and/or relapsed MN treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of MN, quantification of PLA2R antibodies, assessment of proteinuria and eGFR, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of MN may be assessed and recorded after treatment of selected subjects with anti-CD19 CAR T cell therapy.

### Example 10 Administration of Anti-CD19 CAR-Expressing Cells in IgG4-related disease (12G4-RD)

Therapeutic CAR+ T cell compositions containing autologous T cells expressing a chimeric antigen-receptor (CAR) specific for CD19 are administered to subjects with relapsing forms of immunoglobulin G4-related disease (IgG4-RD).

Subjects with JgG4-RD including subjects with severe and refractory IgG4-RD and/or with no response to current therapies are treated. Subjects with elevated serum IgG4 levels may be treated. Further, subjects with severe and/or relapsed IgG4-RD treated with one or more prior lines of treatment are also treated. In some embodiment, the prior therapy is an anti-CD20 antibody, such as rituximab. In some embodiments, the subject to be treated is refractory to treatment with an anti-CD20 antibody, such as is a rituximab-refractory patient.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of IgG4-R=D, quantification of IgG4 antibodies, changes in the IgG4-RD responder index, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of IgG4-R D may be assessed and recorded after treatment of selected subjects with anti-CD19 CAR T cell therapy.

### Example 11 Administration of Anti-CD19 CAR-Expressing Cells in Neuromvelitis Optica spectrum disorder (NMOSD)

Therapeutic CAR+ T cell compositions containing autologous T cells expressing a chimeric antigen-receptor (CAR) specific for CD19 are administered to subjects with relapsing forms of neuromyelitis optica spectrum disorder (NMOSD).

Subjects with NMOSD including subjects with severe and refractory NMOSD and/or with no response to current therapies are treated. Subjects with elevated AQP4 antibody levels may be treated. Further, subjects with severe and/or relapsed NMOSD treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of NMOSD, quantification of AQP4 antibodies, changes in the EDSS scores, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of NMOSD may be assessed and recorded after treatment of selected subjects with anti-CD19 CAR T cell therapy.

### Example 12 Administration of Anti-CD19 CAR-Expressing Cells in Stiff-Person Syndrome (SPS)

Therapeutic CAR+ T cell compositions containing autologous T cells expressing a chimeric antigen-receptor (CAR) specific for CD19 are administered to subjects with relapsing forms of stiff person syndrome (SPS).

Subjects with SPS including subjects with severe and refractory SPS and/or with no response to current therapies are treated. Subjects with elevated GAD antibody levels may be treated. Further, subjects with severe and/or relapsed SPS treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of SPS, quantification of GAD antibodies, changes in DSI scores, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of SPS may be assessed and recorded after treatment of selected subjects with anti-CD19 CAR T cell therapy.

### Example 13 Administration of Anti-CD19 CAR-Expressing Cells in Irritable Bowel Disease (IBD)

Therapeutic CAR+ T cell compositions containing autologous T cells expressing a chimeric antigen-receptor (CAR) specific for CD19 are administered to subjects with relapsing forms of stiff person syndrome (IBD).

Subjects with IBD including subjects with severe and refractory IBD and/or with no response to current therapies are treated. Further, subjects with severe and/or relapsed IBD treated with one or more prior lines of treatment are also treated. In some embodiments, the subject to be treated is refractory to or has relapsed from treatment with an anti-CD20 antibody therapy, such as is refractory to or has relapsed following treatment with rituximab.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of IBD and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of IBD may be assessed and recorded after treatment of selected subjects with anti-CD19 CAR T cell therapy.

### Example 14 Administration of Anti-CD19 CAR-Expressing Cells in Thrombotic Thrombocytopenia Purpura (TTP)

Therapeutic CAR+ T cell compositions containing autologous T cells expressing a chimeric antigen-receptor (CAR) specific for CD19 are administered to subjects with relapsing forms of thrombotic thrombocytopenia purpura (TTP).

Subjects with TTP including subjects with severe and refractory TTP and/or with no response to current therapies are treated. Subjects with low activity of ADAMTS13 antibody may be treated. Further, subjects with severe and/or relapsed TTP treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of TTP, quantification of ADAMTS13 antibody activity, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of TTP may be assessed and recorded after treatment of selected subjects with anti-CD19 CAR T cell therapy.

### Example 15 Administration of Anti-CD19 CAR-Expressing Cells in Autoimmune Hemolytic Anemia (AIHA)

Therapeutic CAR+ T cell compositions containing autologous T cells expressing a chimeric antigen-receptor (CAR) specific for CD19 are administered to subjects with relapsing forms of autoimmune hemolytic anemia (AIHA).

Subjects with AIHA including subjects with severe and refractory AIHA and/or with no response to current therapies are treated. Further, subjects with severe and/or relapsed AIHA treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of AIHA and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of AIHA may be assessed and recorded after treatment of selected subjects with anti-CD19 CAR T cell therapy.

### Example 16 Administration of Anti-CD19 CAR-Expressing Cells in Immune Thrombocytopenia (ITP)

Therapeutic CAR+ T cell compositions containing autologous T cells expressing a chimeric antigen-receptor (CAR) specific for CD19 are administered to subjects with relapsing forms of immune thrombocytopenia (ITP).

Subjects with ITP including subjects with severe and refractory ITP and/or with no response to current therapies are treated. Subjects with detectable platelet-targeted antibodies may be treated. Further, subjects with severe and/or relapsed ITP treated with one or more prior lines of treatment are also treated.

In some embodiments, the subject is one characterized to have chronic ITP, that is ITP persisting for greater than twelve months and characterized by low platelet counts (e.g., less than about 100×10⁹/L, and in severe cases less than about 30×10⁹/L). The subject is a subject who has relapsed following, or are refractory to, one or more prior therapies, including but not limited to corticosteroids, intravenous immunoglobulin (IVIG), anti-D immunoglobulin, splenectomy, rituximab, thrombopoietin receptor agonists (e.g., romiplostim, eltrombopag, avatrombopag), and/or other immunosuppressive agents.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of ITP, quantification of platelet-targeted antibodies, and other laboratory tests. In particular, indicators of cITP may be assessed and for indicators of improvement including an increase in platelet count (for example, to at least about 50×109/L, or at least about 100×10⁹/), a reduction in bleeding manifestations such as petechiae, purpura, epistaxis, gingival bleeding, or menorrhagia, and improvement in bleeding scores (e.g., WHO bleeding scale or ITP Bleeding Score).

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of ITP may be assessed and recorded after treatment of selected subjects with anti-CD19 CAR T cell therapy.

### Example 17 Administration of Anti-CD19 CAR-Expressing Cells in IgA Nephropathy

Therapeutic CAR+ T cell compositions containing autologous T cells expressing a chimeric antigen-receptor (CAR) specific for CD19 are administered to subjects with relapsing forms of Immunoglobulin A (IgA) nephropathy.

Subjects with IgA including subjects with severe and refractory IgA and/or with no response to current therapies are treated. Subjects with High-risk proteinuria (>lg/g) may be treated. Further, subjects with severe and/or relapsed IgA treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of IgA, quantification of proteinuria, assessment of ESKD, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of IgA may be assessed and recorded after treatment of selected subjects with anti-CD19 CAR T cell therapy.

### Example 18 Administration of Anti-CD19 CAR-Expressing Cells in Bullous Pemphigoid

Therapeutic CAR+ T cell compositions containing autologous T cells expressing a chimeric antigen-receptor (CAR) specific for CD19 are administered to subjects with relapsing forms of bullous pemphigoid (BP.

Subjects with BP including subjects with severe and refractory BP and/or with no response to current therapies are treated. Subjects with high BPDAI scores (≥57) may be treated. Further, subjects with severe and/or relapsed BP treated with one or more prior lines of treatment are also treated.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring disease remission assessing the activity and severity of BP, quantification of BP180 levels, assessment of BPDAI score, and other laboratory tests.

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of BP may be assessed and recorded after treatment of selected subjects with anti-CD19 CAR T cell therapy.

### Example 19 Administration of Anti-CD19 CAR-Expressing Cells in Ulcerative Colitis (UC)

Therapeutic CAR+ T cell compositions containing autologous T cells expressing a chimeric antigen-receptor (CAR) specific for CD19 are administered to subjects with relapsing forms of ulcerative colitis (UC), including severe UC.

Subjects with UC including subjects with severe and refractory UC and/or with no response to current therapies are treated. Subjects with a high Mayo score (e.g., 11-12) or elevated Ulcerative Colitis Endoscopic Index of Severity (UCEIS) may be treated. The subject may be further characterized by objective markers of inflammation including elevated fecal calprotectin and hypoalbuminemia. Further, subjects with severe and/or relapsed UC treated with one or more prior lines of treatment are also treated, including those who have relapsed following, or are refractory to, one or more prior therapies, including but not limited to 5-aminosalicylates, corticosteroids (including corticosteroid-refractory or corticosteroid-dependent disease), immunomodulators (e.g., azathioprine or 6-mercaptopurine), biologics (e.g., anti-TNF agents, anti-integrin agents, or anti-IL-12/23 agents), and/or small molecule therapies such as Janus kinase (JAK) inhibitors.

Subjects are administered a single dose of the anti-CD19 CAR therapy described in Example 1 in a range from 0.5×10⁶ CAR-expressing viable T cells to 100×10⁶ CAR-expressing viable T cells. In particular, tested doses are in a range from 1×10⁶ CAR-expressing viable T cells to 50×10⁶ CAR-expressing viable T cells, such as 1×10⁶ CAR-expressing viable T cells, 5×10⁶ CAR-expressing viable T cells, 10×10⁶ CAR-expressing viable T cells, 25×10⁶ CAR-expressing viable T cells, and 50×10⁶ CAR-expressing viable T cells. Alternatively, subjects are administered a single dose of the anti-CD19 CAR therapy described in Example 1 in a range from 0.5×10⁶ CAR-expressing T cells to 100×10⁶ CAR-expressing T cells. In particular, tested doses are in a range from 1×10⁶ CAR-expressing T cells to 50×10⁶ CAR-expressing T cells, such as 1×10⁶ CAR-expressing T cells, 5×10⁶ CAR-expressing T cells, 10×10⁶ CAR-expressing T cells, 25×10⁶ CAR-expressing T cells, and 50×10⁶ CAR-expressing T cells. Prior to CAR+ T cell infusion, subjects may receive a lymphodepleting chemotherapy, such as fludarabine and cyclophosphamide for 3 days (e.g., flu, 30 mg/m²/day; and Cy, 300 mg/m²/day). The subjects typically receive CAR-expressing T cells 2-7 days after lymphodepletion.

Safety assessments are performed, including the assessment of treatment-emergent or serious adverse events (AEs), AEs of special interest, laboratory and/or imaging abnormalities, and Dose Limiting Toxicities (DLT).

Subjects are assessed for a reduction in symptoms and/or disease including by measuring a reduction in disease-associated symptoms such as stool frequency, rectal bleeding, abdominal pain, urgency, and nocturnal stools. Additional indicators of response may include objective evidence of mucosal healing, as measured endoscopically (e.g., reduced Mayo endoscopic subscore or Ulcerative Colitis Endoscopic Index of Severity) and/or histologically (e.g., reduced neutrophilic infiltration or resolution of crypt abscesses), and other laboratory tests such as for biomarkers of inflammation, such as decreased C-reactive protein, reduced fecal calprotectin, increased hemoglobin, and improved serum albumin..

Secondary endpoints related to efficacy and pharmacokinetics (PK) are also assessed, including, but not limited to, the list as follows: a number of subjects achieving no evidence of disease activity (NEDA), the maximum observed blood concentration or time of the maximum observed blood concentration, the area under the blood concentration-time curve from time zero to 28 days after dosing and/or the time to last measurable CAR-T concentrations (Tlast). Patient reported outcomes (PROs) are measured in the subject before and after administration of the CD19-targeted cell therapy. Change in status categories of improved, unchanged, worse, exacerbation, and died of UC may be assessed and recorded after treatment of selected subjects with anti-CD19 CAR T cell therapy.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO. | SEQUENCE | DESCRIPTION |
| 1 | RASQDISKYLN | CDR L1 |
| 2 | SRLHSGV | CDR L2 |
| 3 | GNTLPYTFG | CDR L3 |
| 4 | DYGVS | CDR H1 |
| 5 | VIWGSETTYYNSALKS | CDR H2 |
| 6 | YAMDYWG | CDR H3 |
| 7 | HYYYGGSYAMDY | CDR H3 |
| 8 | HTSRLHS | CDR L2 |
| 9 | QQGNTLPYT | CDR L3 |
| 10 | EVKLQESGPGLVAPSQSLSVTCTVSGVSLPDYGVSWIRQPPRKGL | VH |
| | EWLGVIWGSETTYYNSALKSRLTIIKDNSKSQVFLKMNSLQTDDT | |
| | AIYYCAKHYYYGGSYAMDYWGQGTSVTVSS | |
| 11 | DIQMTQTTSSLSASLGDRVTISCRASQDISKYLNWYQQKPDGTVK | VL |
| | LLIYHTSRLHSGVPSRFSGSGSGTDYSLTISNLEQEDIATYFCQQGN | |
| | TLPYTFGGGTKLEIT | |
| 12 | GSTSGSGKPGSGEGSTKG | Linker |
| 13 | gacatccagatgacccagaccacctccagcctgagcgccagcctgggcgaccgggtgaccatcagct | Sequence |
| | gccgggccagccaggacatcagcaagtacctgaactggtatcagcagaagcccgacggcaccgtcaa | encoding scFv |
| | gctgctgatctaccacaccagccggctgcacagcggcgtgcccagccggtttagcggcagcggctcc | |
| | ggcaccgactacagcctgaccatctccaacctggaacaggaagatatcgccacctacttttgccagcag | |
| | ggcaacacactgccctacacctttggcggcggaacaaagctggaaatcaccggcagcacctccggca | |
| | gcggcaagcctggcagcggcgagggcagcaccaagggcgaggtgaagctgcaggaaagcggccc | |
| | tggcctggtggcccccagccagagcctgagcgtgacctgcaccgtgagcggcgtgagcctgcccgac | |
| | tacggcgtgagctggatccggcagccccccaggaagggcctggaatggctgggcgtgatctggggca | |
| | gcgagaccacctactacaacagcgccctgaagagccggctgaccatcatcaaggacaacagcaagag | |
| | ccaggtgttcctgaagatgaacagcctgcagaccgacgacaccgccatctactactgcgccaagcacta | |
| | ctactacggcggcagctacgccatggactactggggccagggcaccagcgtgaccgtgagcagc | |
| 14 | DIQMTQTTSSLSASLGDRVTISCRASQDISKYLNWYQQKPDGTVK | scFv |
| | LLIYHTSRLHSGVPSRFSGSGSGTDYSLTISNLEQEDIATYFCQQGN | |
| | TLPYTFGGGTKLEITGSTSGSGKPGSGEGSTKGEVKLQESGPGLVA | |
| | PSQSLSVTCTVSGVSLPDYGVSWIRQPPRKGLEWLGVIWGSETTY | |
| | YNSALKSRLTIIKDNSKSQVFLKMNSLQTDDTAIYYCAKHYYYGG | |
| | SYAMDYWGQGTSVTVSS | |
| 15 | KASQNVGTNVA | CDR L1 |
| 16 | SATYRNS | CDR L2 |
| 17 | QQYNRYPYT | CDR L3 |
| 18 | SYWMN | CDR H1 |
| 19 | QIYPGDGDTNYNGKFKG | CDR H2 |
| 20 | KTISSVVDFYFDY | CDR H3 |
| 21 | EVKLQQSGAELVRPGSSVKISCKASGYAFSSYWMNWVKQRPGQ | VH |
| | GLEWIGQIYPGDGDTNYNGKFKGQATLTADKSSSTAYMQLSGLT | |
| | SEDSAVYFCARKTISSVVDFYFDYWGQGTTVTVSS | |
| 22 | DIELTQSPKFMSTSVGDRVSVTCKASQNVGTNVAWYQQKPGQSP | VL |
| | KPLIYSATYRNSGVPDRFTGSGSGTDFTLTITNVQSKDLADYFCQQ | |
| | YNRYPYTSGGGTKLEIKR | |
| 23 | GGGGSGGGGSGGGGS | Linker |
| 24 | EVKLQQSGAELVRPGSSVKISCKASGYAFSSYWMNWVKQRPGQ | scFv |
| | GLEWIGQIYPGDGDTNYNGKFKGQATLTADKSSSTAYMQLSGLT | |
| | SEDSAVYFCARKTISSVVDFYFDYWGQGTTVTVSSGGGGSGGGG | |
| | SGGGGSDIELTQSPKFMSTSVGDRVSVTCKASQNVGTNVAWYQQ | |
| | KPGQSPKPLIYSATYRNSGVPDRFTGSGSGTDFTLTITNVQSKDLA | |
| | DYFCQQYNRYPYTSGGGTKLEIKR | |
| 25 | TTTPAPRPPTPAPTIASQPLSLRPEACRPAAGGAVHTRGLDFACD | CD8a hinge |
| | | domain |
| 26 | IEVMYPPPYLDNEKSNGTIIHVKGKHLCPSPLFPGPSKP | CD28 hinge |
| | | domain |
| 27 | AAAIEVMYPPPYLDNEKSNGTIIHVKGKHLCPSPLFPGPSKP | CD28 hinge |
| | | domain |
| 28 | X₁PPX₂P | Hinge |
| | X₁ is glycinc, cystcinc or argininc | |
| | X₂ is cystcinc or thrconinc | |
| 29 | ESKYGPPCPPCP | spacer |
| | | (IgG4hinge) |
| | | (aa) |
| | | Homo sapiens |
| 30 | GAATCTAAGTACGGACCGCCCTGCCCCCCTTGCCCT | spacer |
| | | (IgG4hinge) |
| | | (nt) |
| | | homo sapiens |
| 31 | ESKYGPPCPPCPGQPREPQVYTLPPSQEEMTKNQVSLTCLVKGFY | Hinge-CH3 |
| | PSDIAVEWESNGQPENNYKTTPPVLDSDGSFFLYSRLTVDKSRWQ | spacer |
| | EGNVFSCSVMHEALHNHYTQKSLSLSLGK | Homo sapiens |
| 32 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVL | spacer |
| | HQDWLNGKEYKCKVSNKGLPSSIEKTISKAKGQPREPQVYTLPPS | Homo sapiens |
| | QEEMTKNQVSLTCLVKGFYPSDIAVEWESNGQPENNYKTTPPVL | |
| | DSDGSFFLYSRLTVDKSRWQEGNVFSCSVMHEALHNHYTQKSLS | |
| | LSLGK | |
| 33 | RWPESPKAQASSVPTAQPQAEGSLAKATTAPATTRNTGRGGEEK | IgD-hinge-Fc |
| | KKEKEKEEQEERETKTPECPSHTQPLGVYLLTPAVQDLWLRDKA | Homo sapiens |
| | TFTCFVVGSDLKDAHLTWEVAGKVPTGGVEEGLLERHSNGSQSQ | |
| | HSRLTLPRSLWNAGTSVTCTLNHPSLPPQRLMALREPAAQAPVKL | |
| | SLNLLASSDPPEAASWLLCEVSGFSPPNILLMWLEDQREVNTSGF | |
| | APARPPPQPGSTTFWAWSVLRVPAPPSPQPATYTCVVSHEDSRTL | |
| | LNASRSLEVSYVTDH | |
| 34 | FWVLVVVGGVLACYSLLVTVAFIIFWV | CD28 (amino |
| | | acids 153-179 |
| | | of Accession |
| | | No. P10747) |
| | | Homo sapiens |
| 35 | IEVMYPPPYLDNEKSNGTIIHVKGKHLCPSPLFPGPSKPFWVLVVV | CD28 (amino |
| | GGVLACYSLLVTVAFIIFWV | acids 114-179 |
| | | of Accession |
| | | No. P10747) |
| | | Homo sapiens |
| 36 | IYIWAPLAGTCGVLLLSLVITLYC | CD8α |
| | | transmembrane |
| | | domain |
| 37 | RVKFSRSADAPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 38 | RVKFSRSAEPPAYQQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 39 | RVKFSRSADAPAYKQGQNQLYNELNLGRREEYDVLDKRRGRDPE | CD3 zeta |
| | MGGKPRRKNPQEGLYNELQKDKMAEAYSEIGMKGERRRGKGHD | Homo sapiens |
| | GLYQGLSTATKDTYDALHMQALPPR | |
| 40 | RSKRSRLLHSDYMNMTPRRPGPTRKHYQPYAPPRDFAAYRS | CD28 (amino |
| | | acids 180-220 |
| | | of P10747) |
| | | Homo sapiens |
| 41 | RSKRSRGGHSDYMNMTPRRPGPTRKHYQPYAPPRDFAAYRS | CD28 (LL to GG) |
| | | Homo sapiens |
| 42 | KRGRKKLLYIFKQPFMRPVQTTQEEDGCSCRFPEEEEGGCEL | 4-1BB (amino |
| | | acids 214-255 |
| | | of Q07011.1) |
| | | Homo sapiens |
| 43 | MLLLVTSLLLCELPHPAFLLIPDIQMTQTTSSLSASLGDRVTISCRASQ | CD19 CAR |
| | DISKYLNWYQQKPDGTVKLLIYHTSRLHSGVPSRFSGSGSGTDYSLTIS | |
| | NLEQEDIATYFCQQGNTLPYTFGGGTKLEITGSTSGSGKPGSGEGSTKG | |
| | EVKLQESGPGLVAPSQSLSVTCTVSGVSLPDYGVSWIRQPPRKGLEWLG | |
| | VIWGSETTYYNSALKSRLTIIKDNSKSQVFLKMNSLQTDDTAIYYCAKH | |
| | YYYGGSYAMDYWGQGTSVTVSSESKYGPPCPPCPMFWVLVVVGGVLACY | |
| | SLLVTVAFIIFWVKRGRKKLLYIFKQPFMRPVQTTQEEDGCSCRFPEEE | |
| | EGGCELRVKESRSADAPAYQQGQNQLYNELNLGRREEYDVLDKRRGRDP | |
| | EMGGKPRRKNPQEGLYNELQKDKMAEAYSEIGMKGERRRGKGHDGLYQG | |
| | LSTATKDTYDALHMQALPPR | |
| 44 | atgctgctgctggtgaccagcctgctgctgtgcgagctgccccaccccgcctttctgctgatccccgaca | CD19 CAR |
| | tccagatgacccagaccacctccagcctgagcgccagcctgggcgaccgggtgaccatcagctgccg | |
| | ggccagccaggacatcagcaagtacctgaactggtatcagcagaagcccgacggcaccgtcaagctg | |
| | ctgatctaccacaccagccggctgcacagcggcgtgcccagccggtttagcggcagcggctccggca | |
| | ccgactacagcctgaccatctccaacctggaacaggaagatatcgccacctacttttgccagcagggca | |
| | acacactgccctacacctttggcggcggaacaaagctggaaatcaccggcagcacctccggcagcgg | |
| | caagcctggcagcggcgagggcagcaccaagggcgaggtgaagctgcaggaaagcggccctggc | |
| | ctggtggcccccagccagagcctgagcgtgacctgcaccgtgagcggcgtgagcctgcccgactacg | |
| | gcgtgagctggatccggcagccccccaggaagggcctggaatggctgggcgtgatctggggcagcg | |
| | agaccacctactacaacagcgccctgaagagccggctgaccatcatcaaggacaacagcaagagcca | |
| | ggtgttcctgaagatgaacagcctgcagaccgacgacaccgccatctactactgcgccaagcactacta | |
| | ctacggcggcagctacgccatggactactggggccagggcaccagcgtgaccgtgagcagcgaatct | |
| | aagtacggaccgccctgccccccttgccctatgttctgggtgctggtggtggtcggaggcgtgctggcct | |
| | gctacagcctgctggtcaccgtggccttcatcatcttttgggtgaaacggggcagaaagaaactcctgtat | |
| | atattcaaacaaccatttatgagaccagtacaaactactcaagaggaagatggctgtagctgccgatttcc | |
| | agaagaagaagaaggaggatgtgaactgcgggtgaagttcagcagaagcgccgacgcccctgccta | |
| | ccagcagggccagaatcagctgtacaacgagctgaacctgggcagaagggaagagtacgacgtcctg | |
| | gataagcggagaggccgggaccctgagatgggcggcaagcctcggcggaagaacccccaggaag | |
| | gcctgtataacgaactgcagaaagacaagatggccgaggcctacagcgagatcggcatgaagggcga | |
| | gcggaggcggggcaagggccacgacggcctgtatcagggcctgtccaccgccaccaaggataccta | |
| | cgacgccctgcacatgcaggccctgcccccaagg | |
| 45 | MALPVTALLLPLALLLHAARPDIQMTQTTSSLSASLGDRVTISCRA | CD19 CAR |
| | SQDISKYLNWYQQKPDGTVKLLIYHTSRLHSGVPSRFSGSGSGTD | |
| | YSLTISNLEQEDIATYFCQQGNTLPYTFGGGTKLEITGGGGSGGGG | |
| | SGGGGSEVKLQESGPGLVAPSQSLSVTCTVSGVSLPDYGVSWIRQ | |
| | PPRKGLEWLGVIWGSETTYYNSALKSRLTIIKDNSKSQVFLKMNS | |
| | LQTDDTAIYYCAKHYYYGGSYAMDYWGQGTSVTVSSTTTPAPRP | |
| | PTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSC | |
| | RFPEEEEGGCELRVKFSRSADAPAYKQGQNQLYNELNLGRREEY | |
| | DVLDKRRGRDPEMGGKPRRKNPQEGLYNELQKDKMAEAYSEIG | |
| | MKGERRRGKGHDGLYQGLSTATKDTYDALHMQALPPR | |
| 46 | MLLLVTSLLLCELPHPAFLLIPDIQMTQTTSSLSASLGDRVTISCRA | CD19 CAR |
| | SQDISKYLNWYQQKPDGTVKLLIYHTSRLHSGVPSRFSGSGSGTD | |
| | YSLTISNLEQEDIATYFCQQGNTLPYTFGGGTKLEITGSTSGSGKPG | |
| | SGEGSTKGEVKLQESGPGLVAPSQSLSVTCTVSGVSLPDYGVSWI | |
| | RQPPRKGLEWLGVIWGSETTYYNSALKSRLTIIKDNSKSQVFLKM | |
| | NSLQTDDTAIYYCAKHYYYGGSYAMDYWGQGTSVTVSSAAAIE | |
| | VMYPPPYLDNEKSNGTIIHVKGKHLCPSPLFPGPSKPFWVLVVVG | |
| | GVLACYSLLVTVAFIIFWVRSKRSRLLHSDYMNMTPRRPGPTRKH | |
| | YQPYAPPRDFAAYRSRVKFSRSADAPAYQQGQNQLYNELNLGRR | |
| | EEYDVLDKRRGRDPEMGGKPRRKNPQEGLYNELQKDKMAEAYS | |
| | EIGMKGERRRGKGHDGLYQGLSTATKDTYDALHMQALPPR | |
| 47 | FVPVFLPAKPTTTPAPRPPTPAPTIASQ | CD8a hinge and |
| | PLSLRPEACRPAAGGAVHTRGLDFACDI | transmembrane |
| | YIWAPLAGTCGVLLLSLVITLYCNHRN | |
| 48 | EIVLTQSPGTLSLSPGERATLSCRASQSVSSSYLAWYQQKPGQAPR | Anti-CD19 |
| | LLIYGASSRATGIPDRFSGSGSGTDFTLTISRLEPEDFAVYYCQQYG | variable light |
| | SSRFTFGPGTKVDIK | chain |
| 49 | GSTSGSGKPGSGEGSTKG | Linker |
| 50 | QVQLVQSGAEVKKPGSSVKVSCKDSGGTFSSYAISWVRQAPGQG | Anti-CD19 |
| | LEWMGGIIPIFGTTNYAQQFQGRVTITADESTSTAYMELSSLRSED | variable heavy |
| | TAVYYCAREAVAADWLDPWGQGTLVTVSS | chain |
| 51 | MALPVTALLLPLALLLHAARP | CD8a signal |
| | | sequence |
| 52 | MALPVTALLLPLALLLHAARPEIVLTQSPGTLSLSPGERATLSCRA | Anti-CD19 CAR |
| | SQSVSSSYLAWYQQKPGQAPRLLIYGASSRATGIPDRESGSGSGTD | |
| | FTLTISRLEPEDFAVYYCQQYGSSRFTFGPGTKVDIKGSTSGSGKP | |
| | GSGEGSTKGQVQLVQSGAEVKKPGSSVKVSCKDSGGTFSSYAIS | |
| | WVRQAPGQGLEWMGGIIPIFGTTNYAQQFQGRVTITADESTSTAY | |
| | MELSSLRSEDTAVYYCAREAVAADWLDPWGQGTLVTVSSFVPVF | |
| | LPAKPTTTPAPRPPTPAPTIASQPLSLRPEACRPAAGGAVHTRGLD | |
| | FACDIYIWAPLAGTCGVLLLSLVITLYCNHRNRSKRSRLLHSDYM | |
| | NMTPRRPGPTRKHYQPYAPPRDFAAYRSRVKFSRSADAPAYQQG | |
| | QNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 53 | MLLLVTSLLLCELPHPAFLLIPDIQMTQTTSSLSASLGDRVTISCRA | Anti-CD19 CAR |
| | SQDISKYLNWYQQ | |
| | KPDGTVKLLIYHTSRLHSGVPSRFSGSGSGTDYSLTISNLEQEDIAT | |
| | YFCQQGNTLPYTF | |
| | GGGTKLEITGSTSGSGKPGSGEGSTKGEVKLQESGPGLVAPSQSLS | |
| | VTCTVSGVSLPDYG | |
| | VSWIRQPPRKGLEWLGVIWGSETTYYNSALKSRLTIIKDNSKSQVF | |
| | LKMNSLQTDDTAIY | |
| | YCAKHYYYGGSYAMDYWGQGTSVTVSSAAAFVPVFLPAKPTTT | |
| | PAPRPPTPAPTIASQPL | |
| | SLRPEACRPAAGGAVHTRGLDFACDIYIWAPLAGTCGVLLLSLVI | |
| | TLYCNHRNRSKRSRL | |
| | LHSDYMNMTPRRPGPTRKHYQPYAPPRDFAAYRSRVKFSRSADA | |
| | PAYQQGQNQLYNELNL | |
| | GRREEYDVLDKRRGRDPEMGGKPRRKNPQEGLYNELQKDKMAE | |
| | AYSEIGMKGERRRGKGH | |
| | DGLYQGLSTATKDTYDALHMQALPPR | |
| 54 | MALPVTALLLPLALLLHAARPEIVLTQSPGTLSLSPGERATLSCRA | Anti-CD19 CAR |
| | SQSVSSSYLAWYQQ | |
| | KPGQAPRLLIYGASSRATGIPDRESGSGSGTDFTLTISRLEPEDFAV | |
| | YYCQQYGSSRFTF | |
| | GPGTKVDIKGSTSGSGKPGSGEGSTKGQVQLVQSGAEVKKPGSSV | |
| | KVSCKDSGGTFSSYA | |
| | ISWVRQAPGQGLEWMGGIIPIFGTTNYAQQFQGRVTITADESTSTA | |
| | YMELSSLRSEDTAV | |
| | YYCAREAVAADWLDPWGQGTLVTVSSFVPVFLPAKPTTTPAPRP | |
| | PTPAPTIASQPLSLRP | |
| | EACRPAAGGAVHTRGLDFACDIYIWAPLAGTCGVLLLSLVITLYC | |
| | NHRNRFSVVKRGRKK | |
| | LLYIFKQPFMRPVQTTQEEDGCSCRFPEEEEGGCELRVKFSRSADA | |
| | PAYQQGQNQLYNEL | |
| | NLGRREEYDVLDKRRGRDPEMGGKPRRKNPQEGLYNELQKDKM | |
| | AEAYSEIGMKGERRRGK | |
| | GHDGLYQGLSTATKDTYDALHMQALPPR | |
| 55 | QSVVTQPPSVSGAPGQRVTISCTGSSSNIGAGYDVHWYQQLPGTA | Anti-CD19 |
| | PKLLIYENTNRPSGV | variable light |
| | PDRFSGSKSGTSASLAITGLQAEDEADYYCQSYDSSLSGWRVFGG | chain |
| | GTKLTVLG | |
| 56 | MAEVQLVQSGAEVKKPGESLKISCKGSGYSFINSWIGWVRQMPG | Anti-CD19 |
| | KGLEWMGLIYPDDSDT | variable heavy |
| | RYSPSFQGQVTISADSAINTAYLQWSSLKASDTAMYYCARQSTYI | chain |
| | YGGYYDTWGQGTLVT | |
| | VSS | |
| 57 | QAVLTQPPSVSEAPRQRVTISCSGSSSNIGNNAVSWYQQLPGKAP | Anti-CD19 |
| | KLLIYYDDLLPSGVS | variable light |
| | DRFSGSKSGTSASLAISGLQSEDEADYYCAAWDDSLNGWVFGGG | chain |
| | TKVTVLG | |
| 58 | EVQLVQSGAEVKKPGESLKISCKGSGYSFTSYWIGWVRQMPGKG | Anti-CD19 |
| | LEWMGIIYPGDSDTRY | variable heavy |
| | SPSFQGQVTISADKSISTA YLQWSSLKASDTAMYYCARLSYSWSS | chain |
| | WYWDFWGQGTLVTVS | |
| | S | |
| 59 | QSVVTQPPSVSGAPGQRVTISCTGSSSNIGAGYDVHWYQQLPGTA | Anti-CD19 scFv |
| | PKLLIYENTNRPSGV | |
| | PDRFSGSKSGTSASLAITGLQAEDEADYYCQSYDSSLSGWRVFGG | |
| | GTKLTVLGSRGGGGS | |
| | GGGGSGGGGSLEMAEVQLVQSGAEVKKPGESLKISCKGSGYSFT | |
| | NSWIGWVRQMPGKGLE | |
| | WMGLIYPDDSDTRYSPSFQGQVTISADSAINTAYLQWSSLKASDT | |
| | AMYYCARQSTYIYGG | |
| | YYDTWGQGTLVTVSS | |
| 60 | QAVLTQPPSVSEAPRQRVTISCSGSSSNIGNNAVSWYQQLPGKAP | Anti-CD19 scFv |
| | KLLIYYDDLLPSGVS | |
| | DRFSGSKSGTSASLAISGLQSEDEADYYCAAWDDSLNGWVFGGG | |
| | TKVTVLGSRGGGGSGG | |
| | GGSGGGGSLEEVQLVQSGAEVKKPGESLKISCKGSGYSFTSYWIG | |
| | WVRQMPGKGLEWMGI | |
| | IYPGDSDTRYSPSFQGQVTISADKSISTAYLQWSSLKASDTAMYYC | |
| | ARLSYSWSSWYWDF | |
| | WGQGTLVTVSS | |
| 61 | MALPVTALLL PLALLLHAAR PEIVLTQSPG TLSLSPGERA | Anti-CD19 CAR |
| | TLSCRASQSVSSSYLAWYQQ KPGQAPRLLI YGASSRATGI | |
| | PDRFSGSGSG TDFTLTISRLEPEDFAVYYC QQYGSSRFTF | |
| | GPGTKVDIKG STSGSGKPGS GEGSTKGQVQLVQSGAEVKK | |
| | PGSSVKVSCK DSGGTFSSYA ISWVRQAPGQ GLEWMGGIIP | |
| | IFGTTNYAQQ FQGRVTITAD ESTSTAYMEL SSLRSEDTAV | |
| | YYCAREAVAADWLDPWGQGT LVTVSSFVPV FLPAKPTTTP | |
| | APRPPTPAPT IASQPLSLRPEACRPAAGGA VHTRGLDFAC | |
| | DIYIWAPLAG TCGVLLLSLV ITLYCNHRNRSKRSRLLHSD | |
| | YMNMTPRRPG PTRKHYQPYAPPRDFAAYRSRVKFSRSADA | |
| | PAYQQGQNQLYNELNLGRREEYDVLDKRRGRDPEMGGKPR | |
| | RKNPQEGLYNELQKDKMAEA YSEIGMKGER RRGKGHDGLY | |
| | QGLSTATKDT YDALHMQALPPR | |
| 62 | MALPVTALLL PLALLLHAAR PEIVLTQSPG TLSLSPGERA | Anti-CD19 CAR |
| | TLSCRASQSVSSSYLAWYQQ KPGQAPRLLI YGASSRATGI | |
| | PDRFSGSGSG TDFTLTISRLEPEDFAVYYC QQYGSSRFTF | |
| | GPGTKVDIKG STSGSGKPGS GEGSTKGQVQLVQSGAEVKK | |
| | PGSSVKVSCK DSGGTFSSYA ISWVRQAPGQ GLEWMGGIIP | |
| | IFGTTNYAQQ FQGRVTITAD ESTSTAYMEL SSLRSEDTAV | |
| | YYCAREAVAADWLDPWGQGT LVTVSSFVPV FLPAKPTTTP | |
| | APRPPTPAPT IASQPLSLRPEACRPAAGGA VHTRGLDFAC | |
| | DIYIWAPLAG TCGVLLLSLV ITLYCNHRNR | |
| | FSVVKRGRKK LLYIFKQPFM RPVQTTQEED GCSCRFPEEE | |
| | EGGCELRVKFSRSADAPAYQ QGQNQLYNELNLGRREEYDV | |
| | LDKRRGRDPE MGGKPRRKNPQEGLYNELQK | |
| | DKMAEAYSEIGMKGERRRGKGHDGLYQGLSTATKDTYDALHMQ | |
| | ALPPR | |
| 63 | MALPVTALLL PLALLLHAAR PEIVLTQSPG TLSLSPGERA | Anti-CD19 CAR |
| | TLSCRASQSVSSSYLAWYQQ KPGQAPRLLI YGASSRATGI | |
| | PDRFSGSGSG TDFTLTISRLEPEDFAVYYC QQYGSSRFTF | |
| | GPGTKVDIKG STSGSGKPGS GEGSTKGQVQLVQSGAEVKK | |
| | PGSSVKVSCK DSGGTFSSYA ISWVRQAPGQ GLEWMGGIIP | |
| | IFGTTNYAQQ FQGRVTITAD ESTSTAYMEL SSLRSEDTAV | |
| | YYCAREAVAADWLDPWGQGT LVTVSSFVPV FLPAKPTTTP | |
| | APRPPTPAPT IASQPLSLRPEACRPAAGGA VHTRGLDFAC | |
| | DIYIWAPLAG TCGVLLLSLV ITLYCNHRNQRRKYRSNKGE | |
| | SPVEPAEPCR YSCPREEEGS TIPIQEDYRK PEPACSPRVK | |
| | FSRSADAPAY QQGQNQLYNELNLGRREEYDVLDKRRGRDP | |
| | EMGGKPRRKNPQEGLYNELQ KDKMAEAYSE IGMKGERRRG | |
| | KGHDGLYQGL STATKDTYDALHMQALPPR | |
| 64 | MALPVTALLL PLALLLHAAR PEIVLTQSPG TLSLSPGERA | Anti-CD19 CAR |
| | TLSCRASQSVSSSYLAWYQQ KPGQAPRLLI YGASSRATGI | |
| | PDRFSGSGSG TDFTLTISRLEPEDFAVYYC QQYGSSRFTF | |
| | GPGTKVDIKG STSGSGKPGS GEGSTKGQVQLVQSGAEVKK | |
| | PGSSVKVSCK DSGGTFSSYA ISWVRQAPGQ | |
| | GLEWMGGIIPIFGTTNYAQQ | |
| | FQGRVTITADESTSTAYMELSSLRSEDTA VYYCAREAVAADWLD | |
| | PWGQGT LVTVSSFVPV FLPAKPTTTP APRPPTPAPT | |
| | IASQPLSLRPEACRPAAGGA VHTRGLDFAC DIYIWAPLAG | |
| | TCGVLLLSLV ITLYCNHRNRSKRSRLLHSD YMNMTPRRPG | |
| | PTRKHYQPYA PPRDFAAYRS QRRKYRSNKGESPVEPAEPC | |
| | RYSCPREEEG STIPIQEDYR KPEPACSPRV KESRSADAPA | |
| | YQQGQNQLYNELNLGRREEYDVLDKRRGRDPEMGGKPRRK | |
| | NPQEGLYNELQKDKMAEAYS EIGMKGERRRGKGHDGLYQG | |
| | LSTATKDTYD ALHMQALPPR | |
| 65 | MALPVTALLL PLALLLHAAR PEIVLTQSPG TLSLSPGERA | Anti-CD19 CAR |
| | TLSCRASQSV | |
| | SSSYLAWYQQ KPGQAPRLLI YGASSRATGI PDRFSGSGSG | |
| | TDFTLTISRL | |
| | EPEDFAVYYC QQYGSSRFTF GPGTKVDIKG STSGSGKPGS | |
| | GEGSTKGQVQ | |
| | LVQSGAEVKK PGSSVKVSCK DSGGTFSSYA ISWVRQAPGQ | |
| | GLEWMGGIIP | |
| | IFGTTNYAQQ FQGRVTITAD ESTSTAYMEL SSLRSEDTAV | |
| | YYCAREAVAA | |
| | DWLDPWGQGT LVTVSSFVPV FLPAKPTTTP APRPPTPAPT | |
| | IASQPLSLRP | |
| | EACRPAAGGA VHTRGLDFAC DIYIWAPLAG TCGVLLLSLV | |
| | ITLYCNHRNQ | |
| | RRKYRSNKGE SPVEPAEPCR YSCPREEEGS TIPIQEDYRK | |
| | PEPACSPRES | |
| | VVKRGRKKLL YIFKQPFMRP VQTTQEEDGC SCRFPEEEEG | |
| | GCELRVKFSR | |
| | SADAPAYQQG QNQLYNELNL GRREEYDVLD KRRGRDPEMG | |
| | GKPRRKNPQE | |
| | GLYNELQKDK MAEAYSEIGM KGERRRGKGH DGLYQGLSTA | |
| | TKDTYDALHM | |
| | QALPPR | |
| 66 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNRSKRSRL LHSDYMNMTP RRPGPTRKHY QPYAPPRDFA | |
| | AYRSRVKFSR | |
| | SADAPAYQQG QNQLYNELNL GRREEYDVLD KRRGRDPEMG | |
| | GKPRRKNPQE | |
| | GLYNELQKDK MAEAYSEIGM KGERRRGKGH DGLYQGLSTA | |
| | TKDTYDALHM | |
| | QALPPR | |
| 67 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNQRRKYRS NKGESPVEPA EPCRYSCPRE EEGSTIPIQE | |
| | DYRKPEPACS | |
| | PRFSVVKRGR KKLLYIFKQP FMRPVQTTQE EDGCSCRFPE | |
| | EEEGGCELRV | |
| | KFSRSADAPA YQQGQNQLYN ELNLGRREEY DVLDKRRGRD | |
| | PEMGGKPRRK | |
| | NPQEGLYNEL QKDKMAEAYS EIGMKGERRR GKGHDGLYQG | |
| | LSTATKDTYD | |
| | ALHMQALPPR | |
| 68 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNQRRKYRS NKGESPVEPA EPCRYSCPRE EEGSTIPIQE | |
| | DYRKPEPACS | |
| | PRVKFSRSAD APAYQQGQNQ LYNELNLGRR EEYDVLDKRR | |
| | GRDPEMGGKP | |
| | RRKNPQEGLY NELQKDKMAE AYSEIGMKGE RRRGKGHDGL | |
| | YQGLSTATKD | |
| | TYDALHMQAL PPR | |
| 69 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNRSKRSRL LHSDYMNMTP RRPGPTRKHY QPYAPPRDFA | |
| | AYRSQRRKYR | |
| | SNKGESPVEP AEPCRYSCPR EEEGSTIPIQ EDYRKPEPAC | |
| | SPRVKFSRSA | |
| | DAPAYQQGQN QLYNELNLGR REEYDVLDKR RGRDPEMGGK | |
| | PRRKNPQEGL | |
| | YNELQKDKMA EAYSEIGMKG ERRRGKGHDG LYQGLSTATK | |
| | DTYDALHMQA | |
| | LPPR | |
| 70 | MALPVTALLL PLALLLHAAR PEIVLTQSPG TLSLSPGERA | Anti-CD19 CAR |
| | TLSCRASQSV | |
| | SSSYLAWYQQ KPGQAPRLLI YGASSRATGI PDRESGSGSG | |
| | TDFTLTISRL | |
| | EPEDFAVYYC QQYGSSRFTF GPGTKVDIKG STSGSGKPGS | |
| | GEGSTKGQVQ | |
| | LVQSGAEVKK PGSSVKVSCK DSGGTFSSYA ISWVRQAPGQ | |
| | GLEWMGGIIP | |
| | IFGTTNYAQQ FQGRVTITAD ESTSTAYMEL SSLRSEDTAV | |
| | YYCAREAVAA | |
| | DWLDPWGQGT LVTVSSFVPV FLPAKPTTTP APRPPTPAPT | |
| | IASQPLSLRP | |
| | EACRPAAGGA VHTRGLDFAC DIYIWAPLAG TCGVLLLSLV | |
| | ITLYCNHRNR | |
| | SKRSRLLHSD YMNMTPRRPG PTRKHYQPYA PPRDFAAYRS | |
| | QRRKYRSNKG | |
| | ESPVEPAEPC RYSCPREEEG STIPIQEDYR KPEPACSPQV | |
| | RKAAITSYEK | |
| | SDGVYTGLST RNQETYETLK HEKPPQ | |
| 71 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNRSKRSRL LHSDYMNMTP RRPGPTRKHY QPYAPPRDFA | |
| | AYRSQRRKYR | |
| | SNKGESPVEP AEPCRYSCPR EEEGSTIPIQ EDYRKPEPAC | |
| | SPQVRKAAIT | |
| | SYEKSDGVYT GLSTRNQETY ETLKHEKPPQ | |
| 72 | MALPVTALLL PLALLLHAAR PEIVLTQSPG TLSLSPGERA | Anti-CD19 CAR |
| | TLSCRASQSV | |
| | SSSYLAWYQQ KPGQAPRLLI YGASSRATGI PDRFSGSGSG | |
| | TDFTLTISRL | |
| | EPEDFAVYYC QQYGSSRFTF GPGTKVDIKG STSGSGKPGS | |
| | GEGSTKGQVQ | |
| | LVQSGAEVKK PGSSVKVSCK DSGGTFSSYA ISWVRQAPGQ | |
| | GLEWMGGIIP | |
| | IFGTTNYAQQ FQGRVTITAD ESTSTAYMEL SSLRSEDTAV | |
| | YYCAREAVAA | |
| | DWLDPWGQGT LVTVSSFVPV FLPAKPTTTP APRPPTPAPT | |
| | IASQPLSLRP | |
| | EACRPAAGGA VHTRGLDFAC DIYIWAPLAG TCGVLLLSLV | |
| | ITLYCNHRNR | |
| | SKRSRLLHSD YMNMTPRRPG PTRKHYQPYA PPRDFAAYRS | |
| | QVRKAAITSY | |
| | EKSDGVYTGL STRNQETYET LKHEKPPQ | |
| 73 | MLLLVTSLLL CELPHPAFLL IPDIQMTQTT SSLSASLGDR | Anti-CD19 CAR |
| | VTISCRASQD | |
| | ISKYLNWYQQ KPDGTVKLLI YHTSRLHSGV PSRFSGSGSG | |
| | TDYSLTISNL | |
| | EQEDIATYFC QQGNTLPYTF GGGTKLEITG STSGSGKPGS | |
| | GEGSTKGEVK | |
| | LQESGPGLVA PSQSLSVTCT VSGVSLPDYG VSWIRQPPRK | |
| | GLEWLGVIWG | |
| | SETTYYNSAL KSRLTIIKDN SKSQVFLKMN SLQTDDTAIY | |
| | YCAKHYYYGG | |
| | SYAMDYWGQG TSVTVSSAAA FVPVFLPAKP TTTPAPRPPT | |
| | PAPTIASQPL | |
| | SLRPEACRPA AGGAVHTRGL DFACDIYIWA PLAGTCGVLL | |
| | LSLVITLYCN | |
| | HRNRSKRSRL LHSDYMNMTP RRPGPTRKHY QPYAPPRDFA | |
| | AYRSQVRKAA | |
| | ITSYEKSDGV YTGLSTRNQE TYETLKHEKP PQ | |
| 74 | EVQLQQSGAELVKPGASVKMSCKASGYTFTSYNMHWVKQTPGQ | Anti-CD20 Leu16 |
| | GLEWIGAIYPGNGDTSYNQKFKGKATLTADKSSSTAYMQLSSLTS | scFv heavy |
| | EDSADYYCARSNYYGSSYWFFDVWGAGTTVTVSS | chain |
| 75 | DIVLTQSPAILSASPGEKVTMTCRASSSVNYMDWYQKKPGSSPKP | Anti-CD20 Leu16 |
| | WIYATSNLASGVPARFSGSGSGTSYSLTISRVEAEDAATYYCQQW | scFv light |
| | SFNPPTFGGGTKLEIK | chain |
| | | variable region |
| 76 | EVQLVESGGG LVQPGRSLRL SCAASGFTFN DYAMHWVRQA | Anti-CD20 heavy |
| | PGKGLEWVST | chain |
| | ISWNSGSIGY ADSVKGRFTI SRDNAKKSLY LQMNSLRAED | |
| | TALYYCAKDI | |
| | QYGNYYYGMD VWGQGTTVTV SS | |
| 77 | EIVLTQSPAT LSLSPGERAT LSCRASQSVS SYLAWYQQKP | Anti-CD20 light |
| | GQAPRLLIYD | chain |
| | ASNRATGIPA RFSGSGSGTD FTLTISSLEP EDFAVYYCQQ | |
| | RSNWPITFGQ | |
| | GTRLEIK | |
| 78 | QVQLQQSGGG LVQPGGSLKL SCAASGIDFS RYWMSWVRRA | Anti-BCMA |
| | PGKGLEWIGE | heavy chain |
| | INPDSSTINY APSLKDKFII SRDNAKNTLY LQMSKVRSED | variable region |
| | TALYYCASLY | |
| | YDYGDAMDYW GQGTSVTVSS | |
| 79 | DIVMTQSQRF MTTSVGDRVS VTCKASQSVD SNVAWYQQKP | Anti-BCMA light |
| | RQSPKALIFS | chain variable |
| | ASLRFSGVPA RFTGSGSGTD FTLTISNLQS EDLAEYFCQQ | region |
| | YNNYPLTFGA | |
| | GTKLELK | |
| 80 | atgctccttctcgtgacctccctgcttctctgcgaactgccccatcctgccttcctgctg | Leader-CD19 VL- |
| | attcccgacattcagatgactcagaccacctcctccctgtccgcctccctgggcgaccgc | Whitlow linker |
| | gtgaccatctcatgccgcgccagccaggacatctcgaagtacctcaactggtaccagcag | CD19 VH |
| | aagcccgacggaaccgtgaagctcctgatctaccacacctcccggctgcacagcggagtg | (GGGGS)-5 CD20 |
| | ccgtctagattctcgggttcggggtcgggaactgactactcccttactatttccaacctg | VH (GGGGS)-3 |
| | gagcaggaggatattgccacctacttctgccaacaaggaaacaccctgccgtacactttt | CD20 VL |
| | ggcgggggaaccaagctggaaatcactggcagcacatccggttccgggaagcccggctcc | CD8 hinge+TM-4- |
| | ggagagggcagcaccaagggggaagtcaagctgcaggaatcaggacctggcctggtggcc | 1BB- CD3z |
| | ccgagccagtcactgtccgtgacttgtactgtgtccggagtgtcgctcccggattacgga | (Construct CAR |
| | gtgtcctggatcaggcagccacctcggaaaggattggaatggctcggagtcatctggggt | 1920) |
| | tccgaaaccacctattacaactcggcactgaaatccaggctcaccattatcaaggataac | |
| | tccaagtcacaagtgttcctgaagatgaatagcctgcagactgacgacacggcgatctac | |
| | tattgcgccaagcactactactacggcggatcctacgctatggactactggggccagggg | |
| | accagcgtgaccgtgtcatccggaggcggcggcagcggcgggggagggtccggagggggt | |
| | ggttctggtggaggaggatcgggaggcggtggcagcgaggtgcagttgcaacagtcagga | |
| | gctgaactggtcaagccaggagccagcgtgaagatgagctgcaaggcctccggttacacc | |
| | ttcacctcctacaacatgcactgggtgaaacagaccccgggacaagggctcgaatggatt | |
| | ggcgccatctaccccgggaatggcgatacttcgtacaaccagaagttcaagggaaaggcc | |
| | accctgaccgccgacaagagctcctccaccgcgtatatgcagttgagctccctgacctcc | |
| | gaggactccgccgactactactgcgcacggtccaactactatggaagctcgtactggttc | |
| | ttcgatgtctggggggccggcaccactgtgaccgtcagctccgggggcggaggatccggt | |
| | ggaggcggaagcgggggtggaggatccgacattgtgctgactcagtccccggcaatcctg | |
| | tcggcctcaccgggcgaaaaggtcacgatgacttgtagagcgtcgtccagcgtgaactac | |
| | atggattggtaccaaaagaagcctggatcgtcacccaagccttggatctacgctacatct | |
| | aacctggcctccggcgtgccagcgcggttcagcgggtccggctcgggcacctcatactcg | |
| | ctgaccatctcccgcgtggaggctgaggacgccgcgacctactactgccagcagtggtcc | |
| | ttcaacccgccgacttttggaggcggtactaagctggagatcaaagcggccgcaactacc | |
| | acccctgcccctcggccgccgactccggccccaaccatcgcaagccaacccctctccttg | |
| | cgccccgaagcttgccgcccggccgcgggtggagccgtgcatacccgggggctggacttt | |
| | gcctgcgatatctacatttgggccccgctggccggcacttgcggcgtgctcctgctgtcg | |
| | ctggtcatcaccctttactgcaagaggggccggaagaagctgctttacatcttcaagcag | |
| | ccgttcatgcggcccgtgcagacgactcaggaagaggacggatgctcgtgcagattccct | |
| | gaggaggaagaggggggatgcgaactgcgcgtcaagttctcacggtccgccgacgccccc | |
| | gcatatcaacagggccagaatcagctctacaacgagctgaacctgggaaggagagaggag | |
| | tacgacgtgctggacaagcgacgcggacgcgacccggagatgggggggaaaccacggcgg | |
| | aaaaaccctcaggaaggactgtacaacgaactccagaaagacaagatggcggaagcctac | |
| | tcagaaatcgggatgaagggagagcggaggaggggaaagggtcacgacgggctgtaccag | |
| | ggactgagcaccgccactaaggatacctacgatgccttgcatatgcaagcactcccaccccgg | |
| 81 | MLLLVTSLLLCELPHPAFLLIPDIQMTQTTSSLSASLGDRVTISCRA | Leader-CD19 VL- |
| | SQDISKYLNWYQQ | Whitlow linker |
| | KPDGTVKLLIYHTSRLHSGVPSRFSGSGSGTDYSLTISNLEQEDIAT | CD19 VH |
| | YFCQQGNTLPYTF | (GGGGS)-5 CD20 |
| | GGGTKLEITGSTSGSGKPGSGEGSTKGEVKLQESGPGLVAPSQSLS | VH (GGGGS)-3 |
| | VTCTVSGVSLPDYG | CD20 VL |
| | VSWIRQPPRKGLEWLGVIWGSETTYYNSALKSRLTIIKDNSKSQVF | CD8 hinge+TM-4- |
| | LKMNSLQTDDTAIY | 1BB- CD3z |
| | YCAKHYYYGGSYAMDYWGQGTSVTVSSGGGGSGGGGSGGGGS | (Construct CAR |
| | GGGGSGGGGSEVQLQQSG | 1920) |
| | AELVKPGASVKMSCKASGYTFTSYNMHWVKQTPGQGLEWIGAI | |
| | YPGNGDTSYNQKFKGKA | |
| | TLTADKSSSTAYMQLSSLTSEDSADYYCARSNYYGSSYWFFDVW | |
| | GAGTTVTVSSGGGGSG | |
| | GGGSGGGGSDIVLTQSPAILSASPGEKVTMTCRASSSVNYMDWY | |
| | QKKPGSSPKPWIYATS | |
| | NLASGVPARFSGSGSGTSYSLTISRVEAEDAATYYCQQWSFNPPTF | |
| | GGGTKLEIKAAATT | |
| | TPAPRPPTPAPTIASQPLSLRPEACRPAAGGAVHTRGLDFACDIYI | |
| | WAPLAGTCGVLLLS | |
| | LVITLYCKRGRKKLLYIFKQPFMRPVQTTQEEDGCSCRFPEEEEGG | |
| | CELRVKFSRSADAP | |
| | AYQQGQNQLYNELNLGRREEYDVLDKRRGRDPEMGGKPRRKNP | |
| | QEGLYNELQKDKMAEAY | |
| | SEIGMKGERRRGKGHDGLYQGLSTATKDTYDALHMQALPPR | |
| 82 | atgctccttctcgtgacctccctgcttctctgcgaactgccccatcctgccttcctgctg | Leader-CD20 VH |
| | attcccgaggtgcagttgcaacagtcaggagctgaactggtcaagccaggagccagcgtg | (GGGGS)3- |
| | aagatgagctgcaaggcctccggttacaccttcacctcctacaacatgcactgggtgaaa | CD20 VL- |
| | cagaccccgggacaagggctcgaatggattggcgccatctaccccgggaatggcgatact | (GGGGS)5 -CD19 |
| | tcgtacaaccagaagttcaagggaaaggccaccctgaccgccgacaagagctcctccacc | VL-Whitlow |
| | gcgtatatgcagttgagctccctgacctccgaggactccgccgactactactgcgcacgg | linker-CD19 |
| | tccaactactatggaagctcgtactggttcttcgatgtctggggggccggcaccactgtg | VH CD8 |
| | accgtcagctccgggggcggaggatccggtggaggcggaagcgggggtggaggatccgac | hinge+TM-4-1BB- |
| | attgtgctgactcagtccccggcaatcctgtcggcctcaccgggcgaaaaggtcacgatg | CD3z (Construct |
| | acttgtagagcgtcgtccagcgtgaactacatggattggtaccaaaagaagcctggatcg | 2019) |
| | tcacccaagccttggatctacgctacatctaacctggcctccggcgtgccagcgcggttc | |
| | agcgggtccggctcgggcacctcatactcgctgaccatctcccgcgtggaggctgaggac | |
| | gccgcgacctactactgccagcagtggtccttcaacccgccgacttttggaggcggtact | |
| | aagctggagatcaaaggaggcggcggcagcggcgggggagggtccggagggggtggttct | |
| | ggtggaggaggatcgggaggcggtggcagcgacattcagatgactcagaccacctcctcc | |
| | ctgtccgcctccctgggcgaccgcgtgaccatctcatgccgcgccagccaggacatctcg | |
| | aagtacctcaactggtaccagcagaagcccgacggaaccgtgaagctcctgatctaccac | |
| | acctcccggctgcacagcggagtgccgtctagattctcgggttcggggtcgggaactgac | |
| | tactcccttactatttccaacctggagcaggaggatattgccacctacttctgccaacaa | |
| | ggaaacaccctgccgtacacttttggcgggggaaccaagctggaaatcactggcagcaca | |
| | tccggttccgggaagcccggctccggagagggcagcaccaagggggaagtcaagctgcag | |
| | gaatcaggacctggcctggtggccccgagccagtcactgtccgtgacttgtactgtgtcc | |
| | ggagtgtcgctcccggattacggagtgtcctggatcaggcagccacctcggaaaggattg | |
| | gaatggctcggagtcatctggggttccgaaaccacctattacaactcggcactgaaatcc | |
| | aggctcaccattatcaaggataactccaagtcacaagtgttcctgaagatgaatagcctg | |
| | cagactgacgacacggcgatctactattgcgccaagcactactactacggcggatcctac | |
| | gctatggactactggggccaggggaccagcgtgaccgtgtcatccgcggccgcaactacc | |
| | acccctgcccctcggccgccgactccggccccaaccatcgcaagccaacccctctccttg | |
| | cgccccgaagcttgccgcccggccgcgggtggagccgtgcatacccgggggctggacttt | |
| | gcctgcgatatctacatttgggccccgctggccggcacttgcggcgtgctcctgctgtcg | |
| | ctggtcatcaccctttactgcaagaggggccggaagaagctgctttacatcttcaagcag | |
| | ccgttcatgcggcccgtgcagacgactcaggaagaggacggatgctcgtgcagattccct | |
| | gaggaggaagaggggggatgcgaactgcgcgtcaagttctcacggtccgccgacgccccc | |
| | gcatatcaacagggccagaatcagctctacaacgagctgaacctgggaaggagagaggag | |
| | tacgacgtgctggacaagcgacgcggacgcgacccggagatgggggggaaaccacggcgg | |
| | aaaaaccctcaggaaggactgtacaacgaactccagaaagacaagatggcggaagcctac | |
| | tcagaaatcgggatgaagggagagcggaggaggggaaagggtcacgacgggctgtaccag | |
| | ggactgagcaccgccactaaggatacctacgatgccttgcatatgcaagcactcccaccc | |
| | cgg | |
| 83 | MLLLVTSLLLCELPHPAFLLIPEVQLQQSGAELVKPGASVKMSCK | Leader-CD20 VH |
| | ASGYTFTSYNMHWVK | (GGGGS)3- |
| | QTPGQGLEWIGAIYPGNGDTSYNQKFKGKATLTADKSSSTAYMQ | CD20 VL- |
| | LSSLTSEDSADYYCAR | (GGGGS)5-CD19 |
| | SNYYGSSYWFFDVWGAGTTVTVSSGGGGSGGGGSGGGGSDIVLT | VL-Whitlow |
| | QSPAILSASPGEKVTM | linker-CD19 |
| | TCRASSSVNYMDWYQKKPGSSPKPWIYATSNLASGVPARFSGSG | VH CD8 |
| | SGTSYSLTISRVEAED | hinge+TM-4-1BB- |
| | AATYYCQQWSFNPPTFGGGTKLEIKGGGGSGGGGSGGGGSGGGG | CD3z amino acid |
| | SGGGGSDIQMTQTTSS | sequence |
| | LSASLGDRVTISCRASQDISKYLNWYQQKPDGTVKLLIYHTSRLH | (Construct |
| | SGVPSRFSGSGSGTD | CAR 2019) |
| | YSLTISNLEQEDIATYFCQQGNTLPYTFGGGTKLEITGSTSGSGKPG | |
| | SGEGSTKGEVKLQ | |
| | ESGPGLVAPSQSLSVTCTVSGVSLPDYGVSWIRQPPRKGLEWLGV | |
| | IWGSETTYYNSALKS | |
| | RLTIIKDNSKSQVFLKMNSLQTDDTAIYYCAKHYYYGGSYAMDY | |
| | WGQGTSVTVSSAAATT | |
| | TPAPRPPTPAPTIASQPLSLRPEACRPAAGGAVHTRGLDFACDIYI | |
| | WAPLAGTCGVLLLS | |
| | LVITLYCKRGRKKLLYIFKQPFMRPVQTTQEEDGCSCRFPEEEEGG | |
| | CELRVKFSRSADAP | |
| | AYQQGQNQLYNELNLGRREEYDVLDKRRGRDPEMGGKPRRKNP | |
| | QEGLYNELQKDKMAEAY | |
| | SEIGMKGERRRGKGHDGLYQGLSTATKDTYDALHMQALPPRPRT | |
| 84 | METDTLLLWV LLLWVPGSTG DIVLTQSPAI LSASPGEKVT | Anti-CD19/CD20 |
| | MTCRASSSVN | CAR |
| | YMDWYQKKPG SSPKPWIYAT SNLASGVPAR FSGSGSGTSY | |
| | SLTISRVEAE | |
| | DAATYYCQQW SFNPPTFGGG TKLEIKGSTS GGGSGGGSGG | |
| | GGSSEVQLQQ | |
| | SGAELVKPGA SVKMSCKASG YTFTSYNMHW VKQTPGQGLE | |
| | WIGAIYPGNG | |
| | DTSYNQKFKG KATLTADKSS STAYMQLSSL TSEDSADYYC | |
| | ARSNYYGSSY | |
| | WFFDVWGAGT TVTVSSGGGG SEVKLQESGP GLVAPSQSLS | |
| | VTCTVSGVSL | |
| | PDYGVSWIRQ PPRKGLEWLG VIWGSETTYY NSALKSRLTI | |
| | IKDNSKSQVF | |
| | LKMNSLQTDD TAIYYCAKHY YYGGSYAMDY WGQGTSVTVS | |
| | SGSTSGSGKP | |
| | GSGEGSTKGD IQMTQTTSSL SASLGDRVTI SCRASQDISK | |
| | YLNWYQQKPD | |
| | GTVKLLIYHT SRLHSGVPSR FSGSGSGTDY SLTISNLEQE | |
| | DIATYFCQQG | |
| | NTLPYTFGGG TKLEITESKY GPPCPPCPMF WVLVVVGGVL | |
| | ACYSLLVTVA | |
| | FIIFWVKRGR KKLLYIFKQP FMRPVQTTQE EDGCSCRFPE | |
| | EEEGGCELRV | |
| | KFSRSADAPA YQQGQNQLYN ELNLGRREEY DVLDKRRGRD | |
| | PEMGGKPRRK | |
| | NPQEGLYNEL QKDKMAEAYS EIGMKGERRR GKGHDGLYQG | |
| | LSTATKDTYD | |
| | ALHMQALPPR | |
| 85 | MALPVTALLLPLALLLHAARPEIVLTQSPATLSLSPGERATLSCRA | Anti-CD19/CD20 |
| | SQSVSSYLAWYQQKPGQAPRLLIYDASNRATGIPARFSGSGSGTD | CAR |
| | FTLTISSLEPEDFAVYYCQQRSNWPITFGQGTRLEIKGSTSGGGSG | |
| | GGSGGGGSSEVQLVESGGGLVQPGRSLRLSCAASGFTFNDYAMH | |
| | WVRQAPGKGLEWVSTISWNSGSIGYADSVKGRFTISRDNAKKSL | |
| | YLQMNSLRAEDTALYYCAKDIQYGNYYYGMDVWGQGTTVTVS | |
| | SGGGGSEVKLQESGPGLVAPSQSLSVTCTVSGVSLPDYGVSWIRQ | |
| | PPRKGLEWLGVIWGSETTYYNSALKSRLTIIKDNSKSQVFLKMNS | |
| | LQTDDTAIYYCAKHYYYGGSYAMDYWGQGTSVTVSSGSTSGSG | |
| | KPGSGEGSTKGDIQMTQTTSSLSASLGDRVTISCRASQDISKYLNW | |
| | YQQKPDGTVKLLIYHTSRLHSGVPSRFSGSGSGTDYSLTISNLEQE | |
| | DIATYFCQQGNTLPYTFGGGTKLEITESKYGPPCPPCPMFWVLVV | |
| | VGGVLACYSLLVTVAFIIFWVKRGRKKLLYIFKQPFMRPVQTTQE | |
| | EDGCSCRFPEEEEGGCELRVKFSRSADAPAYQQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR | |
| 86 | MLLLVTSLLLCELPHPAFLLIP | GMCSFR alpha |
| | | chain signal |
| | | sequence |
| 87 | atgcttctcctggtgacaagccttctgctctgtgagttaccacacccagcattcctcctgatccca | GMCSFR alpha |
| | | chain signal |
| | | sequence |
| 88 | MALPVTALLLPLALLLHA | CD8 alpha signal |
| | | peptide |

1. A method of treating a subject, the method comprising administering a dose of CD19-directed genetically modified T cells to a subject, wherein the T cells of the dose are positive for expression of a chimeric antigen receptor (CAR) that binds CD19 and the dose is from 1×10⁶ to 50×10⁶ CAR-positive T cells, wherein the subject is a subject:
(i) having or suspected of having Myasthenia gravis (MG) and has severe disease that is refractory to three or more prior therapies for treating MG;
(ii) having or suspected of having primary Sjogren's Disease (SiD), wherein the subject is characterized with refractory disease that is refractory to at least one prior therapy for treating SjD and/or with extraglandular disease;
(iii) having or suspected of having ANCA-associated vasculitis (AAV);
(iv) having or suspected of having Rheumatoid Arthritis (RA), wherein the subject has severe disease that is refractory to three or more prior therapies for treating RA;
(v) having or suspected of having Autoimmune Encephalitis (AE);
(vi) having or suspected of having Pemphigus:
(vii) having or suspected of having Membranous Nephropathy (MN);
(viii) having or suspected of having Immunoglobulin G4-related disease (IgG4-RD);
(ix) having or suspected of having Neuromvelitis optica spectrum disorder (NMOSD);
(x) having or suspected of having Stiff-person syndrome (SPS);
(xi) having or suspected of having Irritable bowel disease (IBD);
(xii) having or suspected of having Thrombotic Thrombocytopenia Purpura (TTP);
(xiii) having or suspected of having Autoimmune hemolytic anemia (AIHA);
(xiv) having or suspected of having Immune thrombocytopenia (ITP);
(xv) having or suspected of having chronic Immune thrombocytopenia (cITP);
(xvi) having or suspected of having IgA nephropathy;
(xvii) having or suspected of having bullous pemphigoid (BP); or
(xviii) having or suspected of having ulcerative colitis (UC).

2. A method for reducing disease activity, the method comprising administering a dose of CD19-directed genetically modified T cells to a subject, wherein the T cells of the dose are positive for expression of a chimeric antigen receptor (CAR) that binds CD19 and the dose is from 1×10⁶ to 50×10⁶ CAR-positive T cells, and the subject is a subject:
(i) having or suspected of having Myasthenia gravis (MG), wherein the subject has severe disease that is refractory to three or more prior therapies for treating MG,
(ii) having or suspected of having primary Sjogren's Disease (SjD), wherein the subject is characterized with refractory disease that is refractory to at least one prior therapy for treating SjD and/or with extraglandular disease:
(iii) having or suspected of having ANCA-associated vasculitis (AAV);
(iv) having or suspected of having Rheumatoid Arthritis (RA), wherein the subject has severe disease that is refractory to three or more prior therapies for treating RA:
(v) having or suspected of having Autoimmune Encephalitis (AE):
(vi) having or suspected of having Pemphigus:
(vii) having or suspected of having Membranous Nephropathy (MN);
(viii) having or suspected of having Immunoglobulin G4-related disease (IgG4-RD):
(ix) having or suspected of having Neuromyelitis optica spectrum disorder (NMOSD);
(x) having or suspected of having Stiff-person syndrome (SPS);
(xi) having or suspected of having Irritable bowel disease (IBD):
(xii) having or suspected of having Thrombotic Thrombocytopenia Purpura (TTP),
(xiii) having or suspected of having Autoimmune hemolytic anemia (AIHA):
(xiv) having or suspected of having Immune thrombocytopenia (ITP);
(xv) having or suspected of having chronic Immune thrombocytopenia (cITP);
(xvi) having or suspected of having IgA nephropathy;
(xvii) having or suspected of having bullous pemphigoid (BP); or
(xviii) having or suspected of having ulcerative colitis (UC).

3-19. (canceled)

20. The method of claim 1, wherein the subject has relapsed following remission after treatment with a prior therapy.

21-24. (canceled)

25. A method for post-induction maintenance therapy for treating ANCA-associated vasculitis (AAV) or Autoimmune Encephalitis (AE),
the method comprising administering a dose of CD19-directed genetically modified T cells to a subject having received an induction therapy for treating ANCA-associated vasculitis (AAV) or AE, respectively, wherein the T cells of the dose are positive for expression of a chimeric antigen receptor (CAR) that binds CD19 and the dose is from 1×10⁶ to 50×10⁶ CAR-positive.

26-37. (canceled)

38. The method of claim 1, wherein the subject has severe disease that is refractory to one or more prior therapies.

39. The method of claim 38, wherein the one or more prior therapy is two or more prior therapies.

40.-75. (canceled)

76. The method of claim 1, wherein the subject has a relapsed or refractory disease to one or more prior therapies for treating the disease.

77. The method of claim 76, wherein the one or more prior therapies are two or more prior therapies for treating the disease.

78. The method of claim 76, wherein at least one of the one or more prior therapies is an anti-CD20 antibody.

79. The method of claim 76, wherein the subject has a severe disease.

80-85. (canceled)

86. The method of claim 1, wherein the T cells are autologous to the subject.

87. The method of claim 1, further comprising obtaining a leukapheresis sample from the subject for manufacturing the composition comprising engineered T cells.

88. The method of claim 1, wherein prior to the administration, the subject has been preconditioned with a lymphodepleting therapy.

89. The method of claim 1, wherein the method further comprises, immediately prior to the administration of the dose of CD19-directed genetically modified T cells, administering a lymphodepleting therapy to the subject, wherein the lymphodepleting therapy comprises the administration of fludarabine and/or cyclophosphamide.

90-114. (canceled)

115. The method of claim 1, wherein the dose of T cells comprises CD4⁺ T cells expressing the CAR and CD8⁺ T cells expressing the CAR.

116. The method of claim 1, wherein the dose of T cells comprises CD4m T cells expressing the CAR and CD8m T cells expressing the CAR at a ratio between about 1:5.

117. The method of claim 1, wherein at least or at least about 90% of the T cells are CD3⁺ cells.

118-120. (canceled)

121. The method of claim 1, wherein at least 80% of the T cells are viable T cells.

122. (canceled)

123. The method of claim 1, wherein the subject is human.


SEQUENCE LISTING (88 sequences)

SEQ ID NO: 1 - protein, 11 aa, synthetic construct
  REGION 1..11: note = CDR L1
RASQDISKYLN

SEQ ID NO: 2 - protein, 7 aa, synthetic construct
  REGION 1..7: note = CDR L2
SRLHSGV

SEQ ID NO: 3 - protein, 9 aa, synthetic construct
  REGION 1..9: note = CDR L3
GNTLPYTFG

SEQ ID NO: 4 - protein, 5 aa, synthetic construct
  REGION 1..5: note = CDR H1
DYGVS

SEQ ID NO: 5 - protein, 16 aa, synthetic construct
  REGION 1..16: note = CDR H2
VIWGSETTYYNSALKS

SEQ ID NO: 6 - protein, 7 aa, synthetic construct
  REGION 1..7: note = CDR H3
YAMDYWG

SEQ ID NO: 7 - protein, 12 aa, synthetic construct
  REGION 1..12: note = CDR H3
HYYYGGSYAMDY

SEQ ID NO: 8 - protein, 7 aa, synthetic construct
  REGION 1..7: note = CDR L2
HTSRLHS

SEQ ID NO: 9 - protein, 9 aa, synthetic construct
  REGION 1..9: note = CDR L3
QQGNTLPYT

SEQ ID NO: 10 - protein, 120 aa, synthetic construct
  REGION 1..120: note = VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 11 - protein, 107 aa, synthetic construct
  REGION 1..107: note = VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 12 - protein, 18 aa, synthetic construct
  REGION 1..18: note = Linker
GSTSGSGKPGSGEGSTKG

SEQ ID NO: 13 - DNA, 735 bp, synthetic construct
  misc_feature 1..735: note = Sequence encoding scFv
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 14 - protein, 245 aa, synthetic construct
  REGION 1..245: note = scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 15 - protein, 11 aa, synthetic construct
  REGION 1..11: note = CDR L1
KASQNVGTNVA

SEQ ID NO: 16 - protein, 5 aa, synthetic construct
  REGION 1..5: note = CDR L2
SAYNS

SEQ ID NO: 17 - protein, 9 aa, synthetic construct
  REGION 1..9: note = CDR L3
QQYNRYPYT

SEQ ID NO: 18 - protein, 5 aa, synthetic construct
  REGION 1..5: note = CDR H1
SYWMN

SEQ ID NO: 19 - protein, 17 aa, synthetic construct
  REGION 1..17: note = CDR H2
QIYPGDGDTNYNGKFKG

SEQ ID NO: 20 - protein, 13 aa, synthetic construct
  REGION 1..13: note = CDR H3
KTISSVVDFYFDY

SEQ ID NO: 21 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 22 - protein, 106 aa, synthetic construct
  REGION 1..106: note = VL
DIELTQSPKFMSTSVGDRVSVTCKASQNVGTNVAWYQQKPGQSPKPLIYSAYNSGVPDRFTGSGSGTDFTLTITNVQSKDLADYFCQQYNRYPYTSGGGTKLEIKR

SEQ ID NO: 23 - protein, 15 aa, synthetic construct
  REGION 1..15: note = Linker
GGGGSGGGGSGGGGS

SEQ ID NO: 24 - protein, 243 aa, synthetic construct
  REGION 1..243: note = scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSAYNSGVPDRFTGSGSGTDFTLTITNVQSKDLADYFCQQYNRYPYTSGGGTKLEIKR

SEQ ID NO: 25 - protein, 45 aa, synthetic construct
  REGION 1..45: note = CD8Alpha hinge domain
TTTPAPRPPTPAPTIASQPLSLRPEACRPAAGGAVHTRGLDFACD

SEQ ID NO: 26 - protein, 39 aa, synthetic construct
  REGION 1..39: note = CD28 hinge domain
IEVMYPPPYLDNEKSNGTIIHVKGKHLCPSPLFPGPSKP

SEQ ID NO: 27 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 28 - skipped

SEQ ID NO: 29 - protein, 12 aa, Homo sapiens
  REGION 1..12: note = spacer (IgG4hinge) (aa) Homo sapiens
ESKYGPPCPPCP

SEQ ID NO: 30 - DNA, 36 bp, Homo sapiens
  misc_feature 1..36: note = spacer (IgG4hinge) (nt) Homo sapiens
  source 1..36: mol_type = unassigned DNA
gaatctaagtacggaccgccctgccccccttgccct

SEQ ID NO: 31 - protein, 117 aa, Homo sapiens
  REGION 1..117: note = Hinge-CH3 spacer Homo sapiens
ESKYGPPCPPCPGQPREPQVYTLPPSQEEMTKNQVSLTCLVKGFYPSDIAVEWESNGQPENNYKTTPPVLDSDGSFFKRLTVDKSRWQEGNVFSCSVMHEALHNHYTQKSLSLSLGK

SEQ ID NO: 32 - protein, 225 aa, Homo sapiens
  REGION 1..225: note = Hinge-CH2-CH3 spacer Homo sapiens
ESKYGPPCPPCPAPEFLGGPSVFLFPPKPKDTLMISRTPEVTCVVVDVSQEDPEVQFNWYVDGVEVHNAKTKPREEQFNSYVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFKRLTVDKSRWQEGNVFSCSVMHEALHNHYTQKSLSLSLGK

SEQ ID NO: 33 - protein, 282 aa, Homo sapiens
  REGION 1..282: note = IgD-hinge-Fc Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 34 - protein, 25 aa, Homo sapiens
  REGION 1..25: note = CD28 (amino acids 153-179 of Accession No. P10747) Homo sapiens
FWVLVVVGGVLACLLVTVAFIIFWV

SEQ ID NO: 35 - protein, 64 aa, Homo sapiens
  REGION 1..64: note = CD28 (amino acids 114-179 of Accession No. P10747) Homo sapiens
IEVMYPPPYLDNEKSNGTIIHVKGKHLCPSPLFPGPSKPFWVLVVVGGVLACLLVTVAFIIFWV

SEQ ID NO: 36 - protein, 24 aa, Homo sapiens
  REGION 1..24: note = CD8Alpha transmembrane domain
IYIWAPLAGTCGVLLLSLVITLYC

SEQ ID NO: 37 - protein, 108 aa, Homo sapiens
  REGION 1..108: note = CD3 zeta Homo sapiens
RVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 38 - protein, 108 aa, Homo sapiens
  REGION 1..108: note = CD3 zeta Homo sapiens
RVKFSRSAEPPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 39 - protein, 108 aa, Homo sapiens
  REGION 1..108: note = CD3 zeta Homo sapiens
RVKFSRSADAPAYKQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 40 - protein, 41 aa, Homo sapiens
  REGION 1..41: note = CD28 (amino acids 180-220 of P10747) Homo sapiens
RSKRSRLLHSDYMNMTPRRPGPTRKHYQPYAPPRDFAAYRS

SEQ ID NO: 41 - protein, 41 aa, Homo sapiens
  REGION 1..41: note = CD28 (LL to GG) Homo sapiens
RSKRSRGGHSDYMNMTPRRPGPTRKHYQPYAPPRDFAAYRS

SEQ ID NO: 42 - protein, 42 aa, Homo sapiens
  REGION 1..42: note = 4-1BB (amino acids 214-255 of Q07011.1) Homo sapiens
KRGRKKLLYIFKQPFMRPVQTTQEEDGCSCRFPEEEEGGCEL

SEQ ID NO: 43 - protein, 455 aa, synthetic construct
  REGION 1..455: note = CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 44 - DNA, 1383 bp, synthetic construct
  misc_feature 1..1383: note = CD19 CAR
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 45 - protein, 482 aa, synthetic construct
  REGION 1..482: note = CD19 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 46 - protein, 483 aa, synthetic construct
  REGION 1..483: note = CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 47 - protein, 83 aa, synthetic construct
  REGION 1..83: note = CD8a hinge and transmembrane
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 48 - protein, 108 aa, synthetic construct
  REGION 1..108: note = Anti-CD19 variable light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 49 - protein, 18 aa, synthetic construct
  REGION 1..18: note = Linker
GSTSGSGKPGSGEGSTKG

SEQ ID NO: 50 - protein, 119 aa, synthetic construct
  REGION 1..119: note = Anti-CD19 variable heavy chain
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 51 - protein, 21 aa, synthetic construct
  REGION 1..21: note = CD8a signal sequence
MALPVTALLLPLALLLHAARP

SEQ ID NO: 52 - protein, 498 aa, synthetic construct
  REGION 1..498: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 53 - protein, 502 aa, synthetic construct
  REGION 1..502: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 54 - protein, 504 aa, synthetic construct
  REGION 1..504: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 55 - protein, 113 aa, synthetic construct
  REGION 1..113: note = Anti-CD19 variable light chain
QSVVTQPPSVSGAPGQRVTISCTGSSSNIGAGYDVHWYQQLPGTAPKLLIYENTNRPSGVPDRFSGSKSGTSASLAITGLQAEDEADYYCQSYDSSLSGWRVFGGGTKLTVLG

SEQ ID NO: 56 - protein, 123 aa, synthetic construct
  REGION 1..123: note = Anti-CD19 variable heavy chain
MAEVQLVQSGAEVKKPGESLKISCKGSGYSFTNSWIGWVRQMPGKGLEWMGLIYPDDSDTRYSPSFQGQVTISADSAINTAYLQWSSLKASDTAMYYCARQSTYIYGGYYDTWGQGTLVTVSS

SEQ ID NO: 57 - protein, 111 aa, synthetic construct
  REGION 1..111: note = Anti-CD19 variable light chain
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLG

SEQ ID NO: 58 - protein, 121 aa, synthetic construct
  REGION 1..121: note = Anti-CD19 variable heavy chain
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 59 - protein, 255 aa, synthetic construct
  REGION 1..255: note = Anti-CD19 scFv
QSVVTQPPSVSGAPGQRVTISCTGSSSNIGAGYDVHWYQQLPGTAPKLLIYENTNRPSGVPDRFSGSKSGTSASLAITGLQAEDEADYYCQSYDSSLSGWRVFGGGTKLTVLGSRGGGGSGGGGSGGGGSLEMAEVQLVQSGAEVKKPGESLKISCKGSGYSFTNSWIGWVRQMPGKGLEWMGLIYPDDSDTRYSPSFQGQVTISADSAINTAYLQWSSLKASDTAMYYCARQSTYIYGGYYDTWGQGTLVTVSS

SEQ ID NO: 60 - protein, 251 aa, synthetic construct
  REGION 1..251: note = Anti-CD19 scFv
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLGSRGGGGSGGGGSGGGGSLEEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 61 - protein, 498 aa, synthetic construct; identical to SEQ ID NO: 52
  REGION 1..498: note = Anti-CD19 CAR

SEQ ID NO: 62 - protein, 504 aa, synthetic construct; identical to SEQ ID NO: 54
  REGION 1..504: note = Anti-CD19 CAR

SEQ ID NO: 63 - protein, 505 aa, synthetic construct
  REGION 1..505: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 64 - protein, 546 aa, synthetic construct
  REGION 1..546: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 65 - protein, 552 aa, synthetic construct
  REGION 1..552: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 66 - protein, 502 aa, synthetic construct; identical to SEQ ID NO: 53
  REGION 1..502: note = Anti-CD19 CAR

SEQ ID NO: 67 - protein, 556 aa, synthetic construct
  REGION 1..556: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 68 - protein, 511 aa, synthetic construct
  REGION 1..511: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGNELQKDKMAEAYSEIGMKGERRRGKGHDGLYQGLSTATKDTYDALHMQALPPR

SEQ ID NO: 69 - protein, 552 aa, synthetic construct
  REGION 1..552: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGNELQKDKMAEAYSEIGMKGERRRGKGHDGLYQGLSTATKDTYDALHMQALPPR

SEQ ID NO: 70 - protein, 476 aa, synthetic construct
  REGION 1..476: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQRRKYRSNKGESPVEPAEPCRYSCPREEEGSTIPIQEDYRKPEPACSPQVRKAAITSYEKSDGVYTGLSTRNQETYETLKHEKPPQ

SEQ ID NO: 71 - protein, 480 aa, synthetic construct
  REGION 1..480: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQRRKYRSNKGESPVEPAEPCRYSCPREEEGSTIPIQEDYRKPEPACSPQVRKAAITSYEKSDGVYTGLSTRNQETYETLKHEKPPQ

SEQ ID NO: 72 - protein, 428 aa, synthetic construct
  REGION 1..428: note = Anti-CD19 CAR
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQVRKAAITSYEKSDGVYTGLSTRNQETYETLKHEKPPQ

SEQ ID NO: 73 - protein, 432 aa, synthetic construct
  REGION 1..432: note = Anti-CD19 CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSQVRKAAITSYEKSDGVYTGLSTRNQETYETLKHEKPPQ

SEQ ID NO: 74 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 75 - protein, 104 aa, synthetic construct
  REGION 1..104: note = Anti-CD20 Leu16 scFv light chain variable region
DIVLTQSPAILSDGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 76 - protein, 122 aa, synthetic construct
  REGION 1..122: note = Anti-CD20 heavy chain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 77 - protein, 105 aa, synthetic construct
  REGION 1..105: note = Anti-CD20 light chain
EIVLTQSPATLSLSPGERATLSCRASQSVSSYLAWYQQKPGQAPRLLIYDNRATGIPARFSGSGSGTDFTLTISSLEPEDFAVYYCQQRSNWPITFGQGTRLEIK

SEQ ID NO: 78 - protein, 120 aa, synthetic construct
  REGION 1..120: note = Anti-BCMA heavy chain variable region
QVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 79 - protein, 107 aa, synthetic construct
  REGION 1..107: note = Anti-BCMA light chain variable region
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELK

SEQ ID NO: 80 - DNA, 2283 bp, synthetic construct
  misc_feature 1..2283: note = Leader-CD19 VL-Whitlow linker CD19 VH (GGGGS)-5 CD20 VH (GGGGS)-3 CD20 VL CD8 hinge+TM-4-1BB- CD3z (Construct CAR 1920)
  source 1..2283: mol_type = other DNA
atgctccttctcgtgacctccctgcttctctgcgaactgccccatcctgccttcctgctgattcccgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccggaggcggcggcagcggcgggggagggtccggagggggtggttctggtggaggaggatcgggaggcggtggcagcgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 81 - protein, 755 aa, synthetic construct
  REGION 1..755: note = Leader-CD19 VL-Whitlow linker CD19 VH (GGGGS)-5 CD20 VH (GGGGS)-3 CD20 VL CD8 hinge+TM-4-1BB- CD3z (Construct CAR 1920)
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGGGGSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSDGEKVTMTCRASSSVNYMDWYQKKPGSSPKPWIYATSNLASGVPARFSGSGSGTSYSLTISRVEAEDAATYYCQQWSFNPPTFGGGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 82 - DNA, 2283 bp, synthetic construct
  misc_feature 1..2283: note = Leader-CD20 VH (GGGGS)3 - CD20 VL -(GGGGS)5 -CD19 VL-Whitlow linker- CD19 VH CD8 hinge+TM-4-1BB- CD3z (Construct 2019)
  source 1..2283: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccgg

SEQ ID NO: 83 - protein, 758 aa, synthetic construct
  REGION 1..758: note = Leader-CD20 VH (GGGGS)3 - CD20 VL -(GGGGS)5 -CD19 VL-Whitlow linker- CD19 VH CD8 hinge+TM-4-1BB- CD3z amino acid sequence (Construct CAR 2019)
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSDGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPRPRT

SEQ ID NO: 84 - protein, 700 aa, synthetic construct
  REGION 1..700: note = Anti- CD19/CD20 CAR
MDTLLLWVLLLWVPGSTGDIVLTQSPAILSDGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 85 - protein, 704 aa, synthetic construct
  REGION 1..704: note = Anti- CD19/CD20 CAR
MALPVTALLLPLALLLHAARPEIVLTQSPATLSLSPGERATLSCRASQSVSSYLAWYQQKPGQAPRLLIYDNRATGIPARFSGSGSGTDFTLTISSLEPEDFAVYYCQQRSNWPITFGQGTRLEIKGSTSGGGSGGGSGGGGSSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGNELQKDKMAEAYSEIGMKGERRRGKGHDGQGLSTATKDTYDALHMQALPPR

SEQ ID NO: 86 - protein, 22 aa, synthetic construct
  REGION 1..22: note = GMCSFR alpha chain signal sequence
MLLLVTSLLLCELPHPAFLLIP

SEQ ID NO: 87 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 88 - protein, 18 aa, synthetic construct
  REGION 1..18: note = CD8 alpha signal peptide
MALPVTALLLPLALLLHA